(19) United States
(12) Patent Application Publication
(10) Pub. No.: US 2026/0215070 A1
(43) Pub. Date: Jul. 23, 2026

(54) ORGANIC LUMINESCENT COMPOUND AND ORGANIC ELECTROLUMINESCENT DEVICE USING SAME

(71) Applicant: SOLUS ADVANCED MATERIALS CO., LTD., Iksan-si, Jeollabuk-do (KR)

(72) Inventors: Uigeon LEE, Seongnam-si (KR); Jaeyi SIM, Seongnam-si (KR); Hyosuk SON, Seongnam-si (KR); Sangeun SIM, Seongnam-si (KR)

(73) Assignee: SOLUS ADVANCED MATERIALS CO., LTD., Iksan-si, Jeollabuk-do (KR)

(21) Appl. No.: 19/141,891

(22) PCT Filed: Dec. 21, 2023
(86) PCT No.: PCT/KR2023/021306

§ 371 Date: Jun. 20, 2025

(30) Foreign Application Priority Data
Dec. 23, 2022 (KR) 10-2022-0183796

(51) Int. Cl. H10K 50/11; C07D 403/04; C09K 11/06; H10K 50/16; H10K 85/60

(52) U.S. Cl. CPC H10K 50/11; C07D 403/04; C09K 11/06; H10K 50/16; H10K 85/622; H10K 85/626; H10K 85/633; H10K 85/654; H10K 85/6572; C09K 2211/1059

(57) ABSTRACT

The present invention relates to a novel compound having excellent carrier transport ability and thermal stability, and an organic electroluminescent device comprising the compound in one or more organic layers, thereby having improved properties such as luminous efficiency, driving voltage, lifespan, etc.

### TECHNICAL FIELD

The present invention relates to a novel organic compound and an organic electroluminescent device using the same and, more specifically, to a novel compound with excellent electron transporting ability, light emitting ability and thermal stability, and an organic electroluminescent device exhibiting improved characteristics, such as luminous efficiency, driving voltages, and lifespan, by containing the novel compound in one or more organic layers.

### BACKGROUND ART

In an organic electroluminescent device, upon the application of voltage between two electrodes, holes from an anode and electrons from a cathode are injected into organic layers. The injected holes and electrons combine with each other to form excitons, and the excitons fall down to the ground state to emit light. Particularly, materials used for the organic layers may be classified into light emission materials, hole injection materials, hole transport materials, electron transport materials, electron injection materials, and the like according to the function thereof.

Materials for forming an emissive layer of the organic electroluminescent device may be classified into blue, green and red light emission materials according to the color of light emission. Additionally, yellow and orange light emission materials may be used as light emission materials for displaying better natural colors. Additionally, host/dopant-based light emission materials may be used as light emission materials to increase color purity and improve luminous efficiency through energy transfer.

Dopant materials may be classified into fluorescent dopants using organic materials and phosphorescent dopants using metal complex compounds containing heavy atoms, such as Ir and Pt. These phosphorescent materials can theoretically improve the luminous efficiency up to four times compared to fluorescent materials, so research has been conducted on phosphorescent host materials as well as phosphorescent dopants.

Until today, NPB, BCP, Alq₃, and the like have been widely known as materials for use in a hole injection layer, a hole transport layer, and an electron transport layer, and anthracene derivatives have been reported as the light emitting layer materials. In particular, among the light emitting layer materials, metal complex compounds containing Ir, such as Firpic, Ir(ppy)₃, (acac)Ir(btp)₂, and the like, which have advantages in terms of improving efficiency, are used as blue, green, and red phosphorescent dopant material, and 4,4-dicarbazollybiphenyl (CBP) is used as the phosphorescent host material.

However, conventional light emission materials have advantages in terms of light emission characteristics, but are not satisfactory in terms of lifespan of organic electroluminescent devices due to low glass transition temperatures and very poor thermal stability. Accordingly, there is a need to develop light emission materials with excellent performance.

### DISCLOSURE OF INVENTION

### Technical Problem

The present invention has been made to solve the above-mentioned problems, and an aspect of the present invention is to provide a novel compound, which can be used as a material for an organic layer of an organic electroluminescent device, specifically, a material for an electron transport layer, and or an auxiliary electron transport layer, due to excellent electron injection and transport ability, electrochemical stability, thermal stability and the like.

Another aspect of the present invention is to provide an organic electroluminescent device exhibiting a low driving voltage, high luminous efficiency, and improved lifespan by containing the above-described novel compound.

Other purposes and advantages of the present disclosure will be clarified by following detailed description and claims.

### Solution to Problem

To achieve the above objectives, the present invention provides a compound represented by the following Chemical Formula 1:

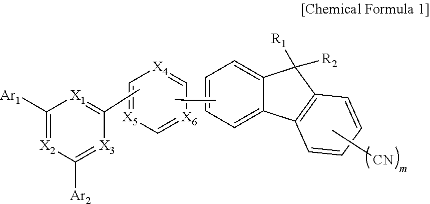

wherein in Chemical Formula 1,
X₁ to X₆ are the same as or different from each other and are each independently C(R₃) or N, provided that at least one of X₁ to X₃ and at least one of X₄ to X₆ are N;
Ar₁ and Ar₂ are the same as or different from each other and are each independently selected from the group consisting of a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ arylphosphine group, and a C₆-C₆₀ arylphosphine oxide group;
R₁ and R₂ are the same as or different from each other and are each independently a C₁-C₄₀ alkyl group, or an aliphatic hydrocarbon ring formed by bonding with any adjacent substituents;
R₃ is selected from the group consisting of a hydrogen, a deuterium (D), a halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ aryl phosphine group, a C₆-C₆₀ arylphosphine oxide group, and a C₆-C₆₀ arylamine group, or a plurality of R₃ are the same as or different from each other;
m is an integer of 1 or 2; and
the alkyl group, alkenyl group, alkynyl group, aryl group, heteroaryl group, aryloxy group, alkyloxy group, cycloalkyl group, heterocycloalkyl group, alkylsilyl group, arylsilyl group, alkylboron group, arylboron group, arylphosphine group, arylphosphine oxide group, and arylamine group of Ar₁ to Ar₂, and R₁ to R₃ may be each independently substituted with at least one substituent selected from the group consisting of a deuterium (D), a halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 ring atoms, a C₆ to C₆₀ aryl group, a heteroaryl group having 5 to 60 ring atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and wherein when the substituent is present in a plural number, they may be the same or different from each other.

In addition, the present invention also provides an organic electroluminescent device including: an anode, a cathode, and one or more organic layers disposed between the anode and the cathode, wherein at least one of the one or more organic layers includes the compound represented by Chemical Formula 1.

In such a case, the organic layer including the compound represented by Chemical Formula 1 may be selected from a light emitting layer, a light emitting auxiliary layer, a hole injection layer, a hole transport layer, an electron injection layer, a lifespan improvement layer, an electron transport layer, and an auxiliary electron transport layer. In such a case, the compound represented by Chemical Formula 1 may be included as a material for at least one of a phosphorescent host material of a light emitting layer, an electron transport layer and an auxiliary electron transport layer.

### Effects of the Invention

According to an embodiment of the present invention, a compound represented by Chemical Formula 1 has excellent characteristics such as an electron transport ability, luminescence ability, heat resistance, and the like, and thereby is applicable as an organic layer material of an organic electroluminescent device.

In particular, when the compound represented by Chemical Formula 1 of the present invention is used as an electron transport layer material or an auxiliary electron transport layer material, it may exhibit high thermal stability, low driving voltage, rapid mobility, high current efficiency and long lifespan characteristics compared to conventional host materials or electron transport materials.

Accordingly, the organic electroluminescent device including the compound of Chemical Formula 1 may be significantly improved in aspects such as excellent light emitting performance, low driving voltage, long lifespan, and high efficiency, and thus may be effectively applied to full color display panels and the like.

Effects according to the present invention are not limited by the description exemplified above, and more diverse effects are included in the present specification.

### DETAILED DESCRIPTION OF THE INVENTION

Hereinafter, the present invention will be described in detail.

### <Novel Organic Compound>

The present invention provides a novel compound capable of implementing low driving voltage, high luminescent efficiency, and long lifespan characteristics of a device through excellent electron transport capability and thermal stability.

Specifically, the compound contains an azine-based moiety advantageous in electron transporting capability and a fluorene-based moiety substituted with at least one cyano group, wherein these moieties are linked through another azine-based linker having an electron withdrawing group (EWG) to form a basic skeleton.

The compound of Chemical Formula 1 with such a structure contains at least two functional groups having strong electron withdrawing group (EWG), such as triazine or pyridine, as well as at least one cyano group, and thus has physicochemical properties that are more suitable for electron injection or electron transport. Therefore, the application of the compound of Chemical Formula 1 as a material for an electron transport layer or electron transport auxiliary layer enables the layers to favorably receive electrons from a cathode to smoothly transport the electrons to an emissive layer, and consequently, can lower the driving voltage of the device and induce high efficiency and long lifespan characteristics of the device.

Particularly, in the present invention, the introduction of, as a linker that links an azine-based moiety (e.g., triazine) and a fluorene-based moiety substituted with a cyano group, another azine-based linker (e.g., pyridine) having EWG characteristics can improve the electron flow compared with existing compounds having the same structure containing an arylene-based linker and achieve low LUMO due to high electron density, thereby providing an excellent electron transporting layer material with fast electron transporting capability. This compound, when applied to devices, can ensure low driving voltage and high efficiency characteristics of the devices.

Additionally, the present invention may adopt a structure in which an aliphatic hydrocarbon ring group is substituted at the 9-position of the fluorene moiety substituted with a cyano group. This aliphatic hydrocarbon ring group is chemically and physically stable, and thus can increase the stability of the molecular structure, thereby contributing to high efficiency and long lifespan characteristics. Accordingly, compared with an existing structure in which an alkyl or aryl group is substituted at the 9-position of the fluorene, the structure of the present invention can ensure structural and thermal stability and is therefore excellent in terms of glass transition temperature (Tg). Additionally, an aliphatic ring group, such as a cyclohexyl group, forms a uniform morphology due to its chair form, leading to excellent device characteristics.

Furthermore, the compound represented by Chemical Formula 1 has a high triplet energy and thus can prevent excitons generated from an emissive layer from diffusing (moving) into an electron transport layer or a hole transport layer adjacent to the emissive layer. Particularly, the introduction of strong EWG, such as cyanofluorene, triazine, or pyridine, into the compound may induce polarization within a material to form a wide bandgap, which can be utilized to block holes from the emissive layer, thereby enhancing the stability of the device.

Therefore, the number of excitons contributing to light emission in the emissive layer is increased to improve the luminous efficiency of the device and enhance the durability and stability of the device, thereby efficiently increasing the lifespan of the device. Most of the developed materials enable low-voltage driving, thereby exhibiting physical properties, such as increased lifespan.

As described above, the compound represented by Chemical Formula 1 of the present invention may be applicable as an organic layer material of an organic electroluminescent device, preferably a light-emitting layer material (a blue, a green and/or a red phosphorescent host material), an electron transport/injection layer material, a hole transport/injection layer material, a light-emitting auxiliary layer material, an auxiliary electron transport layer, and/or a lifespan enhancement layer material. In particular, when the compound of the present invention is used as a material for an electron transport layer or an electron transport auxiliary layer, excellent improvements in device efficiency, driving voltage, and lifespan characteristics can be expected. In addition, the organic electroluminescent device including the compound of the above chemical formula may be greatly improved in terms of the performance and lifespan characteristics, and the performance a full color organic electroluminescent panel to which the organic electroluminescent device is applied may also be maximized.

According to the present invention, the compound represented by Chemical Formula 1 comprises a nitrogen-containing heterocyclic ring (e.g., a X₁ to X₃-containing ring) and a fluorene-based moiety substituted with at least one cyano group as electron withdrawing groups (EWG) with excellent electron transport ability, and they are bonded through an azine-based linker having another EWG characteristics (e.g., X₄ to X₆-containing ring) to form a basic skeleton structure.

the nitrogen-containing heterocycle (e.g., X₁ to X₃-containing ring) may be a monocyclic heteroaryl group containing at least one nitrogen atoms. For an example of the nitrogen-containing heteroaromatic ring (e.g., X₁ to X₃-containing ring), X₁ to X₃ may be the same as or different from each other, and may each independently be C(R₃) or N, provided that at least one of X₁ to X₃ are N. For a specific example, X₁ to X₃ preferably includes two to three nitrogen atoms (N). As such, since the heterocycle containing two to three nitrogen atoms (N) is included, more excellent electron absorption characteristics may be exhibited, which is advantageous for electron injection and transport.

Herein, R₃ may be selected from the group consisting of a hydrogen, a deuterium (D), a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ monoarylphosphinyl group, a C₆ to C₆₀ diarylphosphinyl group, a C₆ to C₆₀ arylamine group, a C₅ to C₆₀ arylheteroarylamine group, and a heteroarylamine group having 5 to 60 nuclear atoms. In this case, when R₃ may be plural in number, the plurality of R₃ may be the same as or different from each other. Specifically, R₃ may preferably be selected from: a hydrogen, a deuterium (D), a C₁ to C₄₀ alkyl group, a C₆ to C₆₀ aryl group, and a heteroaryl group having 5 to 60 nuclear atoms.

In an embodiment of the present invention, the nitrogen-containing heterocycle may be more specifically embodied as any one of the following structural formulas. However, it is not limited thereto.

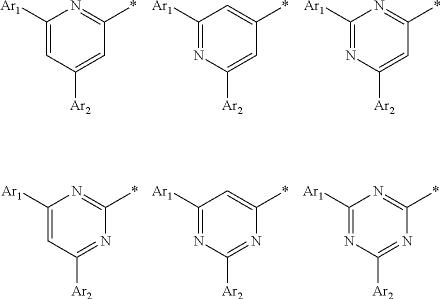

wherein the formulas,
* indicates a site where a bond with Chemical Formula 1 is made,
Ar₁ and Ar₂ are each as defined in Chemical Formula 1.
the nitrogen-containing heterocycle (e.g., X₁ to X₃-containing ring) according to the present invention may each be substituted with Ar₁ to Ar₂ as various substituents. Such Ar₁ and Ar₂ may be the same as or different from each other and may each independently be selected from: a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, or may be a moiety represented by the following Chemical Formula 2. specifically, Ar₁ and Ar₂ may be each independently selected from: a C₆ to C₁₈ aryl group, and a heteroaryl group having 5 to 18 nuclear atoms.

In an embodiment of the present invention, Ar₁ and Ar₂ may be the same as or different from each other and may each independently be more specifically embodied as any one of the following structural formulas. However, it is not limited thereto.

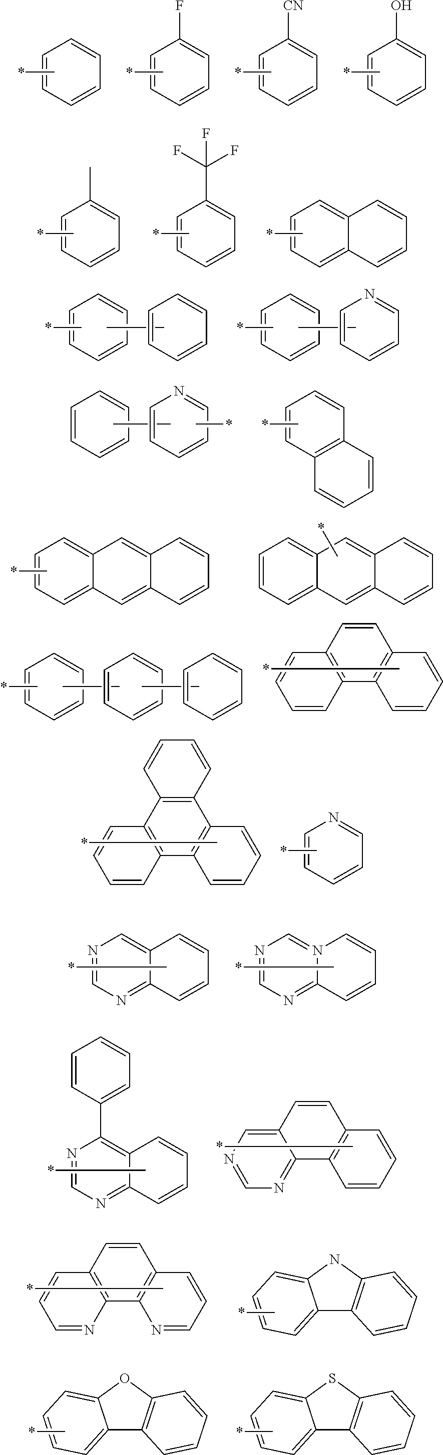

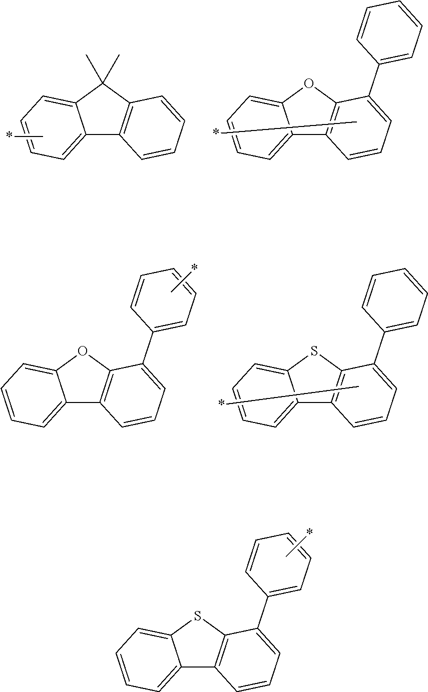

wherein the formulas,
* indicates a site where a bond with Chemical Formula 1 is made. In addition, although not shown in the above structural formulas, at least one substituent known in the art (e.g., the same as the definition of R₃) may be substituted.

According to the present invention, the compound represented by Chemical Formula 1 includes a fluorene-based moiety in which at least one cyano group is substituted on the other side of the nitrogen-containing heterocycle (e.g., X₁ to X₃-containing ring). The number (m) of cyano groups substituted in the fluorene-based moiety is not particularly limited, and for example, the number (m) of cyano groups substituted in the fluorene-based moiety may be 1 or 2.
the fluorene-based moiety according to the present invention may each be substituted with R₁ to R₂ as various substituents. Such R₁ to R₂ may be the same as or different from each other, and may each independently a C₁-C₄₀ alkyl group, or an aliphatic hydrocarbon ring formed by bonding with any adjacent substituents (e.g., R₁ to R₂).

As described above, the compound including the alkyl group or the aliphatic hydrocarbon ring at position 9 of the fluorene-based moiety may be represented by the following Chemical Formula 2 or Chemical Formula 3.

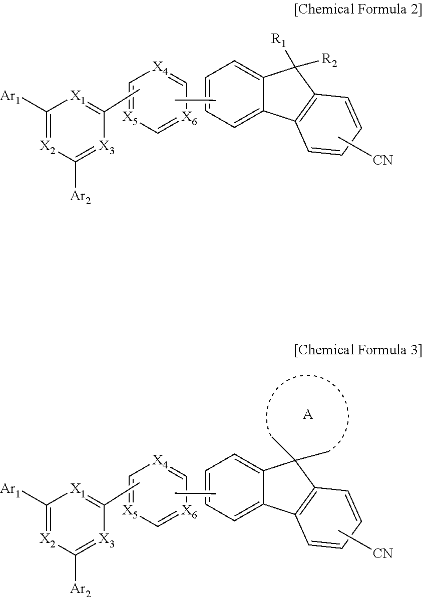

In Chemical Formula 2 or 3,
Ring A may be a conventional monocyclic or polycyclic aliphatic hydrocarbon-based ring known in the art, and specifically may be an aliphatic hydrocarbon ring group having 5 to 10 carbon atoms.

X₁ to X₆, Ar₁ to Ar₂, and R₁ to R₂ are each as defined in Chemical Formula 1.

In an embodiment of the present invention, the compound represented by Chemical Formula 3 may be more specifically embodied as any one of Chemical Formulas 3a to Chemical Formula 3d below. However, it is not limited thereto.

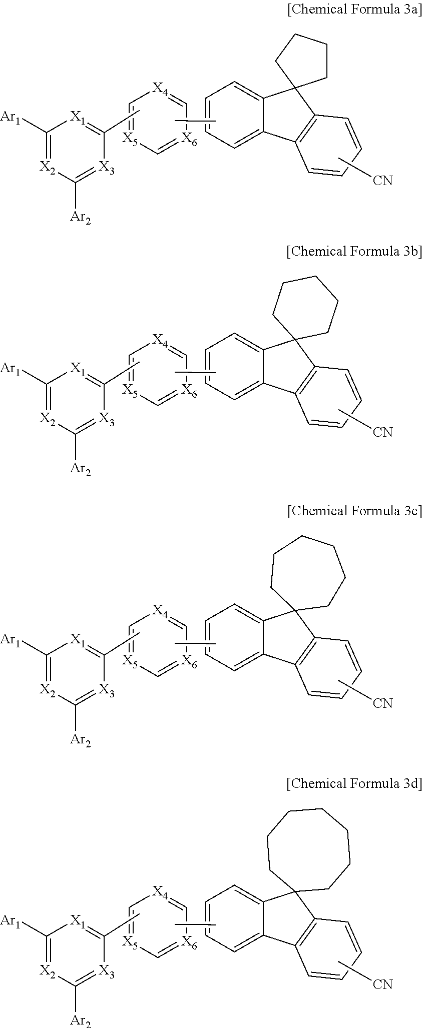

wherein the formulas 3a to 3d,
X₁ to X₆, and Ar₁ to Ar₂ are each as defined in Chemical Formula 1. In addition, although not shown in the above structural formulas, at least one substituent known in the art (e.g., the same as the definition of R₃) may be substituted.

In the compound represented by Chemical Formula 1 according to the present invention, a nitrogen-containing heterocyclic ring (e.g., a X₁ to X₃-containing ring) and a fluorene-based moiety substituted with at least one cyano group are linked through another azine-based linker (e.g., X₄ to X₆-containing ring). As such, when a separate linker (L) is present, a HOMO region may be expanded to give a benefit to a HOMO-LUMO distribution, and charge transfer efficiency may be increased through an appropriate overlap of HOMO-LUMO. Particularly, the azine-based linker (e.g., X₄ to X₆-containing ring) having EWG characteristics can improve the electron mobility compared with an existing arylene-based linker (e.g., phenylene or biphenylene) with EDG characteristics and implement low LUMO due to high electron density.

The azine-based linker (e.g., X₄ to X₆-containing ring) may be a monocyclic nitrogen-containing heteroarylene group containing at least one nitrogen atoms. For an example of the azine-based linker, X₄ to X₆ may be the same as or different from each other, and may each independently be C(R₃) or N, provided that at least two of X₄ to X₆ are N. For a specific example, X₄ to X₆ includes one to three nitrogen atoms (N), and preferably includes one nitrogen atom. Specific examples of the azine-based linker include a pyridine-based moiety, a pyrimidine-based moiety, a pyrazine-based moiety, a triazine-based moiety, and the like.

In an embodiment of the present invention, the azine-based linker (e.g., X₄ to X₆-containing ring moiety) may be more specifically embodied as any one of the following structural formulas. However, it is not limited thereto.

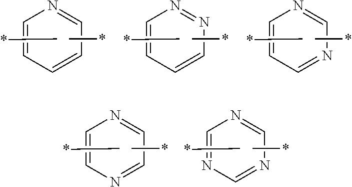

wherein the formulas,
* indicates a site where a bond with Chemical Formula 1 is made. In addition, although not shown in the above structural formulas, at least one substituent known in the art (e.g., the same as the definition of R₃) may be substituted.

In the above Chemical Formula 1, the alkyl, alkenyl, alkynyl, cycloalkyl, heterocycloalkyl, aryl, heteroaryl, alkyloxy, aryloxy, alkylsilyl, arylsilyl, alkylboron, arylboron, arylphosphine, arylphosphine oxide, and arylamine groups of Ar₁ to Ar₂, and R₁ to R₃ are optionally each independently unsubstituted or substituted with at least one selected from the group consisting of: a deuterium (D), a halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 nuclear atoms, a C₆-C₆₀ aryl group, a heteroaryl group having 5 to 60 nuclear atoms, a C₁-C₄₀ alkyloxy group, a C₆-C₆₀ aryloxy group, a C₁-C₄₀ alkylsilyl group, a C₆-C₆₀ arylsilyl group, a C₁-C₄₀ alkylboron group, a C₆-C₆₀ arylboron group, a C₁-C₄₀ arylphosphine group, a C₆-C₆₀ aryl phosphine oxide group, and a C₆-C₆₀ arylamine group, and provided that when the substituent is present in a plural number, they are optionally the same or different from each other.

In an embodiment of the present invention, the compound represented by Chemical Formula 1 may be more specifically represented by any one of the following Chemical Formulas 4 to 7 according to the bonding position of the cyano group substituted on the fluorene-based moiety. However, it is not limited thereto.

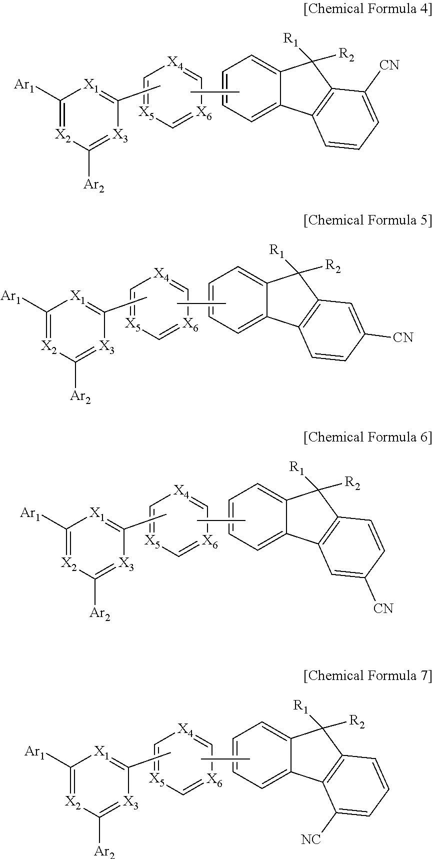

wherein the formulas 4 to 8,
X₁ to X₆, Ar₁ to Ar₂, and R₁ to R₂ are each as defined in Chemical Formula 1.

In another embodiment of the present invention, the compound represented by Chemical Formula 1 may be more specifically represented by any one of the following Chemical Formulas 8 to 11 according to the bonding position of the fluorene-based moiety connected to the azine-based linker (e.g., X₄ to X₆-containing ring). However, it is not limited thereto.

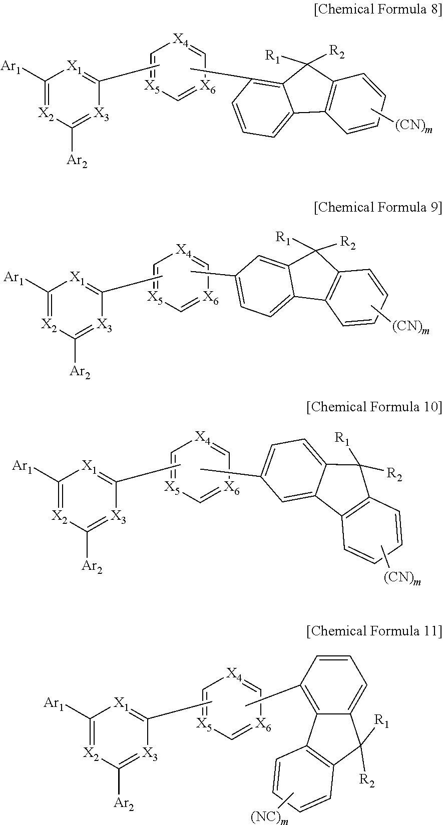

wherein the formulas 4 to 7,
X₁ to X₆, Ar₁ to Ar₂, R₁ to R₂ and m are each as defined in Chemical Formula 1.

In another embodiment of the present invention, the compound represented by Chemical Formula 1 may be more specifically represented by any one of the following Chemical Formulas 12 to 14 according to the type of the azine-based moiety (e.g., X₁ to X₃-containing ring) and the azine-based linker (e.g. X₄ to X₃-containing ring) and the bonding site thereof. However, it is not limited thereto.

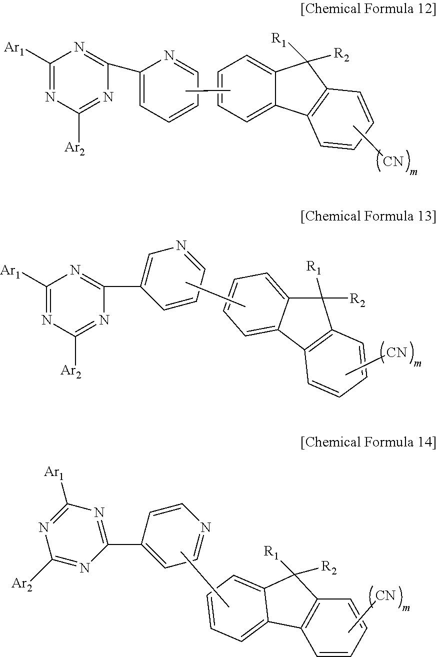

wherein the formulas 12 to 14,
Ar₁ to Ar₂, R₁ to R₂ and m are each as defined in Chemical Formula 1.

For example, the compound represented by Chemical Formulae 12 to 14 may be more specifically represented by any one of the following Chemical Formulae 12a to 14d.

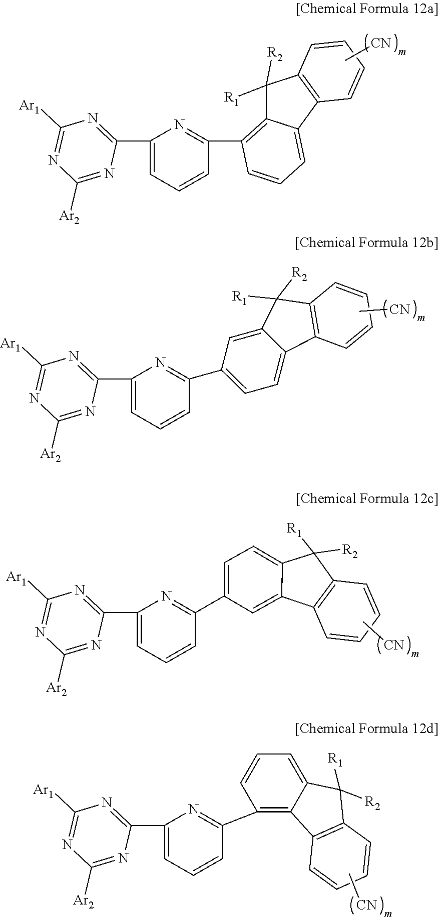

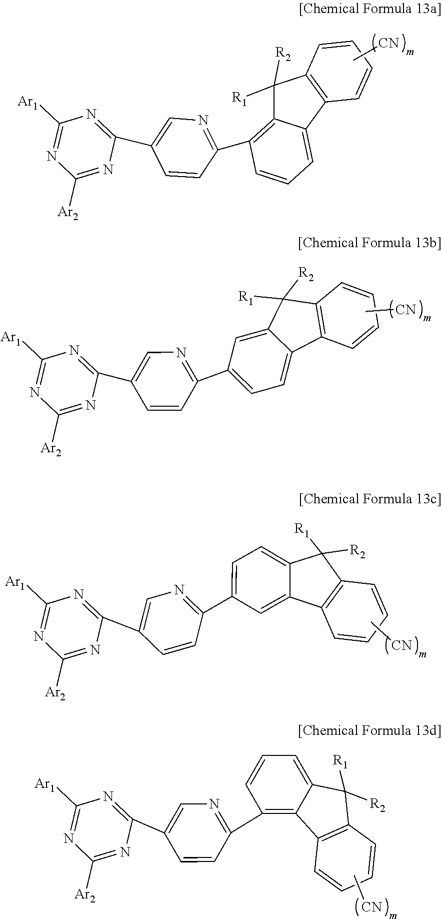

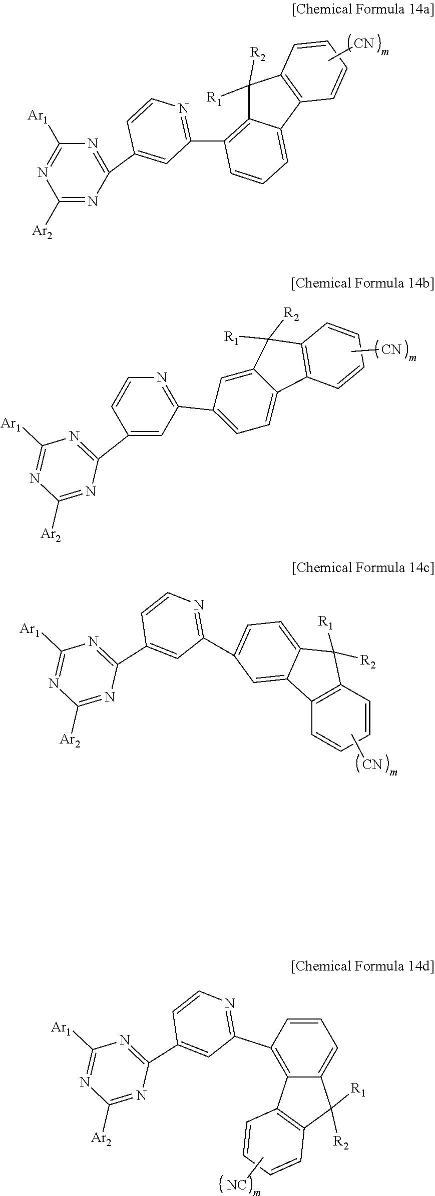

wherein the formulas 12a to 14d,
Ar₁ to Ar₂, R₁ to R₂ and m are each as defined in Chemical Formula 1.

The compound represented by Chemical Formula 1 of the present invention described above may be further embodied as compounds represented by the following compounds represented by, for example, 1 to 135. However, the compound represented by Chemical Formula 1 of the present invention is not limited to those exemplified below.

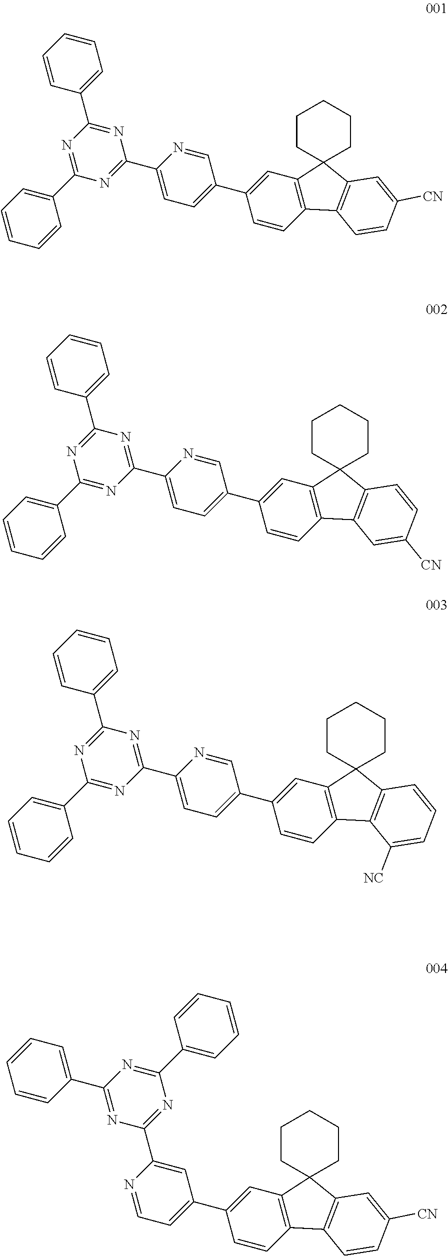 [SMILES: N#Cc1ccc2c(c1)C1(CCCCC1)c1cc(-c3ccc(-c4nc(-c5ccccc5)nc(-c5ccccc5)n4)nc3)ccc1-2 ; N#Cc1ccc2c(c1)-c1ccc(-c3ccc(-c4nc(-c5ccccc5)nc(-c5ccccc5)n4)nc3)cc1C21CCCCC1 ; N#Cc1cccc2c1-c1ccc(-c3ccc(-c4nc(-c5ccccc5)nc(-c5ccccc5)n4)nc3)cc1C21CCCCC1 ; N#Cc1ccc2c(c1)C1(CCCCC1)c1cc(-c3ccnc(-c4nc(-c5ccccc5)nc(-c5ccccc5)n4)c3)ccc1-2]

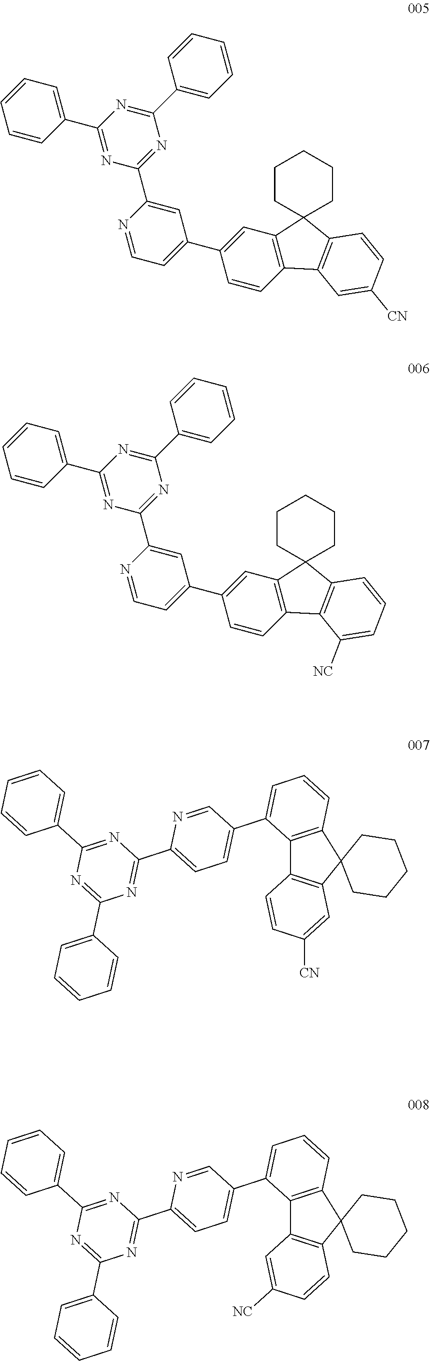 [SMILES: N#Cc1ccc2c(c1)-c1ccc(-c3ccnc(-c4nc(-c5ccccc5)nc(-c5ccccc5)n4)c3)cc1C21CCCCC1 ; N#Cc1cccc2c1-c1ccc(-c3ccnc(-c4nc(-c5ccccc5)nc(-c5ccccc5)n4)c3)cc1C21CCCCC1 ; N#Cc1ccc2c(c1)C1(CCCCC1)c1cccc(-c3ccc(-c4nc(-c5ccccc5)nc(-c5ccccc5)n4)nc3)c1-2 ; N#Cc1ccc2c(c1)-c1c(-c3ccc(-c4nc(-c5ccccc5)nc(-c5ccccc5)n4)nc3)cccc1C21CCCCC1]

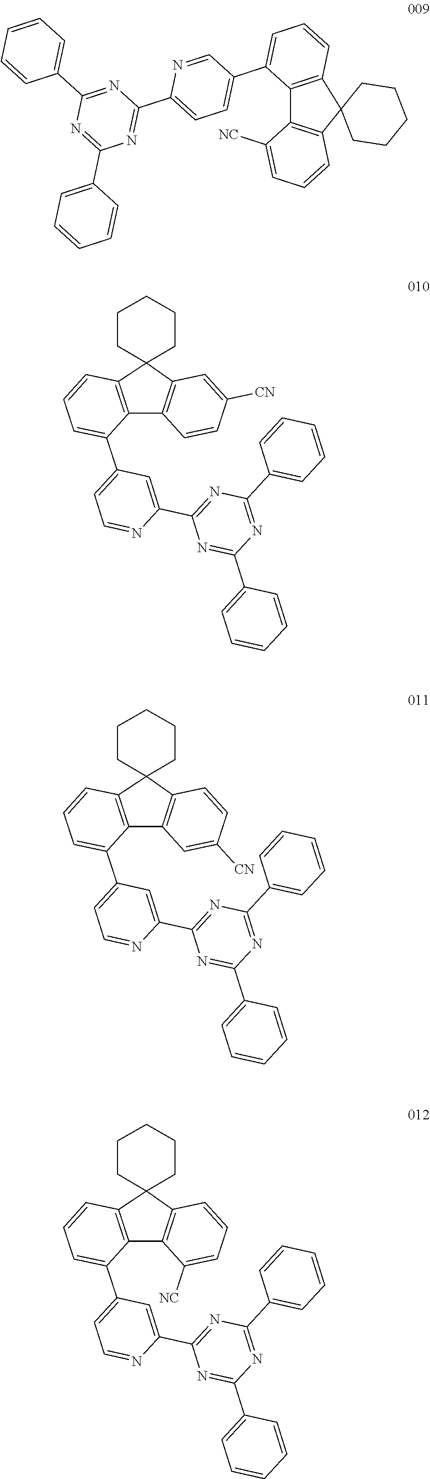 [SMILES: N#Cc1cccc2c1-c1c(-c3ccc(-c4nc(-c5ccccc5)nc(-c5ccccc5)n4)nc3)cccc1C21CCCCC1 ; N#Cc1ccc2c(c1)C1(CCCCC1)c1cccc(-c3ccnc(-c4nc(-c5ccccc5)nc(-c5ccccc5)n4)c3)c1-2 ; N#Cc1ccc2c(c1)-c1c(-c3ccnc(-c4nc(-c5ccccc5)nc(-c5ccccc5)n4)c3)cccc1C21CCCCC1 ; N#Cc1cccc2c1-c1c(-c3ccnc(-c4nc(-c5ccccc5)nc(-c5ccccc5)n4)c3)cccc1C21CCCCC1]

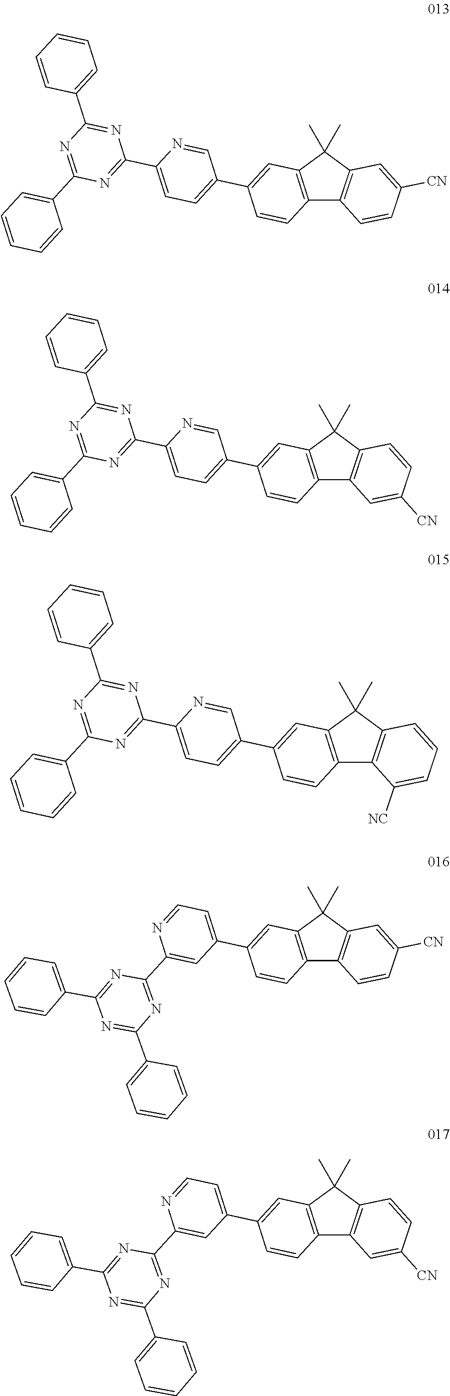 [SMILES: CC1(C)c2cc(C#N)ccc2-c2ccc(-c3ccc(-c4nc(-c5ccccc5)nc(-c5ccccc5)n4)nc3)cc21 ; CC1(C)c2ccc(C#N)cc2-c2ccc(-c3ccc(-c4nc(-c5ccccc5)nc(-c5ccccc5)n4)nc3)cc21 ; CC1(C)c2cc(-c3ccc(-c4nc(-c5ccccc5)nc(-c5ccccc5)n4)nc3)ccc2-c2c(C#N)cccc21 ; CC1(C)c2cc(C#N)ccc2-c2ccc(-c3ccnc(-c4nc(-c5ccccc5)nc(-c5ccccc5)n4)c3)cc21 ; CC1(C)c2ccc(C#N)cc2-c2ccc(-c3ccnc(-c4nc(-c5ccccc5)nc(-c5ccccc5)n4)c3)cc21]

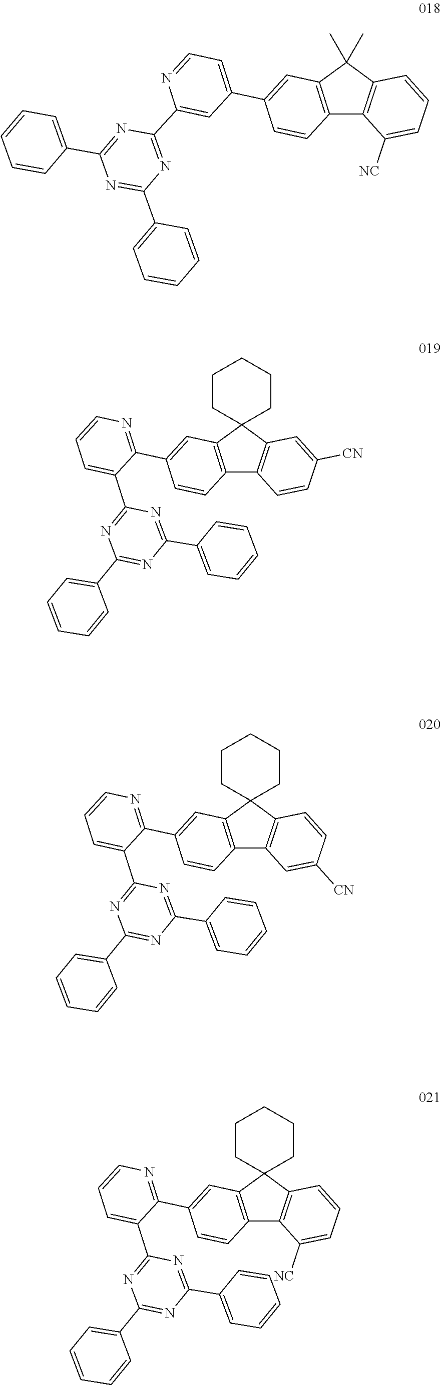 [SMILES: CC1(C)c2cc(-c3ccnc(-c4nc(-c5ccccc5)nc(-c5ccccc5)n4)c3)ccc2-c2c(C#N)cccc21 ; N#Cc1ccc2c(c1)C1(CCCCC1)c1cc(-c3ncccc3-c3nc(-c4ccccc4)nc(-c4ccccc4)n3)ccc1-2 ; N#Cc1ccc2c(c1)-c1ccc(-c3ncccc3-c3nc(-c4ccccc4)nc(-c4ccccc4)n3)cc1C21CCCCC1 ; N#Cc1cccc2c1-c1ccc(-c3ncccc3-c3nc(-c4ccccc4)nc(-c4ccccc4)n3)cc1C21CCCCC1]

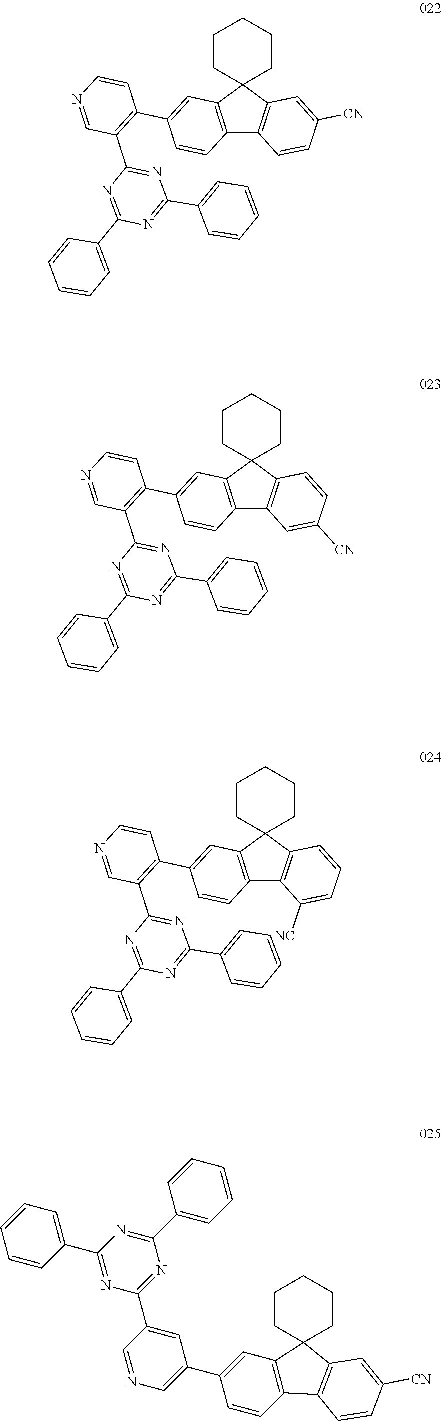 [SMILES: N#Cc1ccc2c(c1)C1(CCCCC1)c1cc(-c3ccncc3-c3nc(-c4ccccc4)nc(-c4ccccc4)n3)ccc1-2 ; N#Cc1ccc2c(c1)-c1ccc(-c3ccncc3-c3nc(-c4ccccc4)nc(-c4ccccc4)n3)cc1C21CCCCC1 ; N#Cc1cccc2c1-c1ccc(-c3ccncc3-c3nc(-c4ccccc4)nc(-c4ccccc4)n3)cc1C21CCCCC1 ; N#Cc1ccc2c(c1)C1(CCCCC1)c1cc(-c3cncc(-c4nc(-c5ccccc5)nc(-c5ccccc5)n4)c3)ccc1-2]

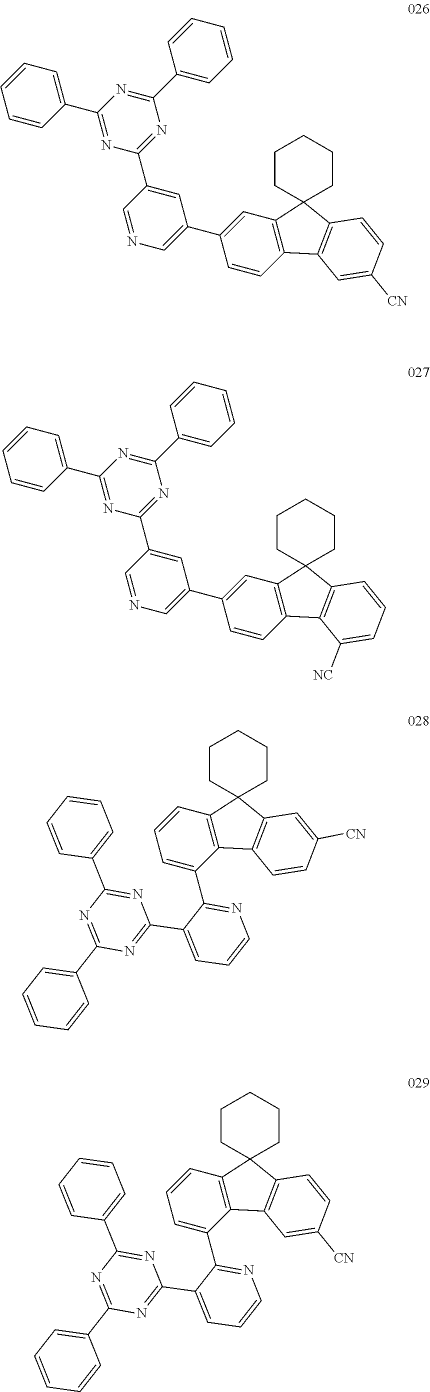 [SMILES: N#Cc1ccc2c(c1)-c1ccc(-c3cncc(-c4nc(-c5ccccc5)nc(-c5ccccc5)n4)c3)cc1C21CCCCC1 ; N#Cc1cccc2c1-c1ccc(-c3cncc(-c4nc(-c5ccccc5)nc(-c5ccccc5)n4)c3)cc1C21CCCCC1 ; N#Cc1ccc2c(c1)C1(CCCCC1)c1cccc(-c3ncccc3-c3nc(-c4ccccc4)nc(-c4ccccc4)n3)c1-2 ; N#Cc1ccc2c(c1)-c1c(-c3ncccc3-c3nc(-c4ccccc4)nc(-c4ccccc4)n3)cccc1C21CCCCC1]

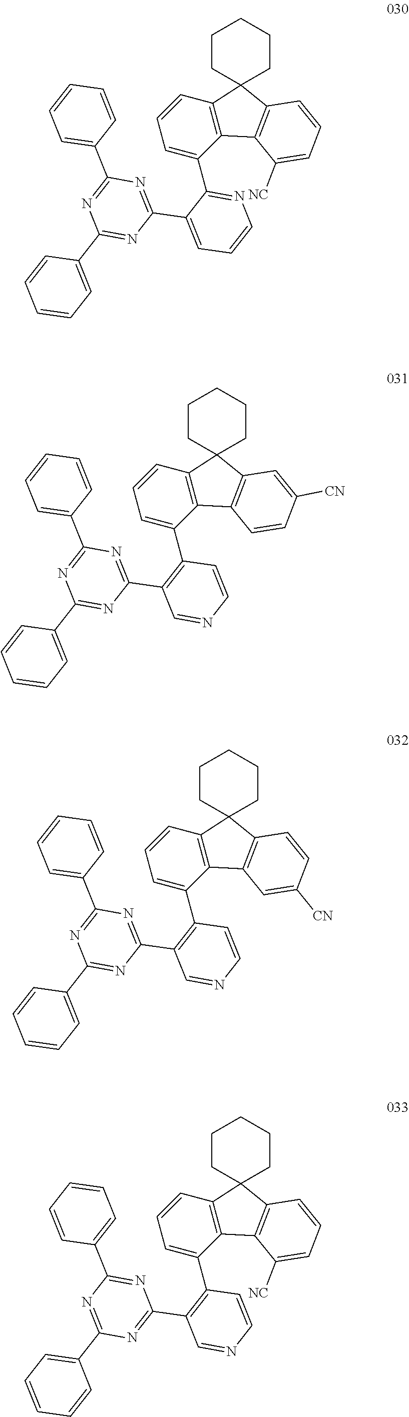 [SMILES: N#Cc1cccc2c1-c1c(-c3ncccc3-c3nc(-c4ccccc4)nc(-c4ccccc4)n3)cccc1C21CCCCC1 ; N#Cc1ccc2c(c1)C1(CCCCC1)c1cccc(-c3ccncc3-c3nc(-c4ccccc4)nc(-c4ccccc4)n3)c1-2 ; N#Cc1ccc2c(c1)-c1c(-c3ccncc3-c3nc(-c4ccccc4)nc(-c4ccccc4)n3)cccc1C21CCCCC1 ; N#Cc1cccc2c1-c1c(-c3ccncc3-c3nc(-c4ccccc4)nc(-c4ccccc4)n3)cccc1C21CCCCC1]

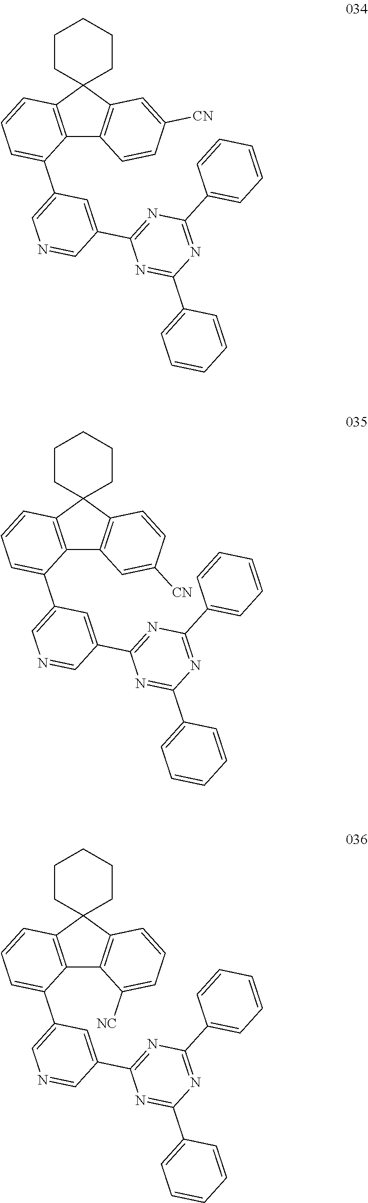 [SMILES: N#Cc1ccc2c(c1)C1(CCCCC1)c1cccc(-c3cncc(-c4nc(-c5ccccc5)nc(-c5ccccc5)n4)c3)c1-2 ; N#Cc1ccc2c(c1)-c1c(-c3cncc(-c4nc(-c5ccccc5)nc(-c5ccccc5)n4)c3)cccc1C21CCCCC1 ; N#Cc1cccc2c1-c1c(-c3cncc(-c4nc(-c5ccccc5)nc(-c5ccccc5)n4)c3)cccc1C21CCCCC1]

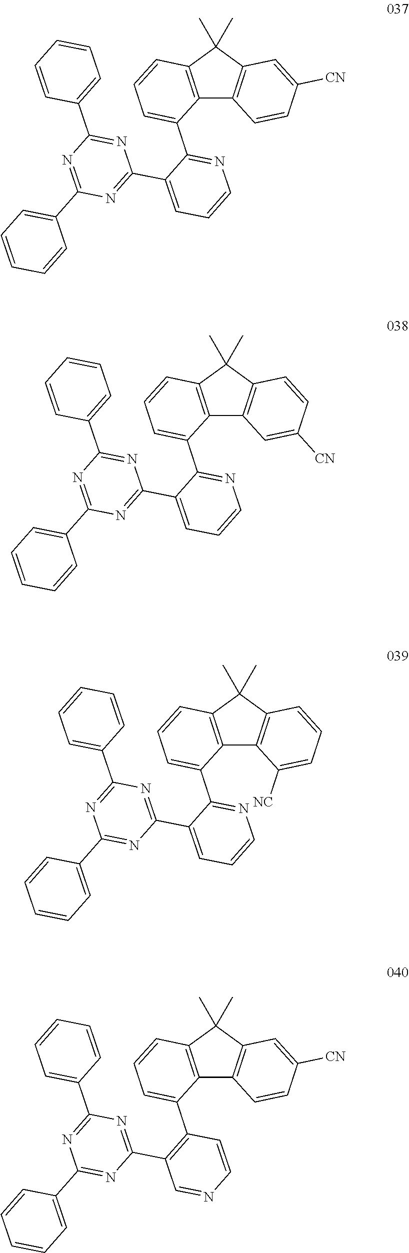 [SMILES: CC1(C)c2cc(C#N)ccc2-c2c(-c3ncccc3-c3nc(-c4ccccc4)nc(-c4ccccc4)n3)cccc21 ; CC1(C)c2ccc(C#N)cc2-c2c(-c3ncccc3-c3nc(-c4ccccc4)nc(-c4ccccc4)n3)cccc21 ; CC1(C)c2cccc(C#N)c2-c2c(-c3ncccc3-c3nc(-c4ccccc4)nc(-c4ccccc4)n3)cccc21 ; CC1(C)c2cc(C#N)ccc2-c2c(-c3ccncc3-c3nc(-c4ccccc4)nc(-c4ccccc4)n3)cccc21]

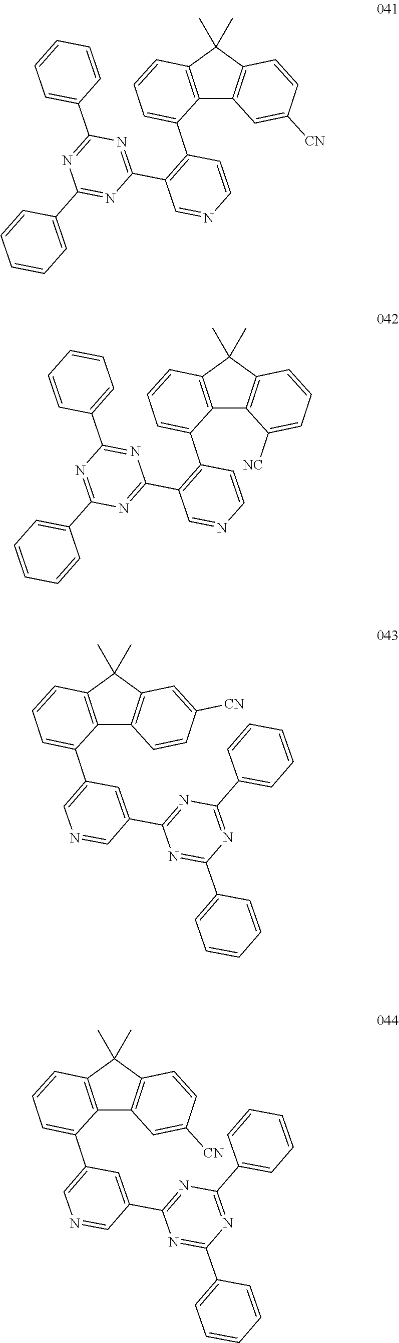 [SMILES: CC1(C)c2ccc(C#N)cc2-c2c(-c3ccncc3-c3nc(-c4ccccc4)nc(-c4ccccc4)n3)cccc21 ; CC1(C)c2cccc(C#N)c2-c2c(-c3ccncc3-c3nc(-c4ccccc4)nc(-c4ccccc4)n3)cccc21 ; CC1(C)c2cc(C#N)ccc2-c2c(-c3cncc(-c4nc(-c5ccccc5)nc(-c5ccccc5)n4)c3)cccc21 ; CC1(C)c2ccc(C#N)cc2-c2c(-c3cncc(-c4nc(-c5ccccc5)nc(-c5ccccc5)n4)c3)cccc21]

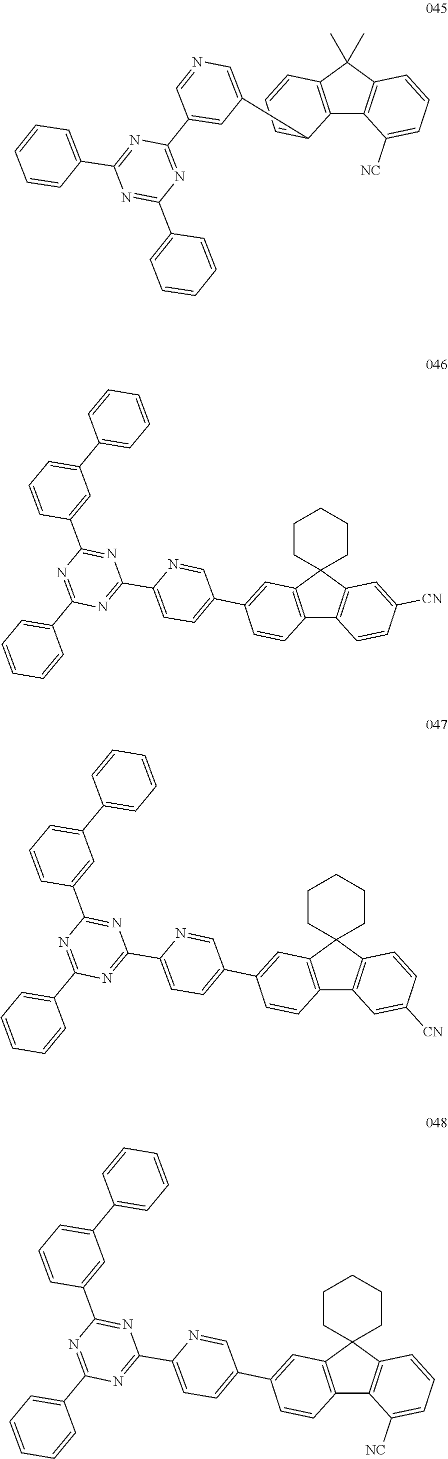 [SMILES: CC1(C)c2cccc(C#N)c2-c2c(-c3cncc(-c4nc(-c5ccccc5)nc(-c5ccccc5)n4)c3)cccc21 ; N#Cc1ccc2c(c1)C1(CCCCC1)c1cc(-c3ccc(-c4nc(-c5ccccc5)nc(-c5cccc(-c6ccccc6)c5)n4)nc3)ccc1-2 ; N#Cc1ccc2c(c1)-c1ccc(-c3ccc(-c4nc(-c5ccccc5)nc(-c5cccc(-c6ccccc6)c5)n4)nc3)cc1C21CCCCC1 ; N#Cc1cccc2c1-c1ccc(-c3ccc(-c4nc(-c5ccccc5)nc(-c5cccc(-c6ccccc6)c5)n4)nc3)cc1C21CCCCC1]

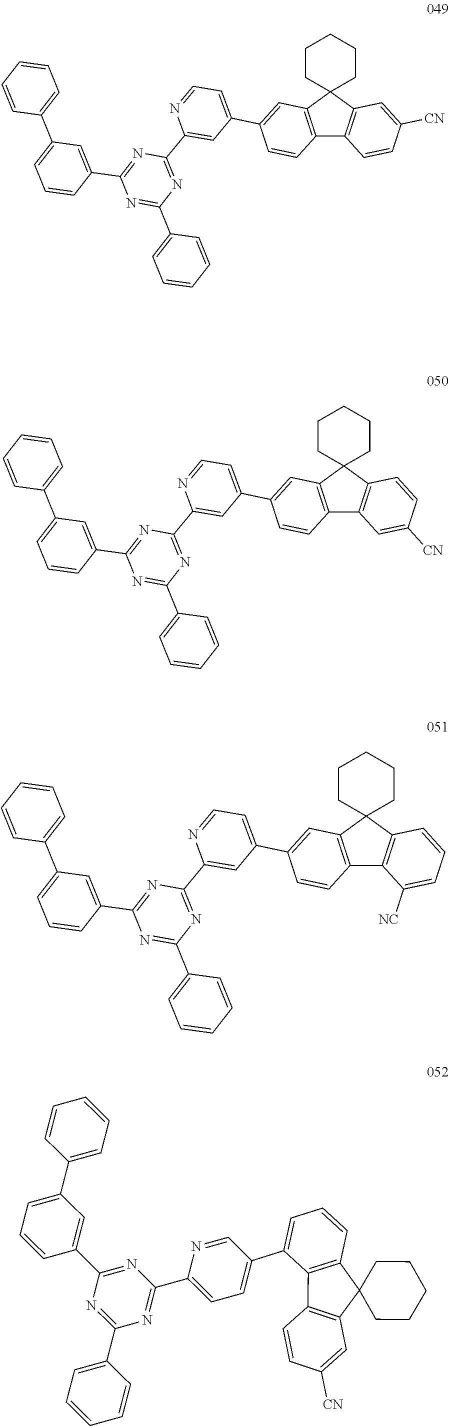 [SMILES: N#Cc1ccc2c(c1)C1(CCCCC1)c1cc(-c3ccnc(-c4nc(-c5ccccc5)nc(-c5cccc(-c6ccccc6)c5)n4)c3)ccc1-2 ; N#Cc1ccc2c(c1)-c1ccc(-c3ccnc(-c4nc(-c5ccccc5)nc(-c5cccc(-c6ccccc6)c5)n4)c3)cc1C21CCCCC1 ; N#Cc1cccc2c1-c1ccc(-c3ccnc(-c4nc(-c5ccccc5)nc(-c5cccc(-c6ccccc6)c5)n4)c3)cc1C21CCCCC1 ; N#Cc1ccc2c(c1)C1(CCCCC1)c1cccc(-c3ccc(-c4nc(-c5ccccc5)nc(-c5cccc(-c6ccccc6)c5)n4)nc3)c1-2]

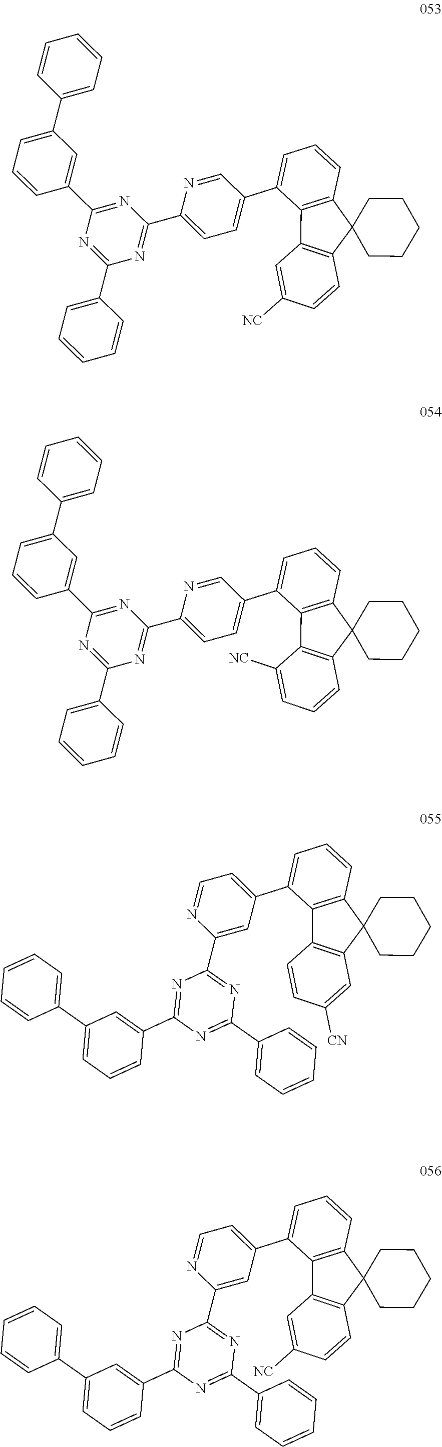 [SMILES: N#Cc1ccc2c(c1)-c1c(-c3ccc(-c4nc(-c5ccccc5)nc(-c5cccc(-c6ccccc6)c5)n4)nc3)cccc1C21CCCCC1 ; N#Cc1cccc2c1-c1c(-c3ccc(-c4nc(-c5ccccc5)nc(-c5cccc(-c6ccccc6)c5)n4)nc3)cccc1C21CCCCC1 ; N#Cc1ccc2c(c1)C1(CCCCC1)c1cccc(-c3ccnc(-c4nc(-c5ccccc5)nc(-c5cccc(-c6ccccc6)c5)n4)c3)c1-2 ; N#Cc1ccc2c(c1)-c1c(-c3ccnc(-c4nc(-c5ccccc5)nc(-c5cccc(-c6ccccc6)c5)n4)c3)cccc1C21CCCCC1]

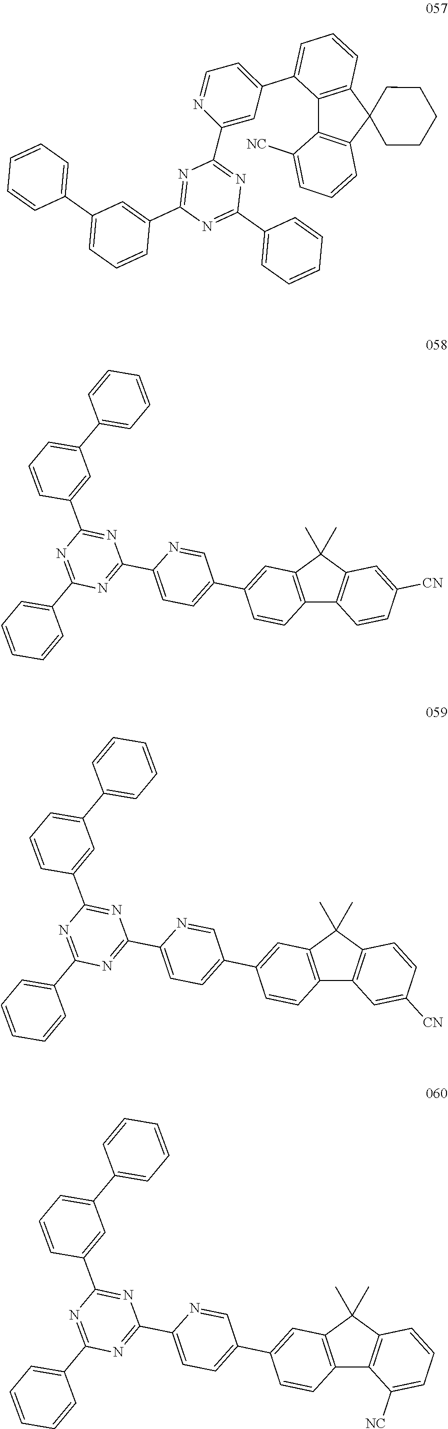 [SMILES: N#Cc1cccc2c1-c1c(-c3ccnc(-c4nc(-c5ccccc5)nc(-c5cccc(-c6ccccc6)c5)n4)c3)cccc1C21CCCCC1 ; CC1(C)c2cc(C#N)ccc2-c2ccc(-c3ccc(-c4nc(-c5ccccc5)nc(-c5cccc(-c6ccccc6)c5)n4)nc3)cc21 ; CC1(C)c2ccc(C#N)cc2-c2ccc(-c3ccc(-c4nc(-c5ccccc5)nc(-c5cccc(-c6ccccc6)c5)n4)nc3)cc21 ; CC1(C)c2cc(-c3ccc(-c4nc(-c5ccccc5)nc(-c5cccc(-c6ccccc6)c5)n4)nc3)ccc2-c2c(C#N)cccc21]

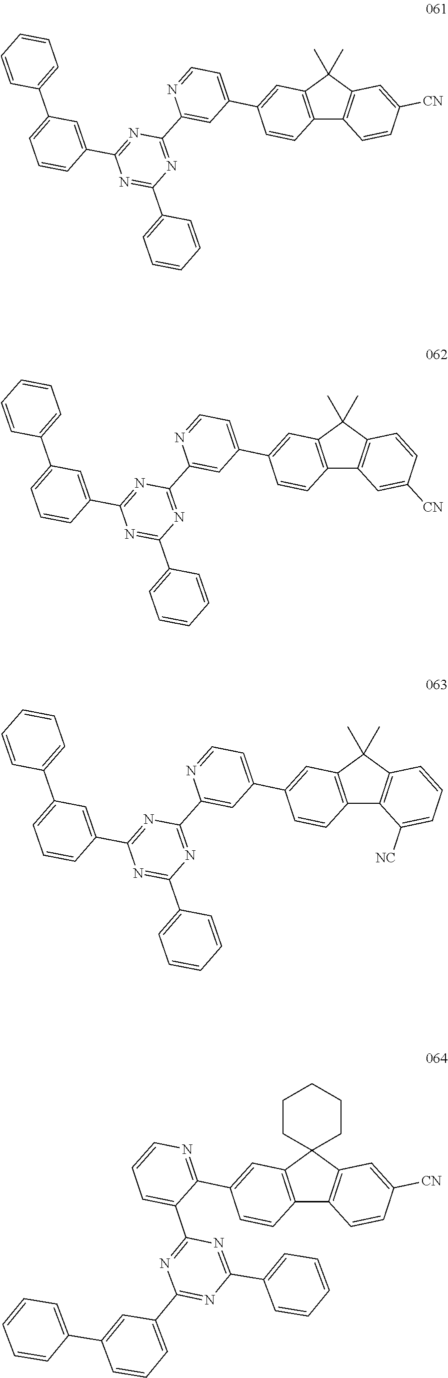 [SMILES: CC1(C)c2cc(C#N)ccc2-c2ccc(-c3ccnc(-c4nc(-c5ccccc5)nc(-c5cccc(-c6ccccc6)c5)n4)c3)cc21 ; CC1(C)c2ccc(C#N)cc2-c2ccc(-c3ccnc(-c4nc(-c5ccccc5)nc(-c5cccc(-c6ccccc6)c5)n4)c3)cc21 ; CC1(C)c2cc(-c3ccnc(-c4nc(-c5ccccc5)nc(-c5cccc(-c6ccccc6)c5)n4)c3)ccc2-c2c(C#N)cccc21 ; N#Cc1ccc2c(c1)C1(CCCCC1)c1cc(-c3ncccc3-c3nc(-c4ccccc4)nc(-c4cccc(-c5ccccc5)c4)n3)ccc1-2]

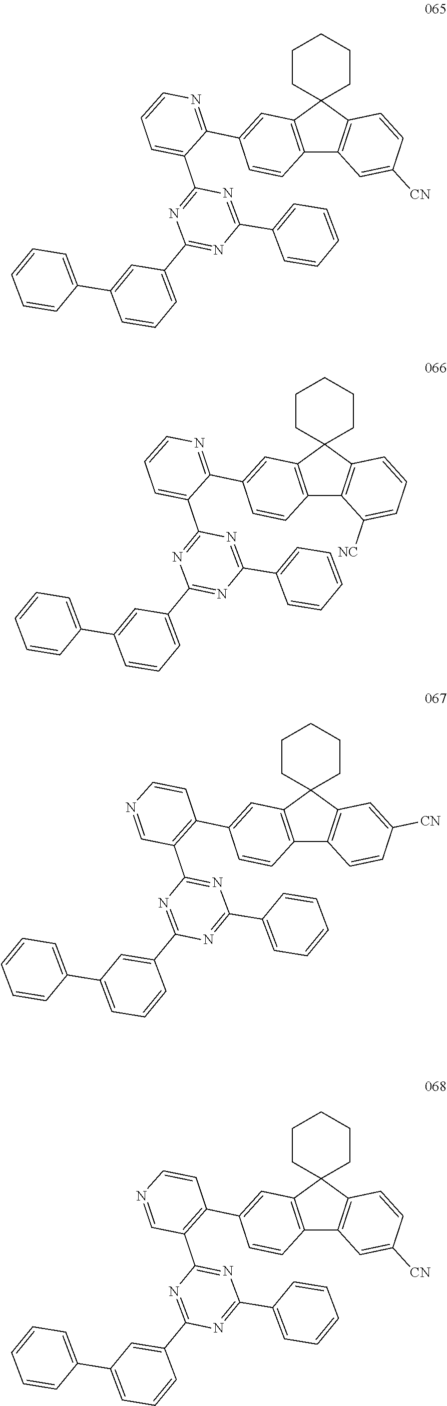 [SMILES: N#Cc1ccc2c(c1)-c1ccc(-c3ncccc3-c3nc(-c4ccccc4)nc(-c4cccc(-c5ccccc5)c4)n3)cc1C21CCCCC1 ; N#Cc1cccc2c1-c1ccc(-c3ncccc3-c3nc(-c4ccccc4)nc(-c4cccc(-c5ccccc5)c4)n3)cc1C21CCCCC1 ; N#Cc1ccc2c(c1)C1(CCCCC1)c1cc(-c3ccncc3-c3nc(-c4ccccc4)nc(-c4cccc(-c5ccccc5)c4)n3)ccc1-2 ; N#Cc1ccc2c(c1)-c1ccc(-c3ccncc3-c3nc(-c4ccccc4)nc(-c4cccc(-c5ccccc5)c4)n3)cc1C21CCCCC1]

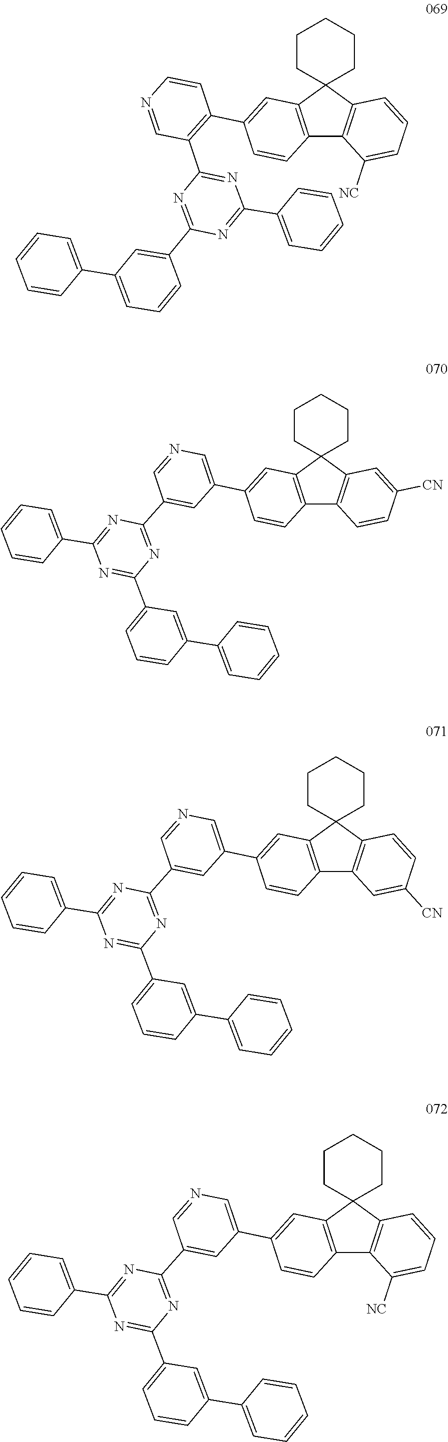 [SMILES: N#Cc1cccc2c1-c1ccc(-c3ccncc3-c3nc(-c4ccccc4)nc(-c4cccc(-c5ccccc5)c4)n3)cc1C21CCCCC1 ; N#Cc1ccc2c(c1)C1(CCCCC1)c1cc(-c3cncc(-c4nc(-c5ccccc5)nc(-c5cccc(-c6ccccc6)c5)n4)c3)ccc1-2 ; N#Cc1ccc2c(c1)-c1ccc(-c3cncc(-c4nc(-c5ccccc5)nc(-c5cccc(-c6ccccc6)c5)n4)c3)cc1C21CCCCC1 ; N#Cc1cccc2c1-c1ccc(-c3cncc(-c4nc(-c5ccccc5)nc(-c5cccc(-c6ccccc6)c5)n4)c3)cc1C21CCCCC1]

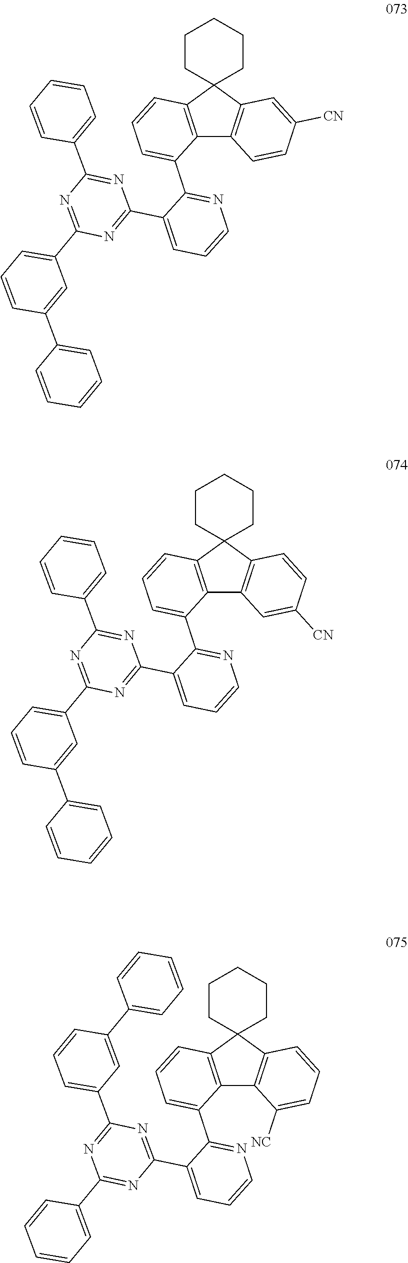 [SMILES: N#Cc1ccc2c(c1)C1(CCCCC1)c1cccc(-c3ncccc3-c3nc(-c4ccccc4)nc(-c4cccc(-c5ccccc5)c4)n3)c1-2 ; N#Cc1ccc2c(c1)-c1c(-c3ncccc3-c3nc(-c4ccccc4)nc(-c4cccc(-c5ccccc5)c4)n3)cccc1C21CCCCC1 ; N#Cc1cccc2c1-c1c(-c3ncccc3-c3nc(-c4ccccc4)nc(-c4cccc(-c5ccccc5)c4)n3)cccc1C21CCCCC1]

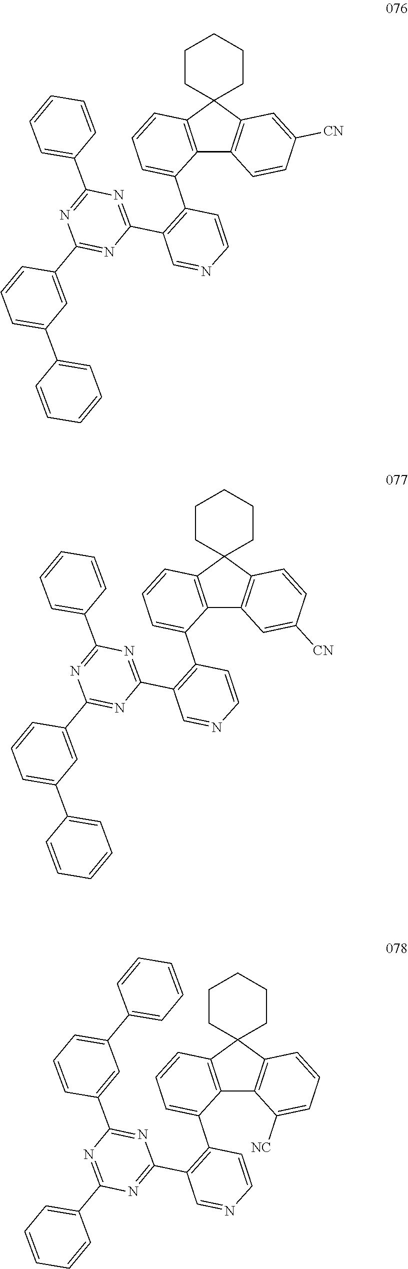 [SMILES: N#Cc1ccc2c(c1)C1(CCCCC1)c1cccc(-c3ccncc3-c3nc(-c4ccccc4)nc(-c4cccc(-c5ccccc5)c4)n3)c1-2 ; N#Cc1ccc2c(c1)-c1c(-c3ccncc3-c3nc(-c4ccccc4)nc(-c4cccc(-c5ccccc5)c4)n3)cccc1C21CCCCC1 ; N#Cc1cccc2c1-c1c(-c3ccncc3-c3nc(-c4ccccc4)nc(-c4cccc(-c5ccccc5)c4)n3)cccc1C21CCCCC1]

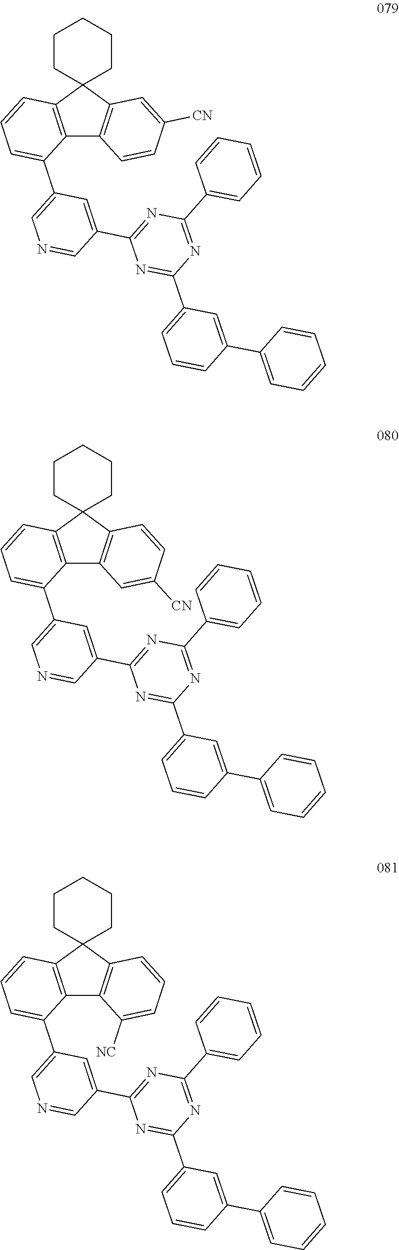 [SMILES: N#Cc1ccc2c(c1)C1(CCCCC1)c1cccc(-c3cncc(-c4nc(-c5ccccc5)nc(-c5cccc(-c6ccccc6)c5)n4)c3)c1-2 ; N#Cc1ccc2c(c1)-c1c(-c3cncc(-c4nc(-c5ccccc5)nc(-c5cccc(-c6ccccc6)c5)n4)c3)cccc1C21CCCCC1 ; N#Cc1cccc2c1-c1c(-c3cncc(-c4nc(-c5ccccc5)nc(-c5cccc(-c6ccccc6)c5)n4)c3)cccc1C21CCCCC1]

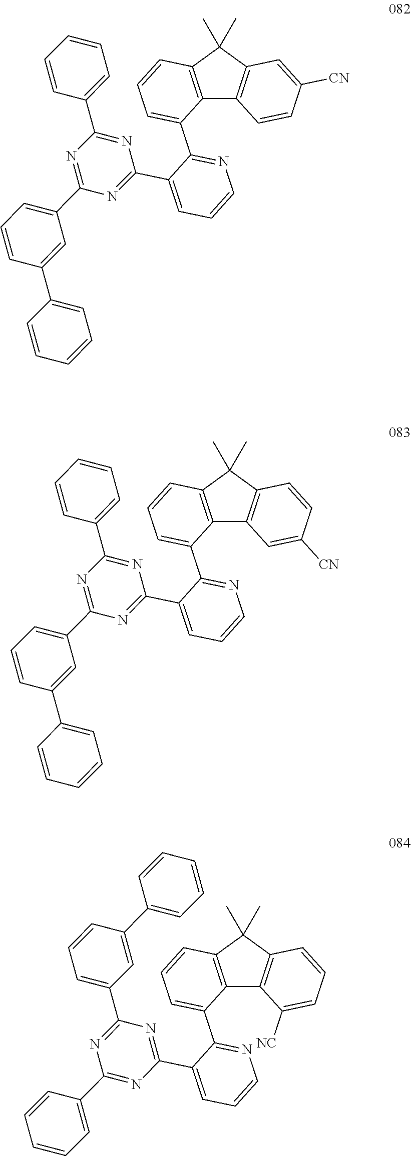 [SMILES: CC1(C)c2cc(C#N)ccc2-c2c(-c3ncccc3-c3nc(-c4ccccc4)nc(-c4cccc(-c5ccccc5)c4)n3)cccc21 ; CC1(C)c2ccc(C#N)cc2-c2c(-c3ncccc3-c3nc(-c4ccccc4)nc(-c4cccc(-c5ccccc5)c4)n3)cccc21 ; CC1(C)c2cccc(C#N)c2-c2c(-c3ncccc3-c3nc(-c4ccccc4)nc(-c4cccc(-c5ccccc5)c4)n3)cccc21]

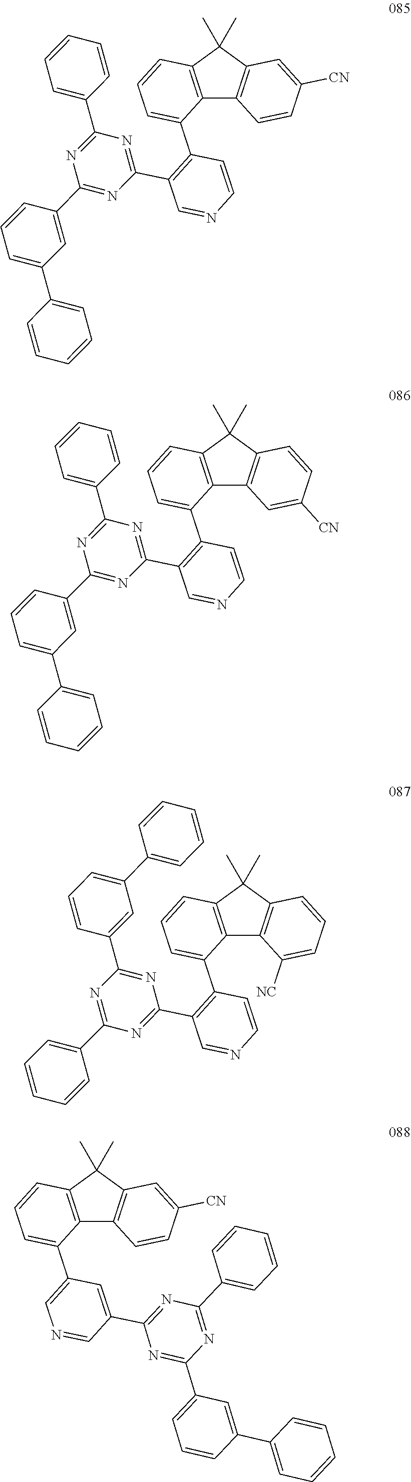

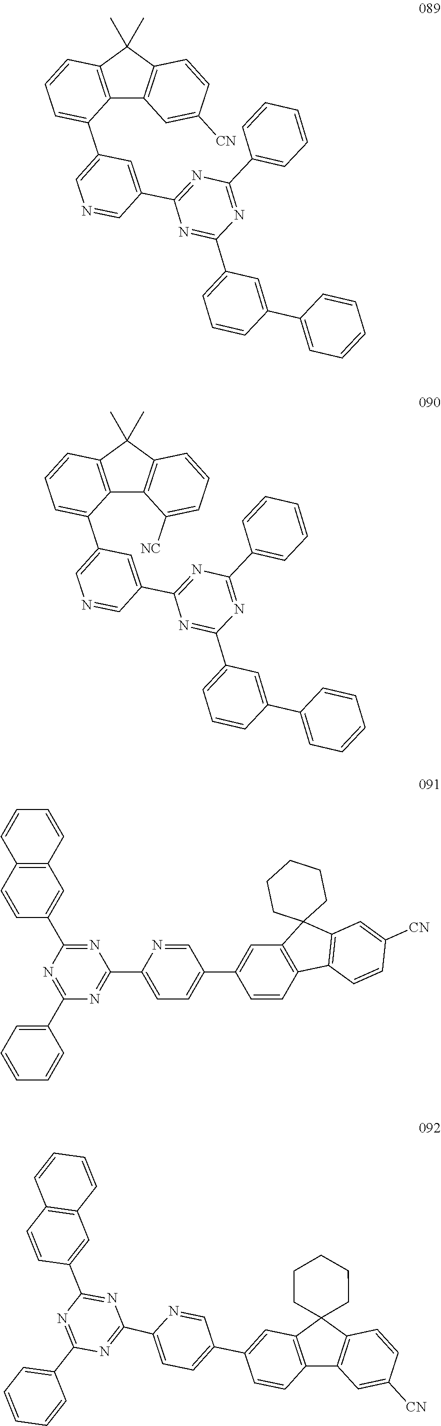 [SMILES: CC1(C)c2ccc(C#N)cc2-c2c(-c3cncc(-c4nc(-c5ccccc5)nc(-c5cccc(-c6ccccc6)c5)n4)c3)cccc21 ; CC1(C)c2cccc(C#N)c2-c2c(-c3cncc(-c4nc(-c5ccccc5)nc(-c5cccc(-c6ccccc6)c5)n4)c3)cccc21 ; N#Cc1ccc2c(c1)C1(CCCCC1)c1cc(-c3ccc(-c4nc(-c5ccccc5)nc(-c5ccc6ccccc6c5)n4)nc3)ccc1-2 ; N#Cc1ccc2c(c1)-c1ccc(-c3ccc(-c4nc(-c5ccccc5)nc(-c5ccc6ccccc6c5)n4)nc3)cc1C21CCCCC1]

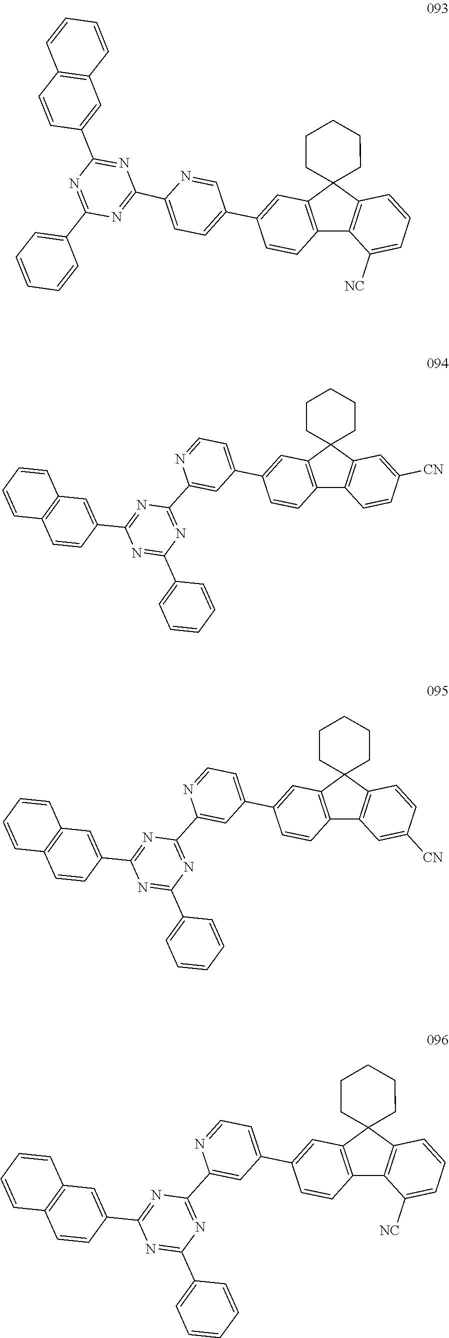 [SMILES: N#Cc1cccc2c1-c1ccc(-c3ccc(-c4nc(-c5ccccc5)nc(-c5ccc6ccccc6c5)n4)nc3)cc1C21CCCCC1 ; N#Cc1ccc2c(c1)C1(CCCCC1)c1cc(-c3ccnc(-c4nc(-c5ccccc5)nc(-c5ccc6ccccc6c5)n4)c3)ccc1-2 ; N#Cc1ccc2c(c1)-c1ccc(-c3ccnc(-c4nc(-c5ccccc5)nc(-c5ccc6ccccc6c5)n4)c3)cc1C21CCCCC1 ; N#Cc1cccc2c1-c1ccc(-c3ccnc(-c4nc(-c5ccccc5)nc(-c5ccc6ccccc6c5)n4)c3)cc1C21CCCCC1]

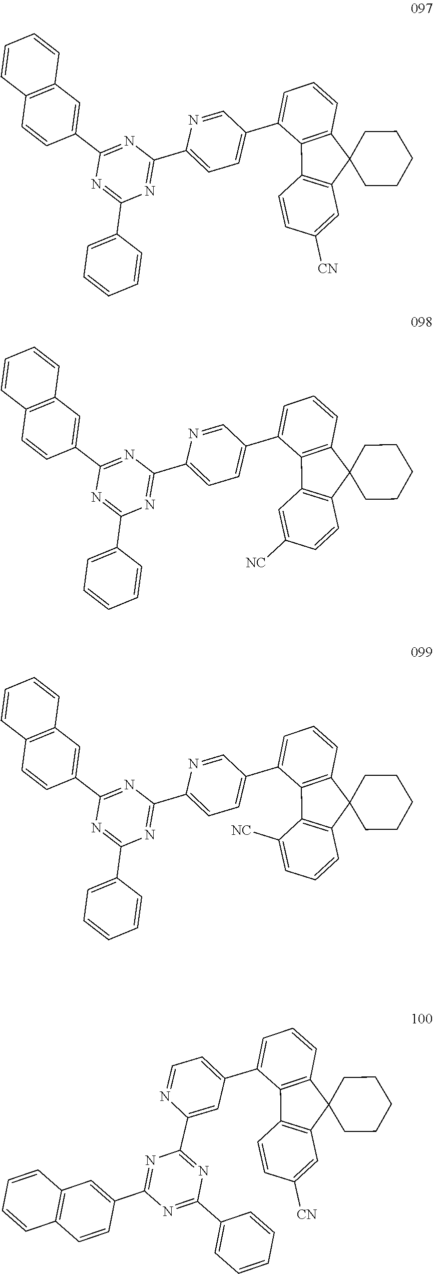 [SMILES: N#Cc1ccc2c(c1)C1(CCCCC1)c1cccc(-c3ccc(-c4nc(-c5ccccc5)nc(-c5ccc6ccccc6c5)n4)nc3)c1-2 ; N#Cc1ccc2c(c1)-c1c(-c3ccc(-c4nc(-c5ccccc5)nc(-c5ccc6ccccc6c5)n4)nc3)cccc1C21CCCCC1 ; N#Cc1cccc2c1-c1c(-c3ccc(-c4nc(-c5ccccc5)nc(-c5ccc6ccccc6c5)n4)nc3)cccc1C21CCCCC1 ; N#Cc1ccc2c(c1)C1(CCCCC1)c1cccc(-c3ccnc(-c4nc(-c5ccccc5)nc(-c5ccc6ccccc6c5)n4)c3)c1-2]

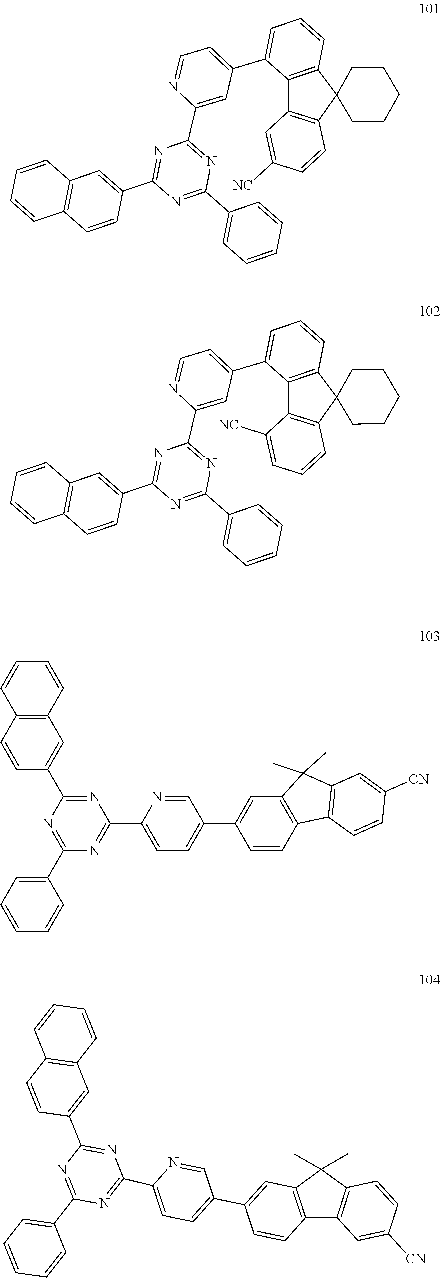 [SMILES: N#Cc1ccc2c(c1)-c1c(-c3ccnc(-c4nc(-c5ccccc5)nc(-c5ccc6ccccc6c5)n4)c3)cccc1C21CCCCC1 ; N#Cc1cccc2c1-c1c(-c3ccnc(-c4nc(-c5ccccc5)nc(-c5ccc6ccccc6c5)n4)c3)cccc1C21CCCCC1 ; CC1(C)c2cc(C#N)ccc2-c2ccc(-c3ccc(-c4nc(-c5ccccc5)nc(-c5ccc6ccccc6c5)n4)nc3)cc21 ; CC1(C)c2ccc(C#N)cc2-c2ccc(-c3ccc(-c4nc(-c5ccccc5)nc(-c5ccc6ccccc6c5)n4)nc3)cc21]

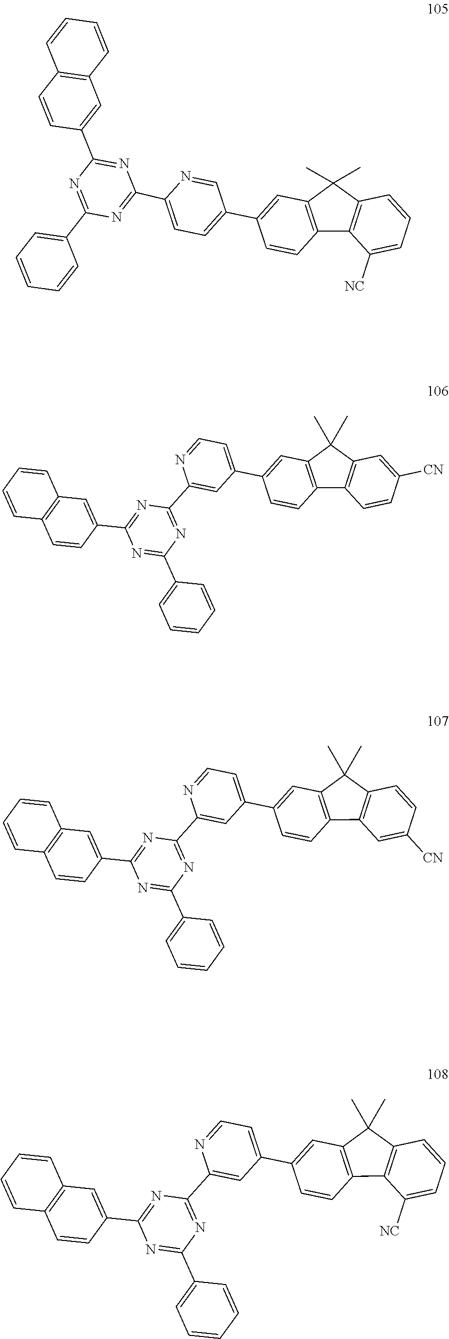 [SMILES: CC1(C)c2cc(-c3ccc(-c4nc(-c5ccccc5)nc(-c5ccc6ccccc6c5)n4)nc3)ccc2-c2c(C#N)cccc21 ; CC1(C)c2cc(C#N)ccc2-c2ccc(-c3ccnc(-c4nc(-c5ccccc5)nc(-c5ccc6ccccc6c5)n4)c3)cc21 ; CC1(C)c2ccc(C#N)cc2-c2ccc(-c3ccnc(-c4nc(-c5ccccc5)nc(-c5ccc6ccccc6c5)n4)c3)cc21 ; CC1(C)c2cc(-c3ccnc(-c4nc(-c5ccccc5)nc(-c5ccc6ccccc6c5)n4)c3)ccc2-c2c(C#N)cccc21]

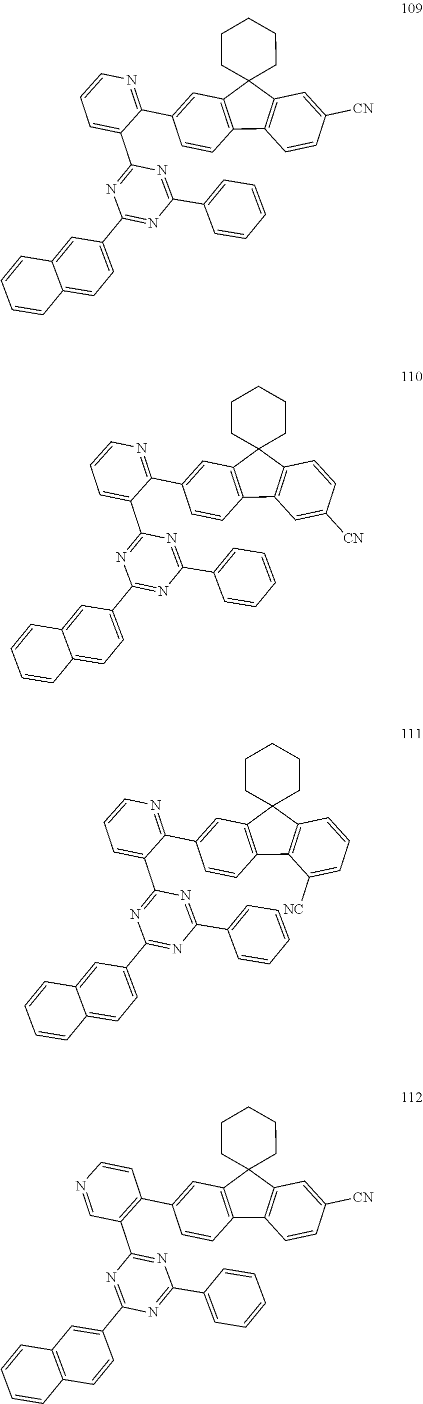 [SMILES: N#Cc1ccc2c(c1)C1(CCCCC1)c1cc(-c3ncccc3-c3nc(-c4ccccc4)nc(-c4ccc5ccccc5c4)n3)ccc1-2 ; N#Cc1ccc2c(c1)-c1ccc(-c3ncccc3-c3nc(-c4ccccc4)nc(-c4ccc5ccccc5c4)n3)cc1C21CCCCC1 ; N#Cc1cccc2c1-c1ccc(-c3ncccc3-c3nc(-c4ccccc4)nc(-c4ccc5ccccc5c4)n3)cc1C21CCCCC1 ; N#Cc1ccc2c(c1)C1(CCCCC1)c1cc(-c3ccncc3-c3nc(-c4ccccc4)nc(-c4ccc5ccccc5c4)n3)ccc1-2]

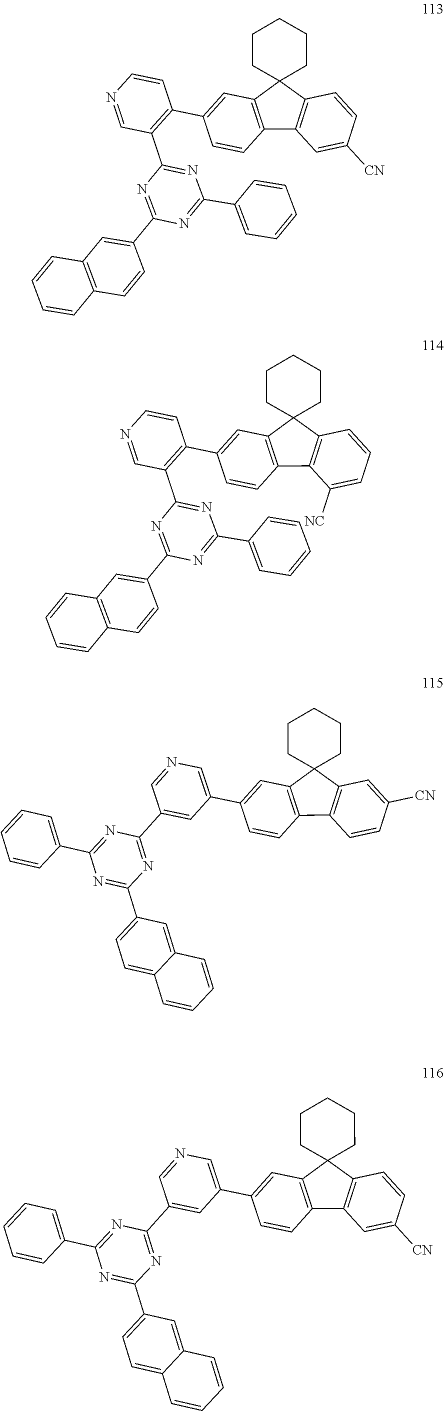 [SMILES: N#Cc1ccc2c(c1)-c1ccc(-c3ccncc3-c3nc(-c4ccccc4)nc(-c4ccc5ccccc5c4)n3)cc1C21CCCCC1 ; N#Cc1cccc2c1-c1ccc(-c3ccncc3-c3nc(-c4ccccc4)nc(-c4ccc5ccccc5c4)n3)cc1C21CCCCC1 ; N#Cc1ccc2c(c1)C1(CCCCC1)c1cc(-c3cncc(-c4nc(-c5ccccc5)nc(-c5ccc6ccccc6c5)n4)c3)ccc1-2 ; N#Cc1ccc2c(c1)-c1ccc(-c3cncc(-c4nc(-c5ccccc5)nc(-c5ccc6ccccc6c5)n4)c3)cc1C21CCCCC1]

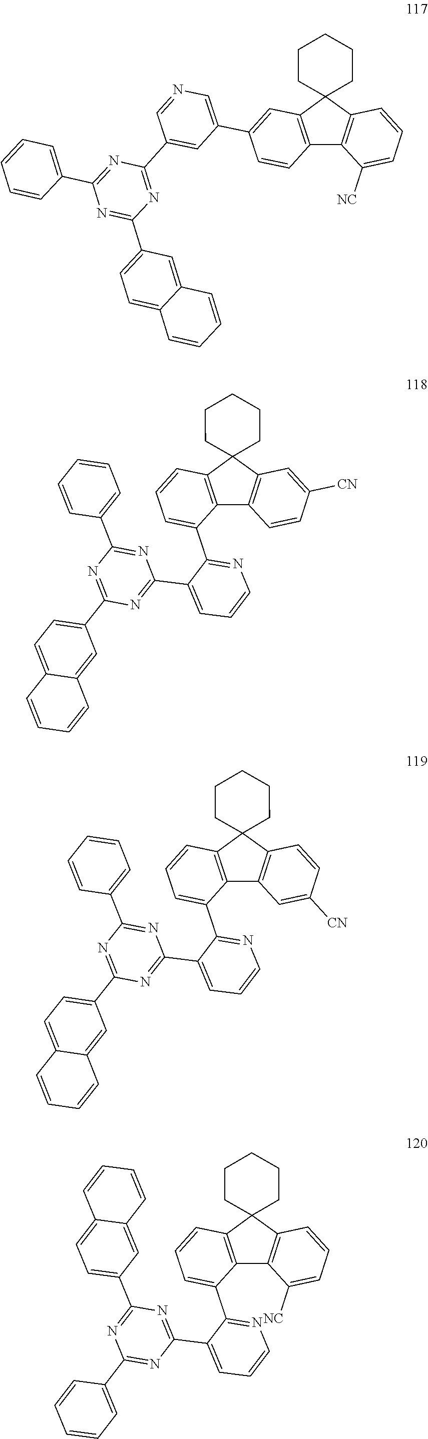 [SMILES: N#Cc1cccc2c1-c1ccc(-c3cncc(-c4nc(-c5ccccc5)nc(-c5ccc6ccccc6c5)n4)c3)cc1C21CCCCC1 ; N#Cc1ccc2c(c1)C1(CCCCC1)c1cccc(-c3ncccc3-c3nc(-c4ccccc4)nc(-c4ccc5ccccc5c4)n3)c1-2 ; N#Cc1ccc2c(c1)-c1c(-c3ncccc3-c3nc(-c4ccccc4)nc(-c4ccc5ccccc5c4)n3)cccc1C21CCCCC1 ; N#Cc1cccc2c1-c1c(-c3ncccc3-c3nc(-c4ccccc4)nc(-c4ccc5ccccc5c4)n3)cccc1C21CCCCC1]

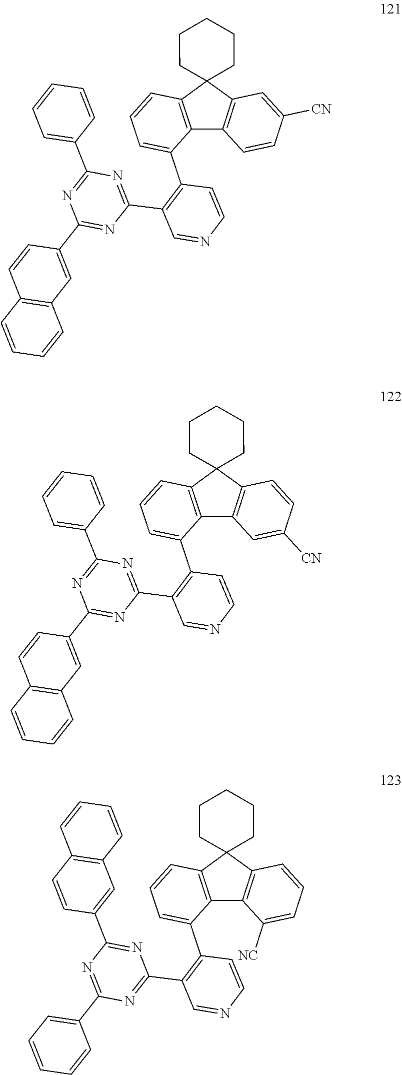 [SMILES: N#Cc1ccc2c(c1)C1(CCCCC1)c1cccc(-c3ccncc3-c3nc(-c4ccccc4)nc(-c4ccc5ccccc5c4)n3)c1-2 ; N#Cc1ccc2c(c1)-c1c(-c3ccncc3-c3nc(-c4ccccc4)nc(-c4ccc5ccccc5c4)n3)cccc1C21CCCCC1 ; N#Cc1cccc2c1-c1c(-c3ccncc3-c3nc(-c4ccccc4)nc(-c4ccc5ccccc5c4)n3)cccc1C21CCCCC1]

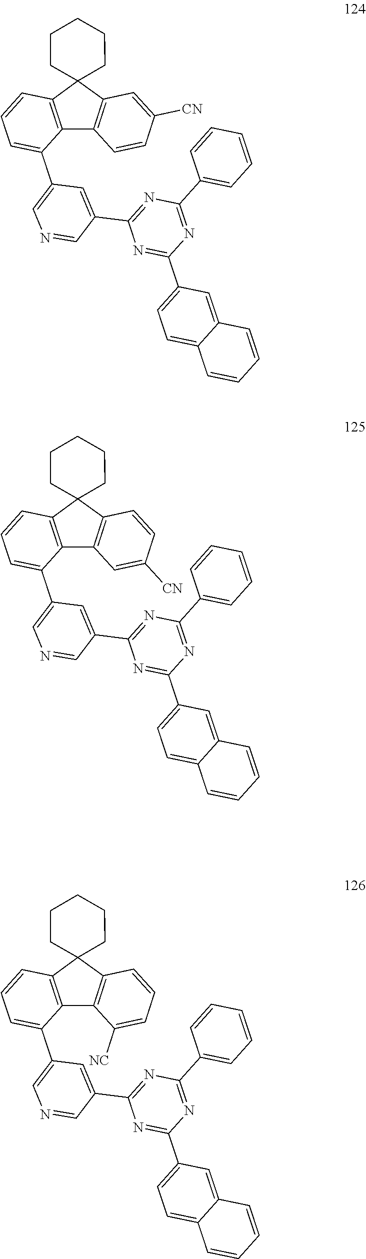 [SMILES: N#Cc1ccc2c(c1)C1(CCCCC1)c1cccc(-c3cncc(-c4nc(-c5ccccc5)nc(-c5ccc6ccccc6c5)n4)c3)c1-2 ; N#Cc1ccc2c(c1)-c1c(-c3cncc(-c4nc(-c5ccccc5)nc(-c5ccc6ccccc6c5)n4)c3)cccc1C21CCCCC1 ; N#Cc1cccc2c1-c1c(-c3cncc(-c4nc(-c5ccccc5)nc(-c5ccc6ccccc6c5)n4)c3)cccc1C21CCCCC1]

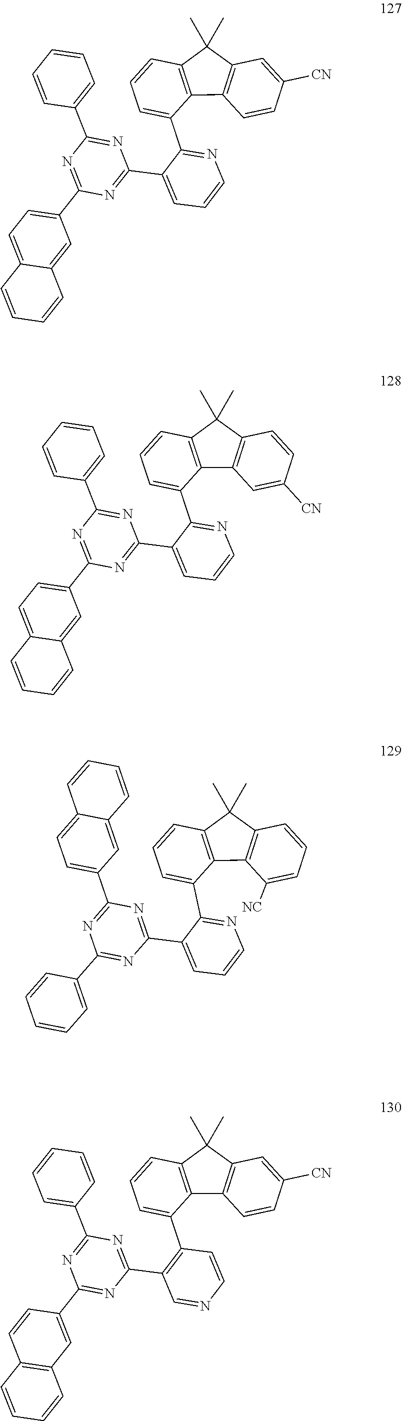 [SMILES: CC1(C)c2cc(C#N)ccc2-c2c(-c3ncccc3-c3nc(-c4ccccc4)nc(-c4ccc5ccccc5c4)n3)cccc21 ; CC1(C)c2ccc(C#N)cc2-c2c(-c3ncccc3-c3nc(-c4ccccc4)nc(-c4ccc5ccccc5c4)n3)cccc21 ; CC1(C)c2cccc(C#N)c2-c2c(-c3ncccc3-c3nc(-c4ccccc4)nc(-c4ccc5ccccc5c4)n3)cccc21 ; CC1(C)c2cc(C#N)ccc2-c2c(-c3ccncc3-c3nc(-c4ccccc4)nc(-c4ccc5ccccc5c4)n3)cccc21]

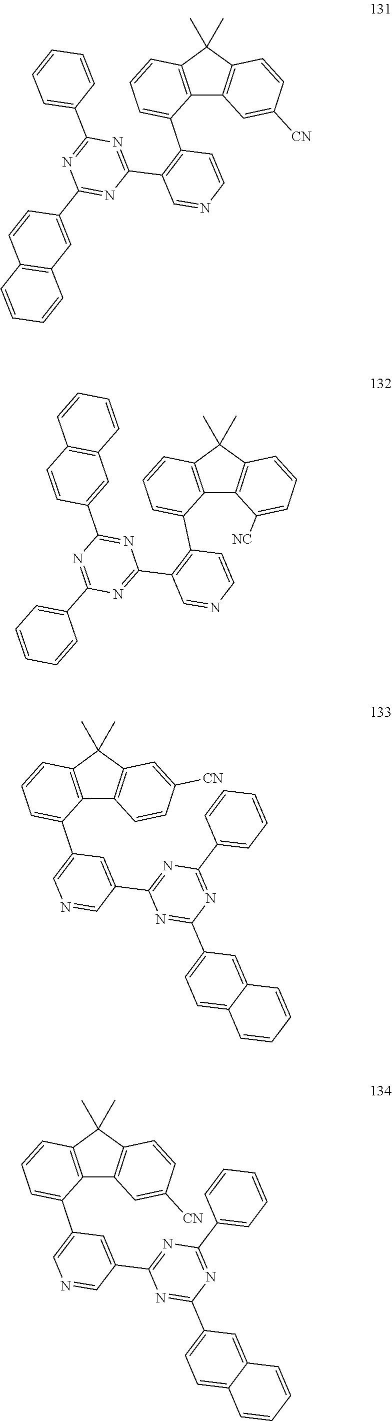 [SMILES: CC1(C)c2ccc(C#N)cc2-c2c(-c3ccncc3-c3nc(-c4ccccc4)nc(-c4ccc5ccccc5c4)n3)cccc21 ; CC1(C)c2cccc(C#N)c2-c2c(-c3ccncc3-c3nc(-c4ccccc4)nc(-c4ccc5ccccc5c4)n3)cccc21 ; CC1(C)c2cc(C#N)ccc2-c2c(-c3cncc(-c4nc(-c5ccccc5)nc(-c5ccc6ccccc6c5)n4)c3)cccc21 ; CC1(C)c2ccc(C#N)cc2-c2c(-c3cncc(-c4nc(-c5ccccc5)nc(-c5ccc6ccccc6c5)n4)c3)cccc21]

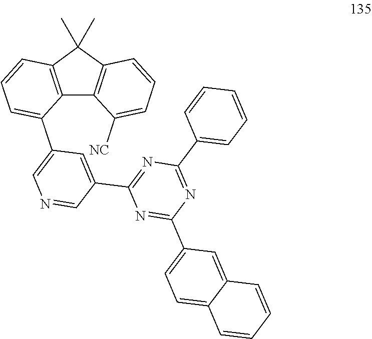 [SMILES: CC1(C)c2cccc(C#N)c2-c2c(-c3cncc(-c4nc(-c5ccccc5)nc(-c5ccc6ccccc6c5)n4)c3)cccc21]

As used herein, “the number of nuclear atoms” means the number of ring atoms constituting a ring structure, and the nuclear atoms may mean carbon or a heteroatom selected from the group consisting of N, O, S and Se. For example, the number of nuclear atoms of pyridine means 6 including 5 C and 1 N constituting a pyridine ring.

As used herein, “alkyl” refers to a monovalent substituent derived from a linear or branched chain saturated hydrocarbon having 1 to 40 carbon atoms. Examples of such alkyl may include, but not limited to, methyl, ethyl, propyl, isobutyl, sec-butyl, pentyl, iso-amyl, hexyl or the like.

As used herein, “alkenyl” refers to a monovalent substituent derived from a C₂ to C₄₀ linear or branched chain unsaturated hydrocarbon, having at least one carbon-carbon double bond. Examples of such alkenyl may include, but not limited to, vinyl, allyl, isopropenyl, 2-butenyl or the like.

As used herein, “alkynyl” refers to a monovalent substituent derived from a C₂ to C₄₀ linear or branched chain unsaturated hydrocarbon, having at least one carbon-carbon triple bond. Examples of such alkynyl may include, but not limited to, ethynyl, 2-propynyl or the like.

As used herein, “aryl” refers to a monovalent substituent derived from a C₆ to C₄₀ aromatic hydrocarbon having a structure with a single ring or two or more rings combined with each other. In addition, a form in which two or more rings are pendant (e.g., simply attached) to or fused with each other may also be included. Examples of such aryl may include, but not limited to, phenyl, naphthyl, phenanthryl, anthryl or the like.

As used herein, “heteroaryl” refers to a monovalent substituent derived from a monoheterocyclic or polyheterocyclic aromatic hydrocarbon having 5 to 40 nuclear atoms. In such an embodiment, one or more carbons in the ring, preferably one to three carbons, are substituted with a heteroatom such as N, O, S or Se. In addition, a form in which two or more rings are pendant to or fused with each other may be included and a form fused with an aryl group may be included. Examples of such heteroaryl may include, but not limited to, a 6-membered monocyclic ring such as pyridyl, pyrazinyl, pyrimidinyl, pyridazinyl and triazinyl; a polycyclic ring such as phenoxathienyl, indolizinyl, indolyl, purinyl, quinolyl, benzothiazole, and carbazolyl; 2-furanyl; N-imidazolyl; 2-isoxazolyl; 2-pyridinyl; 2-pyrimidinyl or the like.

As used herein, “aryloxy” is a monovalent substituent represented by RO—, where R refers to a C₅ to C₄₀ aryl. Examples of such aryloxy may include, but not limited to, phenyloxy, naphthyloxy, diphenyloxy or the like.

As used herein, “alkyloxy” refers to a monovalent substituent represented by R′O—, where R′ refers to a C₁ to C₄₀ alkyl. Such alkyloxy may include a linear, branched or cyclic structure. Examples of such alkyloxy may include, but not limited to, methoxy, ethoxy, n-propoxy, 1-propoxy, t-butoxy, n-butoxy, pentoxy or the like.

As used herein, “arylamine” refers to amine substituted with a C₆ to C₄₀ aryl.

As used herein, “cycloalkyl” refers to a monovalent substituent derived from a C₃ to C₄₀ monocyclic or polycyclic non-aromatic hydrocarbon. Examples of such cycloalkyl may include, but not limited to, cyclopropyl, cyclopentyl, cyclohexyl, norbornyl, adamantine or the like.

As used herein, “heterocycloalkyl” refers to a monovalent substituent derived from a non-aromatic hydrocarbon having 3 to 40 nuclear atoms, where one or more carbons in the ring, preferably one to three carbons, are substituted with a heteroatom such as N, O, S or Se. Examples of such heterocycloalkyl may include, but not limited to, morpholine, piperazine or the like.

As used herein, “alkylsilyl” refers to silyl substituted with a C₁ to C₄₀ alkyl, and “arylsilyl” refers to silyl substituted with a C₅ to C₄₀ aryl.

As used herein, the term “fused ring (e.g., condensed ring)” refers to a condensed aliphatic ring, a condensed aromatic ring, a condensed heteroaliphatic ring, a condensed heteroaromatic ring, or a combination thereof.

### <Electron Transport Layer Material>

The present invention provides an electron transport layer including the compound represented by Chemical Formula 1.

The electron transport layer (ETL) serves to move electrons injected from a cathode to an adjacent layer, specifically a light emitting layer.

The compound represented by Chemical Formula 1 may be used alone as an electron transport layer (ETL) material, or may be used in combination with an electron transport layer material known in the art. It may preferably be used alone.

The electron transport layer material that may be used in combination with the compound of Chemical Formula 1 may include an electron transport material commonly known in the art. Non-limiting examples of applicable electron transport materials may include oxazole-based compounds, isoxazole-based compounds, triazole-based compounds, isothiazole-based compounds, oxadiazole-based compounds, thiadiazole-based compounds, perylene-based compounds, aluminum complexes (e.g., tris(8-quinolinolato)-aluminium (Alq₃), BAlq, SAlq, Almq₃, gallium complexes (e.g., Gaq′2OPiv, Gaq′2OAc, 2(Gaq′2)), etc. These may be used alone or two or more types thereof may be used in combination.

In the present invention, when the compound of Chemical Formula 1 and the electron transport layer material are used in combination, a mixing ratio thereof is not particularly limited, and may be appropriately adjusted within a range known in the art.

### <Auxiliary Electron Transport Layer Material>

In addition, the present invention provides an auxiliary electron transport layer including the compound represented by Chemical Formula 1.

The auxiliary electron transport layer is disposed between the light emitting layer and the electron transport layer and serves to substantially prevent diffusion of excitons or holes generated in the light emitting layer into the electron transport layer.

The compound represented by Chemical Formula 1 may be used alone as an auxiliary electron transport layer material, or may be combined with an electron transport layer material known in the art. It may preferably be used alone.

The auxiliary electron transport layer material that may be used in combination with the compound of Chemical Formula 1 includes an electron transport material commonly known in the art. For example, the auxiliary electron transport layer may include an oxadiazole derivative, a triazole derivative, a phenanthroline derivative (e.g., BCP), a heterocyclic derivative containing nitrogen, and the like.

In the present invention, when the compound of Chemical Formula 1 and the auxiliary electron transport layer material are used in combination, a mixing ratio thereof is not particularly limited, and may be appropriately adjusted within a range known in the art.

### <Organic Electroluminescent Device>

Another aspect of the present invention is directed to an organic electroluminescent device (“organic EL element”) including the compound represented by Chemical Formula 1.

More specifically, the organic EL device according to the present invention includes an anode (e.g., a positive electrode), a cathode (e.g., a negative electrode), and one or more organic layers disposed between the anode and the cathode, and at least one of the one or more organic layers includes the compound represented by Chemical Formula 1. In such an embodiment, the compound may be used alone or in combination of two or more kinds thereof.

The one or more organic layers may be any one or more of a hole injection layer, a hole transport layer, a light emitting layer, a light emitting auxiliary layer, a lifespan improvement layer, an electron transport layer, an auxiliary electron transport layer and an electron injection layer, and at least one of the organic layers may include the compound represented by Chemical Formula 1. Specifically, the organic layer including the compound represented by Chemical Formula 1 may preferably be a light emitting layer, a light emitting auxiliary layer, an electron transport layer, an auxiliary electron transport layer and/or a lifespan improvement layer, and more specifically, it may be preferable to be an electron transport layer or an auxiliary electron transport layer.

The light emitting layer of the organic EL device of the present invention includes a host material and a dopant material, and may include the compound of Chemical Formula 1 as a host material. In addition, the light emitting layer of the present invention may include, as a host, other compounds known in the art rather than or in addition to the compound of Chemical Formula 1.

When the compound represented by Chemical Formula 1 is included as a material for the light emitting layer of the organic EL element, preferably as a blue, green, or red phosphorescent host material, since a bonding force between holes and electrons in the light emitting layer is increased, the efficiency (luminescence efficiency and power efficiency), lifespan, luminance, driving voltage, and the like of the organic EL device may be improved. Specifically, the compound represented by Chemical Formula 1 may be preferably included in an organic EL device as a green and/or red phosphorescent host, a fluorescent host, or a dopant material. In particular, the compound represented by Chemical Formula 1 of the present invention may preferably be a green phosphorescent exciplex N-type host material for a high-efficiency light emitting layer.

A structure of the organic EL device of the present invention is not particularly limited, and may be, for example, a structure in which a substrate, an anode, a hole injection layer, a hole transport layer, a light emitting auxiliary layer, a light emitting layer, an electron transport layer, and a cathode are sequentially stacked. In such a case, at least one of the hole injection layer, the hole transport layer, the light emitting auxiliary layer, the light emitting layer, the electron transport layer and the electron injection layer may include the compound represented by Chemical Formula 1, and preferably, the light emitting layer, more preferably a phosphorescent host, may include the compound represented by Chemical Formula 1. Meanwhile, an electron injection layer may be additionally stacked on the electron transport layer.

The organic EL device of the present invention may have a structure in which an insulating layer or an adhesive layer is inserted at an interface between the electrode and the organic layer.

The organic EL device of the present invention may be prepared using materials and methods known in the art to form organic layers and electrodes, except that one or more layers of the aforementioned organic layers include the compound represented by Chemical Formula 1.

The organic layer may be formed by a vacuum deposition method or a solution coating method. Examples of the solution coating method may include, but not limited to, spin coating, dip coating, doctor blading, inkjet printing, thermal transfer or the like.

The substrate used in preparation of the organic EL device of the present invention is not particularly limited, and non-limiting examples thereof may include silicon wafers, quartz, glass plates, metal plates, plastic films, sheets or the like.

In addition, an anode material may use any anode material known in the art without limitation. Examples of the anode material may include, but not limited to, a metal such as vanadium, chromium, copper, zinc, and gold or an alloy thereof; metal oxides such as zinc oxide, indium oxide, indium tin oxide (ITO), or indium zinc oxide (IZO); combination of oxide with metal such as ZnO:Al or SnO₂:Sb; conductive polymers such as polythiophene, poly(3-methylthiophene), poly [3,4-(ethylene-1,2-dioxy) thiophene](PEDT), polypyrrole or polyaniline; carbon black or the like.

In addition, a cathode material may use any cathode material known in the art without limitation. Examples of the cathode material may include, but not limited to, a metal such as magnesium, calcium, sodium, potassium, titanium, indium, yttrium, lithium, gadolinium, aluminum, silver, tin, or lead or an alloy thereof; a multi-layered material such as LiF/Al or LiO₂/Al or the like.

In addition, materials for the hole injection layer, the hole transport layer, the electron injection layer, and the electron transport layer are not particularly limited, and conventional materials known in the art may be used without limitation.

Hereinafter, the present invention will be described in detail through examples. However, the following examples are only to illustrate the present invention, and the present invention is not limited by the following examples.

### Preparation Examples 1 to 5

### [Preparation Example 1] Synthesis of 2-(4-bromopyridin-3-yl)-4,6-diphenyl-1,3,5-triazine (B01)

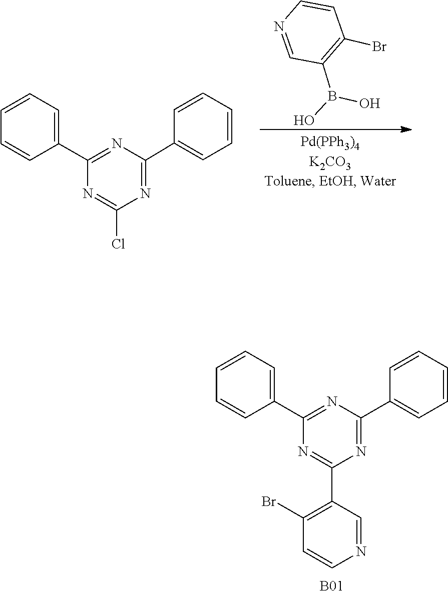

2-chloro-4,6-diphenyl-1,3,5-triazine (30.0 g, 112.1 mmol), (4-bromopyridin-3-yl) boronic acid (22.1 g, 112.1 mmol), Pd(PPh₃)₄ (3.9 g, 3.4 mmol), and K₂CO₃ (46.5 g, 336.2 mmol) were added to 300 ml of Toluene, 45 ml of Ethanol, and 45 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 2-(4-bromopyridin-3-yl)-4,6-diphenyl-1,3,5-triazine (43.6 g, 112.1 mmol, yield 81%).

Mass: [(M+H)⁺]: 391

### [Preparation Example 2] Synthesis of 2-([1,1′-biphenyl]-3-yl)-4-(5-bromopyridin-2-yl)-6-phenyl-1,3,5-triazine (B02)

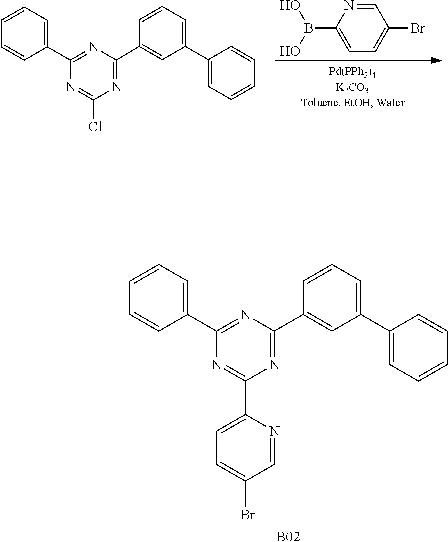

2-([1,1′-biphenyl]-3-yl)-4-chloro-6-phenyl-1,3,5-triazine (30.0 g, 87.3 mmol), (5-bromopyridin-2-yl)boronic acid (17.6 g, 87.3 mmol), Pd(PPh₃)₄ (3.0 g, 2.6 mmol), and K₂CO₃ (36.2 g, 261.8 mmol) were added to 300 ml of Toluene, 45 ml of Ethanol, and 45 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 2-([1,1′-biphenyl]-3-yl)-4-(5-bromopyridin-2-yl)-6-phenyl-1,3,5-triazine (40.6 g, 87.3 mmol, yield 75%).

Mass: [(M+H)⁺]: 466

### [Preparation Example 3] Synthesis of 2-([1,1′-biphenyl]-3-yl)-4-(4-bromopyridin-3-yl)-6-phenyl-1,3,5-triazine (B03)

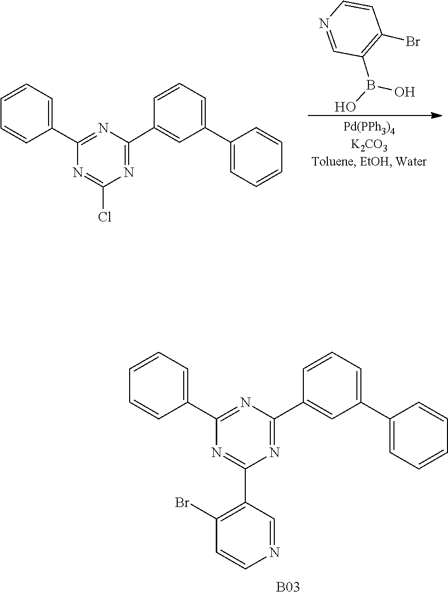

2-([1,1′-biphenyl]-3-yl)-4-chloro-6-phenyl-1,3,5-triazine (30.0 g, 87.3 mmol), (4-bromopyridin-3-yl)boronic acid (17.6 g, 87.3 mmol), Pd(PPh₃)₄ (3.0 g, 2.6 mmol), and K₂CO₃ (36.2 g, 261.8 mmol) were added to 300 ml of Toluene, 45 ml of Ethanol, and 45 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 2-([1,1′-biphenyl]-3-yl)-4-(4-bromopyridin-3-yl)-6-phenyl-1,3,5-triazine (40.6 g, 87.3 mmol, yield 72%).

Mass: [(M+H)⁺]: 466

### [Preparation Example 4] Synthesis of 2-(5-bromopyridin-2-yl)-4-(naphthalen-2-yl)-6-phenyl-1,3,5-triazine (B04)

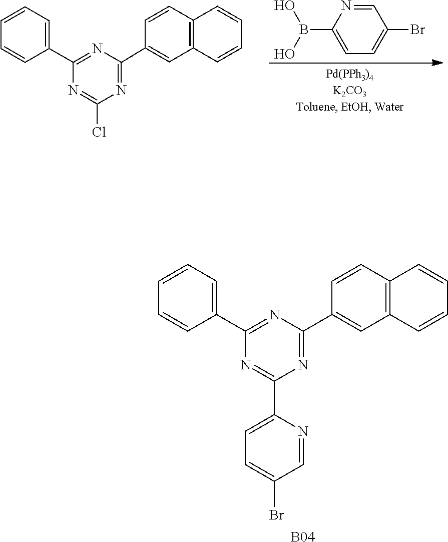

2-chloro-4-(naphthalen-2-yl)-6-phenyl-1,3,5-triazine (30.0 g, 94.4 mmol), (5-bromopyridin-2-yl)boronic acid (19.1 g, 94.4 mmol), Pd(PPh₃)₄ (3.3 g, 2.8 mmol), and K₂CO₃ (39.1 g, 283.2 mmol) were added to 300 ml of Toluene, 45 ml of Ethanol, and 45 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 2-(5-bromopyridin-2-yl)-4-(naphthalen-2-yl)-6-phenyl-1,3,5-triazine (41.5 g, 94.4 mmol, yield 78%).

Mass: [(M+H)⁺]: 441

### [Preparation Example 5] Synthesis of 2-(4-bromopyridin-3-yl)-4-(naphthalen-2-yl)-6-phenyl-1,3,5-triazine (B05)

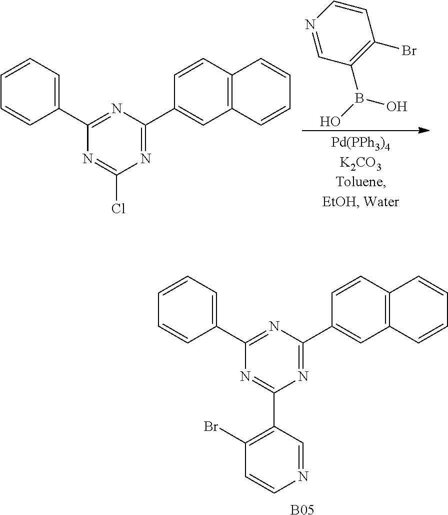

2-chloro-4-(naphthalen-2-yl)-6-phenyl-1,3,5-triazine (30.0 g, 94.4 mmol), (4-bromopyridin-3-yl)boronic acid (19.1 g, 94.4 mmol), Pd(PPh₃)₄ (3.3 g, 2.8 mmol), and K₂CO₃ (39.1 g, 283.2 mmol) were added to 300 ml of Toluene, 45 ml of Ethanol, and 45 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 2-(4-bromopyridin-3-yl)-4-(naphthalen-2-yl)-6-phenyl-1,3,5-triazine (41.5 g, 94.4 mmol, yield 74%).

Mass: [(M+H)⁺]: 441

### Synthesis Examples 1-12

### [Synthesis Example 1] Synthesis of 7′-(6-(4,6-diphenyl-1,3,5-triazin-2-yl)pyridin-3-yl)spiro[cyclohexane-1,9′-fluorene]-2′-carbonitrile (Compound 001)

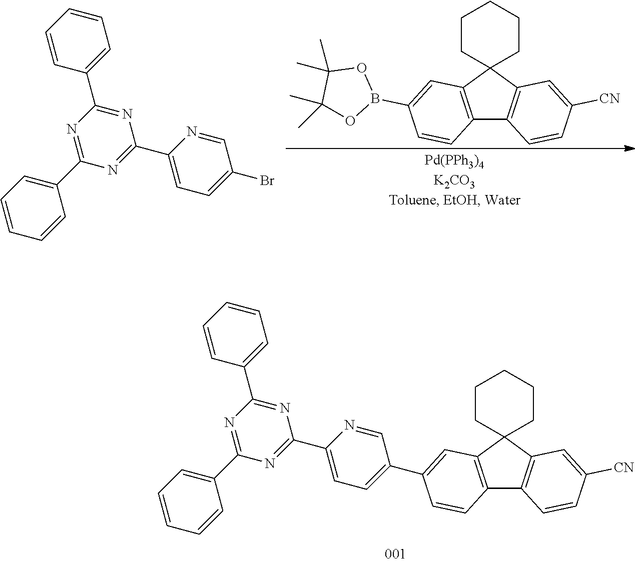 [SMILES: Brc1ccc(-c2nc(-c3ccccc3)nc(-c3ccccc3)n2)nc1 ; CC1(C)OB(c2ccc3c(c2)C2(CCCCC2)c2cc(C#N)ccc2-3)OC1(C)C ; N#Cc1ccc2c(c1)C1(CCCCC1)c1cc(-c3ccc(-c4nc(-c5ccccc5)nc(-c5ccccc5)n4)nc3)ccc1-2]

2-(5-bromopyridin-2-yl)-4,6-diphenyl-1,3,5-triazine (15.0 g, 38.5 mmol), 7′-(4,4,5,5-tetramethyl-1,3,2-dioxaborolan-2-yl)spiro[cyclohexane-1,9′-fluorene]-2′-carbonitrile (14.8 g, 38.5 mmol), Pd(PPh₃)₄ (1.3 g, 1.2 mmol), and K₂CO₃ (16.0 g, 115.6 mmol) were added to 150 ml of Toluene, 22.5 ml of Ethanol, and 22.5 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 7′-(6-(4,6-diphenyl-1,3,5-triazin-2-yl)pyridin-3-yl)spiro[cyclohexane-1,9′-fluorene]-2′-carbonitrile (21.9 g, 38.5 mmol, yield 72%).

Mass: [(M+H)⁺]:569

### [Synthesis Example 2] Synthesis of 2′-(6-(4,6-diphenyl-1,3,5-triazin-2-yl)pyridin-3-yl)spiro[cyclohexane-1,9′-fluorene]-5′-carbonitrile (Compound 003)

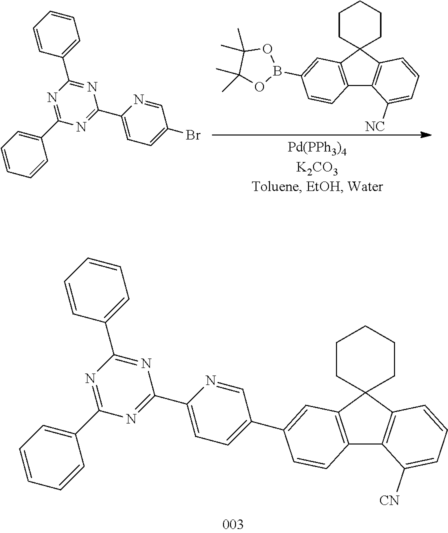

2-(5-bromopyridin-2-yl)-4,6-diphenyl-1,3,5-triazine (15.0 g, 38.5 mmol), 2′-(4,4,5,5-tetramethyl-1,3,2-dioxaborolan-2-yl)spiro[cyclohexane-1,9′-fluorene]-5′-carbonitrile (14.8 g, 38.5 mmol), Pd(PPh₃)₄ (1.3 g, 1.2 mmol), and K₂CO₃ (16.0 g, 115.6 mmol) were added to 150 ml of Toluene, 22.5 ml of Ethanol, and 22.5 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 2′-(6-(4,6-diphenyl-1,3,5-triazin-2-yl)pyridin-3-yl)spiro[cyclohexane-1,9′-fluorene]-5′-carbonitrile (21.9 g, 38.5 mmol, yield 75%).

Mass: [(M+H)⁺]:569

### [Synthesis Example 3] Synthesis of 7′-(2-(4,6-diphenyl-1,3,5-triazin-2-yl)pyridin-4-yl)spiro[cyclohexane-1,9′-fluorene]-2′-carbonitrile (Compound 004)

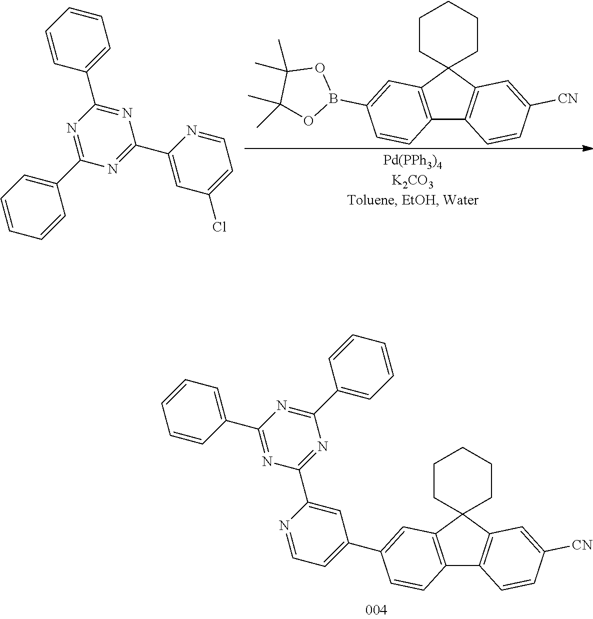

2-(4-chloropyridin-2-yl)-4,6-diphenyl-1,3,5-triazine (15.0 g, 43.5 mmol), 7′-(4,4,5,5-tetramethyl-1,3,2-dioxaborolan-2-yl)spiro[cyclohexane-1,9′-fluorene]-2′-carbonitrile (16.8 g, 43.5 mmol), Pd(PPh₃)₄ (1.5 g, 1.3 mmol), and K₂CO₃ (18.0 g, 130.5 mmol) were added to 150 ml of Toluene, 22.5 ml of Ethanol, and 22.5 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 7′-(2-(4,6-diphenyl-1,3,5-triazin-2-yl)pyridin-4-yl)spiro[cyclohexane-1,9′-fluorene]-2′-carbonitrile (24.7 g, 43.5 mmol, yield 80%).

Mass: [(M+H)⁺]:569

### [Synthesis Example 4] Synthesis of 7-(6-(4,6-diphenyl-1,3,5-triazin-2-yl)pyridin-3-yl)-9,9-dimethyl-9H-fluorene-4-carbonitrile (Compound 013)

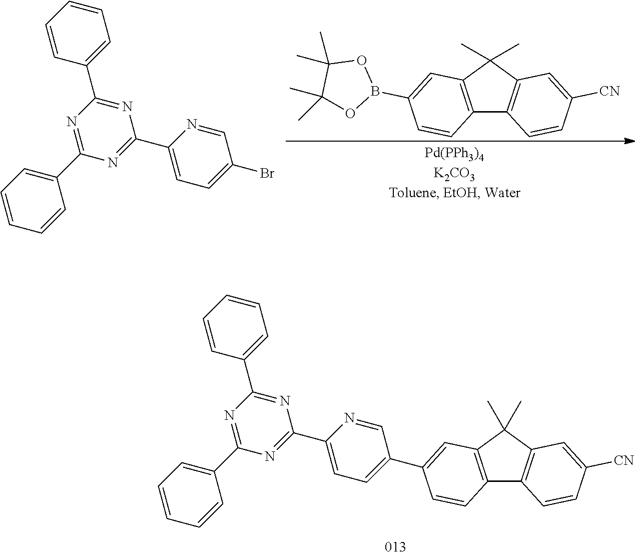 [SMILES: Brc1ccc(-c2nc(-c3ccccc3)nc(-c3ccccc3)n2)nc1 ; CC1(C)c2cc(C#N)ccc2-c2ccc(B3OC(C)(C)C(C)(C)O3)cc21 ; CC1(C)c2cc(C#N)ccc2-c2ccc(-c3ccc(-c4nc(-c5ccccc5)nc(-c5ccccc5)n4)nc3)cc21]

2-(5-bromopyridin-2-yl)-4,6-diphenyl-1,3,5-triazine (15.0 g, 38.5 mmol), 9,9-dimethyl-7-(4,4,5,5-tetramethyl-1,3,2-dioxaborolan-2-yl)-9H-fluorene-2-carbonitrile (13.3 g, 38.5 mmol), Pd(PPh₃)₄ (1.3 g, 1.2 mmol), and K₂CO₃ (16.0 g, 115.6 mmol) were added to 150 ml of Toluene, 22.5 ml of Ethanol, and 22.5 ml of Water, and the resulting mixture was heated to reflux for 2 hours.

After the reaction was completed, the reaction mixture was extracted with Methylene chloride, and the extracted organic layer was concentrated after removing moisture using Magnesium Sulfate, and purified by column chromatography to obtain 7-(6-(4,6-diphenyl-1,3,5-triazin-2-yl)pyridin-3-yl)-9,9-dimethyl-9H-fluorene-4-carbonitrile (20.3 g, 38.5 mmol, yield 79%).

Mass: [(M+H)⁺]:528

### [Synthesis Example 5] Synthesis of 7-(2-(4,6-diphenyl-1,3,5-triazin-2-yl)pyridin-4-yl)-9,9-dimethyl-9H-fluorene-2-carbonitrile (Compound 015)

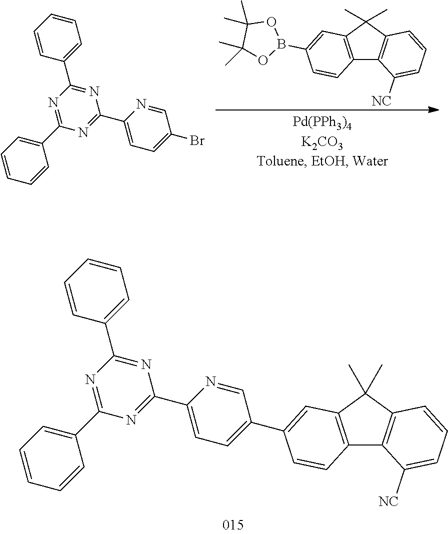 [SMILES: Brc1ccc(-c2nc(-c3ccccc3)nc(-c3ccccc3)n2)nc1 ; CC1(C)c2cc(B3OC(C)(C)C(C)(C)O3)ccc2-c2c(C#N)cccc21 ; CC1(C)c2cc(-c3ccc(-c4nc(-c5ccccc5)nc(-c5ccccc5)n4)nc3)ccc2-c2c(C#N)cccc21]

2-(5-bromopyridin-2-yl)-4,6-diphenyl-1,3,5-triazine (15.0 g, 38.5 mmol), 9,9-dimethyl-7-(4,4,5,5-tetramethyl-1,3,2-dioxaborolan-2-yl)-9H-fluorene-4-carbonitrile (13.3 g, 38.5 mmol), Pd(PPh₃)₄ (1.3 g, 1.2 mmol), and K₂CO₃ (16.0 g, 115.6 mmol) were added to 150 ml of Toluene, 22.5 ml of Ethanol, and 22.5 ml of Water, and the resulting mixture was heated to reflux for 2 hours.

After the reaction was completed, the reaction mixture was extracted with Methylene chloride, and the extracted organic layer was concentrated after removing moisture using Magnesium Sulfate, and purified by column chromatography to obtain 7-(2-(4,6-diphenyl-1,3,5-triazin-2-yl)pyridin-4-yl)-9,9-dimethyl-9H-fluorene-2-carbonitrile (20.3 g, 38.5 mmol, yield 72%).

Mass: [(M+H)⁺]:529

### [Synthesis Example 6] Synthesis of 7-(2-(4,6-diphenyl-1,3,5-triazin-2-yl)pyridin-4-yl)-9,9-dimethyl-9H-fluorene-2-carbonitrile (Compound 016)

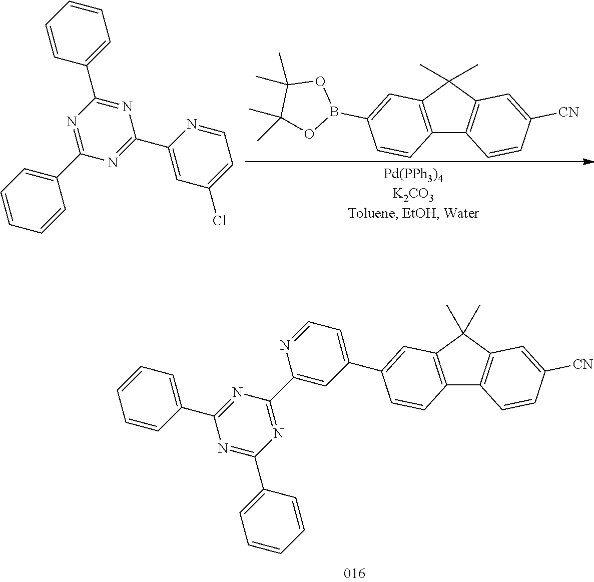

2-(4-chloropyridin-2-yl)-4,6-diphenyl-1,3,5-triazine (15.0 g, 43.5 mmol), 9,9-dimethyl-7-(4,4,5,5-tetramethyl-1,3,2-dioxaborolan-2-yl)-9H-fluorene-2-carbonitrile (15.0 g, 43.5 mmol) Pd(PPh₃)₄ (1.5 g, 1.3 mmol), and K₂CO₃ (18.0 g, 130.5 mmol) were added to 150 ml of Toluene, 22.5 ml of Ethanol, and 22.5 ml of Water, and the resulting mixture was heated to reflux for 2 hours.

After the reaction was completed, the reaction mixture was extracted with Methylene chloride, and the extracted organic layer was concentrated after removing moisture using Magnesium Sulfate, and purified by column chromatography to obtain 7-(2-(4,6-diphenyl-1,3,5-triazin-2-yl)pyridin-4-yl)-9,9-dimethyl-9H-fluorene-2-carbonitrile (23.0 g, 43.5 mmol, yield 75%).

Mass: [(M+H)⁺]:529

### [Synthesis Example 7] Synthesis of 7′-(3-(4,6-diphenyl-1,3,5-triazin-2-yl)pyridin-4-yl)spiro[cyclohexane-1,9′-fluorene]-2′-carbonitrile (Compound 022)

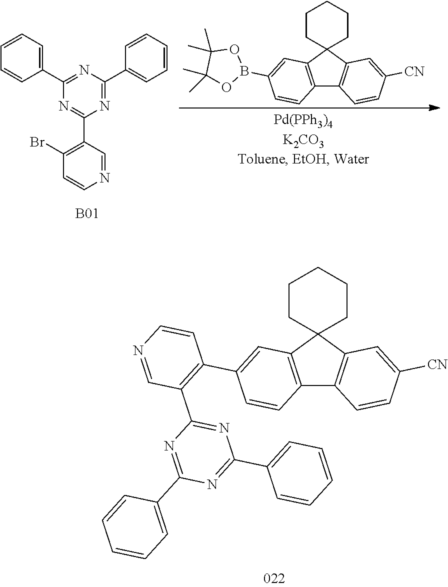

B01 (15.0 g, 38.5 mmol) synthesized by the method of Preparation Example 1, 2-(4-bromopyridin-3-yl)-4,6-diphenyl-1,3,5-triazine (14.8 g, 38.5 mmol), Pd(PPh₃)₄ (1.3 g, 1.2 mmol), and K₂CO₃ (16.0 g, 115.6 mmol) were added to 150 ml of Toluene 150 ml, 22.5 ml of Ethanol, and 22.5 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 7′-(3-(4,6-diphenyl-1,3,5-triazin-2-yl)pyridin-4-yl)spiro[cyclohexane-1,9′-fluorene]-2′-carbonitrile (21.9 g, 38.5 mmol, yield 77%).

Mass: [(M+H)⁺]:569

### [Synthesis Example 8] Synthesis of 5-(3-(4,6-diphenyl-1,3,5-triazin-2-yl)pyridin-4-yl)-9,9-dimethyl-9H-fluorene-2-carbonitrile (Compound 040)

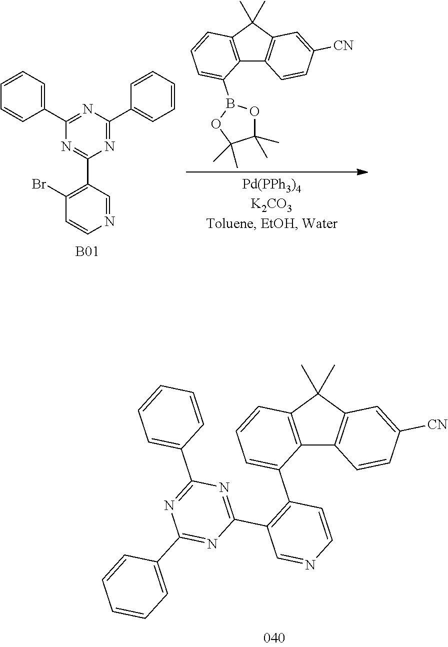

B01 (15.0 g, 38.5 mmol) synthesized by the method of Preparation Example 1, 9,9-dimethyl-5-(4,4,5,5-tetramethyl-1,3,2-dioxaborolan-2-yl)-9H-fluorene-2-carbonitrile (13.3 g, 38.5 mmol), Pd(PPh₃)₄ (1.3 g, 1.2 mmol), and K₂CO₃ (16.0 g, 115.6 mmol) were added to 150 ml of Toluene, 22.5 ml of Ethanol, and 22.5 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 5-(3-(4,6-diphenyl-1,3,5-triazin-2-yl)pyridin-4-yl)-9,9-dimethyl-9H-fluorene-2-carbonitrile (20.3 g, 38.5 mmol, yield 80%).

Mass: [(M+H)⁺]:528

### [Synthesis Example 9] Synthesis of 2′-(6-(4-([1,1′-biphenyl]-3-yl)-6-phenyl-1,3,5-triazin-2-yl)pyridin-3-yl)spiro[cyclohexane-1,9′-fluorene]-7′-carbonitrile (Compound 046)

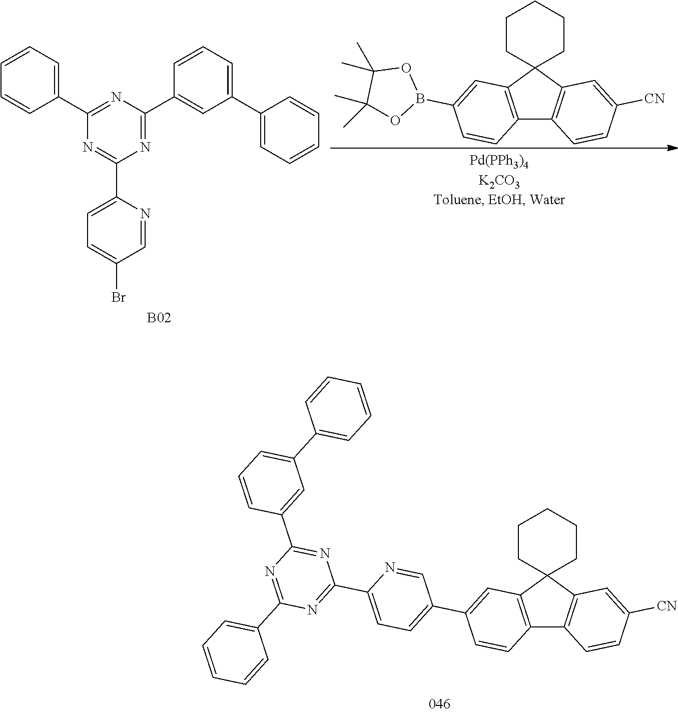

B02 (15.0 g, 32.2 mmol) synthesized by the method of Preparation Example 2, 7′-(4,4,5,5-tetramethyl-1,3,2-dioxaborolan-2-yl)spiro[cyclohexane-1,9′-fluorene]-2′-carbonitrile (12.4 g, 32.2 mmol), Pd(PPh₃)₄ (1.1 g, 1.00 mmol), and K₂CO₃ (13.4 g, 96.7 mmol) were added to 150 ml of Toluene, 22.5 ml of Ethanol, and 22.5 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 2′-(6-(4-([1,1′-biphenyl]-3-yl)-6-phenyl-1,3,5-triazin-2-yl)pyridin-3-yl)spiro[cyclohexane-1,9′-fluorene]-7′-carbonitrile (20.8 g, 32.2 mmol, yield 76%).

Mass: [(M+H)⁺]:644

### [Synthesis Example 10] Synthesis of 2′-(3-(4-([1,1′-biphenyl]-3-yl)-6-phenyl-1,3,5-triazin-2-yl)pyridin-4-yl)spiro[cyclohexane-1,9′-fluorene]-7′-carbonitrile (Compound 067)

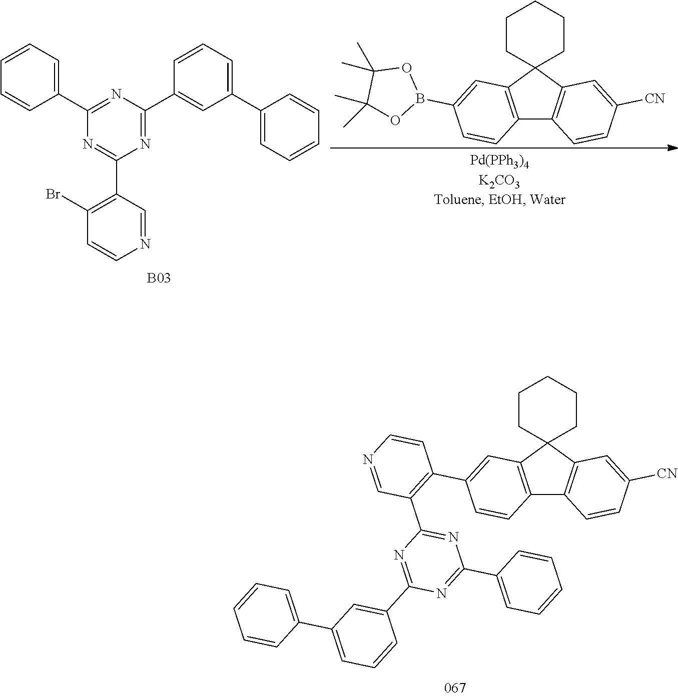

B03 (15.0 g, 32.2 mmol) synthesized by the method of Preparation Example 3, 7′-(4,4,5,5-tetramethyl-1,3,2-dioxaborolan-2-yl)spiro[cyclohexane-1,9′-fluorene]-2′-carbonitrile (12.4 g, 32.2 mmol), Pd(PPh₃)₄ (1.1 g, 1.00 mmol), and K₂CO₃ (13.4 g, 96.7 mmol) were added to 150 ml of Toluene, 22.5 ml of Ethanol, and 22.5 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 2′-(3-(4-([1,1′-biphenyl]-3-yl)-6-phenyl-1,3,5-triazin-2-yl)pyridin-4-yl)spiro[cyclohexane-1,9′-fluorene]-7′-carbonitrile (20.8 g, 32.2 mmol, yield 80%).

Mass: [(M+H)⁺]:644

### [Synthesis Example 11] Synthesis of 7′-(6-(4-(naphthalen-2-yl)-6-phenyl-1,3,5-triazin-2-yl)pyridin-3-yl)spiro[cyclohexane-1,9′-fluorene]-2′-carbonitrile (Compound 091)

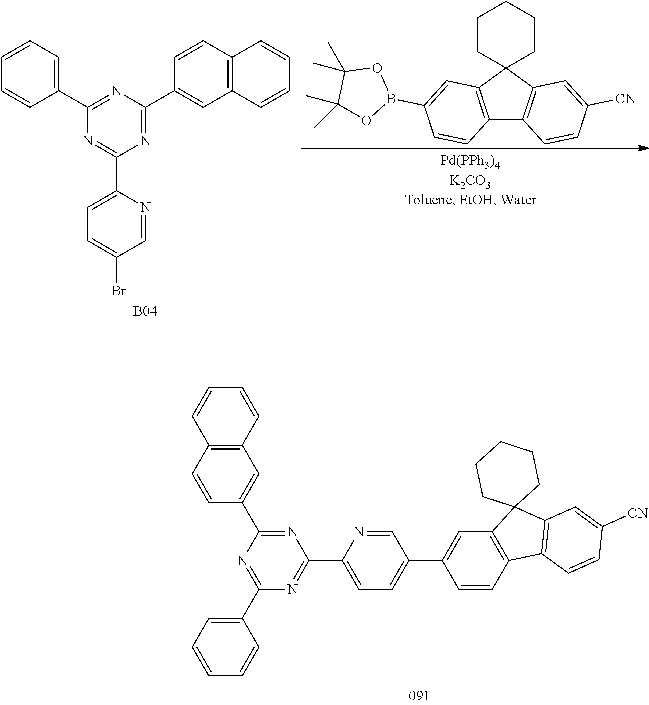

B04 (15.0 g, 34.1 mmol) synthesized by the method of Preparation Example 4, 7′-(4,4,5,5-tetramethyl-1,3,2-dioxaborolan-2-yl)spiro[cyclohexane-1,9′-fluorene]-2′-carbonitrile (13.2 g, 34.1 mmol), Pd(PPh₃)₄ (1.2 g, 1.00 mmol), and K₂CO₃ (14.2 g, 102.4 mmol) were added to 150 ml of Toluene, 22.5 ml of Ethanol, and 22.5 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 7′-(6-(4-(naphthalen-2-yl)-6-phenyl-1,3,5-triazin-2-yl)pyridin-3-yl)spiro[cyclohexane-1,9′-fluorene]-2′-carbonitrile (21.1 g, 34.1 mmol, yield 82%).

Mass: [(M+H)⁺]:619

### [Synthesis Example 12] Synthesis of 7′-(3-(4-(naphthalen-2-yl)-6-phenyl-1,3,5-triazin-2-yl)pyridin-4-yl)spiro[cyclohexane-1,9′-fluorene]-2′-carbonitrile (Compound 112)

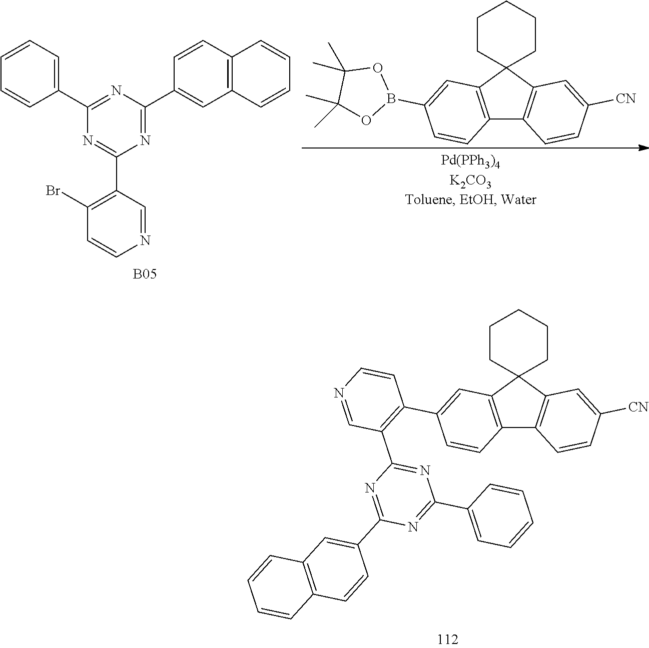

B05 (15.0 g, 34.1 mmol) synthesized by the method of Preparation Example 5, 7′-(4,4,5,5-tetramethyl-1,3,2-dioxaborolan-2-yl)spiro[cyclohexane-1,9′-fluorene]-2′-carbonitrile (13.2 g, 34.1 mmol), Pd(PPh₃)₄ (1.2 g, 1.00 mmol), and K₂CO₃ (14.2 g, 102.4 mmol) were added to 150 ml of Toluene, 22.5 ml of Ethanol, and 22.5 ml of Water, and the resulting mixture was heated to reflux for 2 hours. After the reaction was completed, the reaction mixture was extracted with Methylene chloride, and the extracted organic layer was concentrated after removing moisture using Magnesium Sulfate, and purified by column chromatography to obtain 7′-(3-(4-(naphthalen-2-yl)-6-phenyl-1,3,5-triazin-2-yl)pyridin-4-yl)spiro[cyclohexane-1,9′-fluorene]-2′-carbonitrile (21.1 g, 34.1 mmol, yield 81%).

Mass: [(M+H)⁺]:619

### [Example 1] Manufacturing of Blue Organic EL Device

The compound 001 synthesized in the above synthesis example 1 wase subjected to high-purity sublimation purification in a conventionally known method, and then blue organic EL device was manufactured according to the following procedure.

A glass substrate thin-film-coated with indium tin oxide (ITO) to a thickness of 1,200 Å was washed with distilled water ultrasonically. After washing with distilled water was completed, the glass substrate was ultrasonically washed with a solvent, such as isopropyl alcohol, acetone and methanol, dried, transferred to a UV OZONE cleaner (Power sonic 405, Hwasin Tech), cleaned for 5 minutes using UV, and then transferred to a vacuum evaporator.

On the prepared ITO transparent electrode, Compound A and Compound B were co-deposited at a weight ratio of 98:2 to from a hole injection layer with a thickness of 100 Å, and then Compound A was deposited on the hole injection layer to form a hole transport layer with a thickness of 1400 Å. Thereafter, Compound C was deposited with a thickness of 50 Å on the hole transport layer to form a hole transport auxiliary layer, and Compound D and Compound E were co-deposited at a weight ratio of 98:2 to form an emissive layer with a thickness of 200 Å. Thereafter, Compound F was deposited on the emissive layer to form an auxiliary electron transport layer with a thickness of 50 Å, and then Compound 001 and Compound G were co-deposited at a weight ratio of 1:1 to form an electron transport layer with a thickness of 300 Å. Thereafter, LiF was deposited on the electron transport layer to form an electron injection layer with a thickness of 10 Å, and then Al was deposited on the electron injection layer to form a cathode with a thickness of 1000 Å, thereby fabricating an organic electroluminescent device.

In such a case, the structures of Compounds A to G are each as follows.

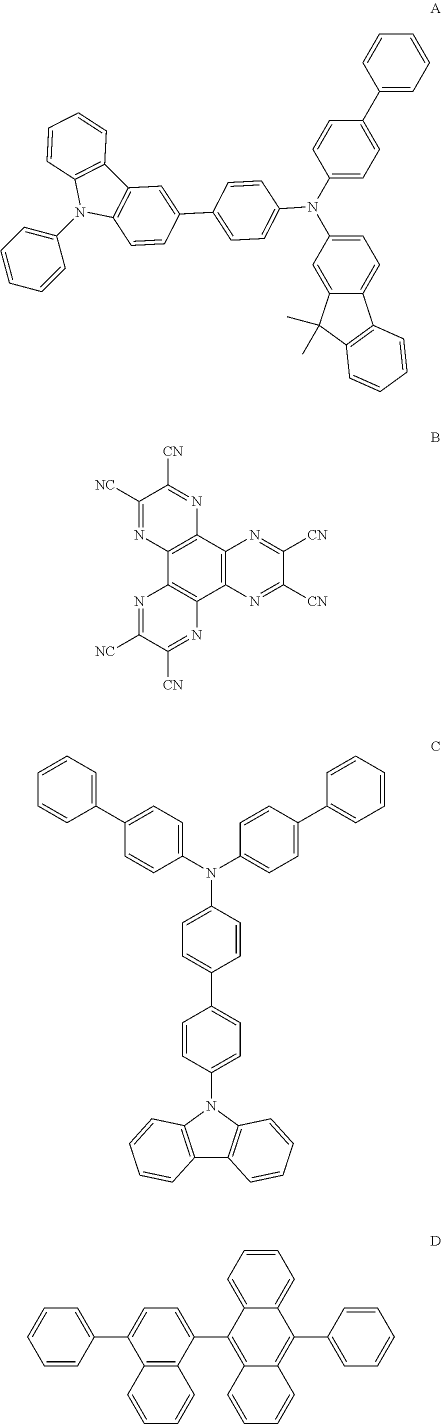

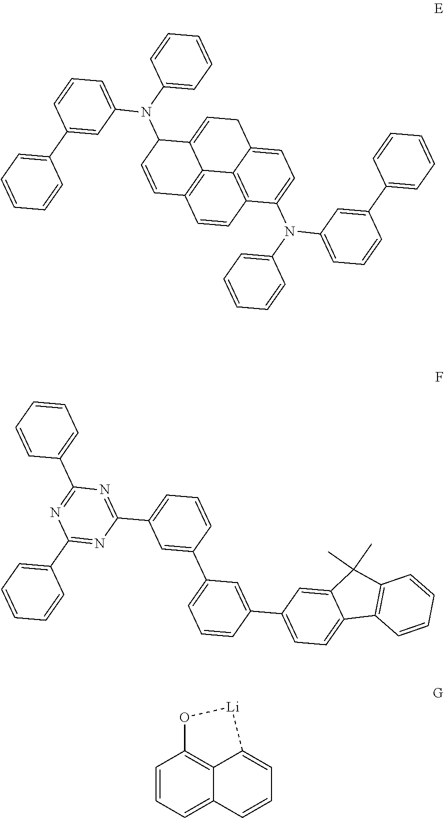

### [Examples 2 to 12] Manufacturing of Blue Organic EL Device

A blue organic EL device of Examples 2 to 12 were manufactured in the same manner as in Example 1, except that Compounds as shown in Table 1 were used instead of Compound 001 used as the electron transport layer material in Example 1.

### [Comparative Examples 1 to 5] Manufacturing of Blue Organic EL Device

A blue organic EL device of Comparative Examples 1 to 5 were manufactured in the same manner as in Example 1, except that Compounds H to L were used instead of Compound 001 used as the electron transport layer material in Example 1.

In such a case, the structures of Compounds H to L used in Comparative Examples 1 to 5 are each as follows.

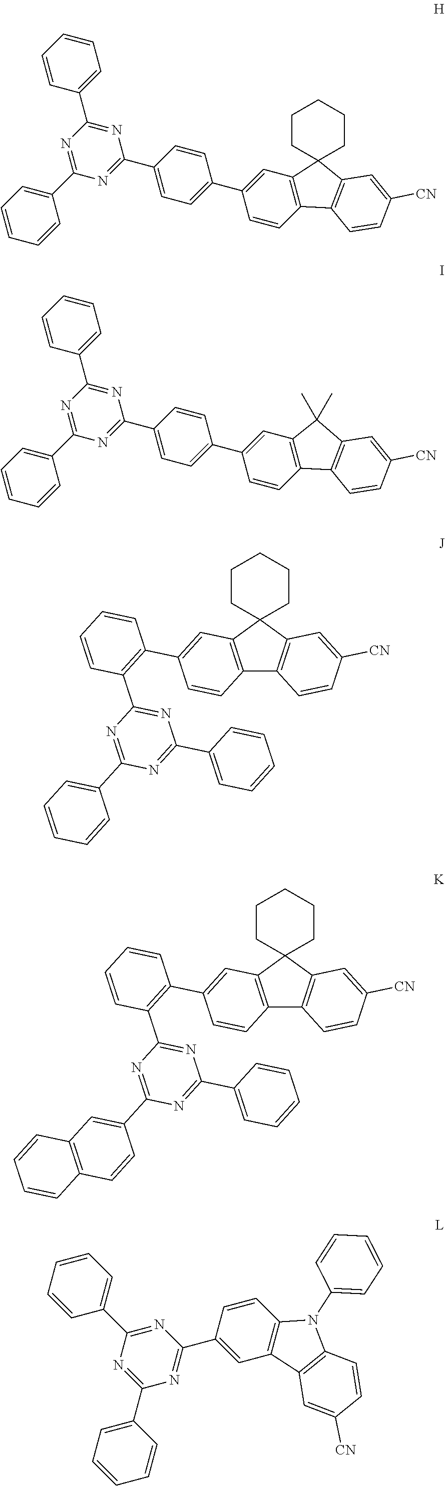 [SMILES: N#Cc1ccc2c(c1)C1(CCCCC1)c1cc(-c3ccc(-c4nc(-c5ccccc5)nc(-c5ccccc5)n4)cc3)ccc1-2 ; CC1(C)c2cc(C#N)ccc2-c2ccc(-c3ccc(-c4nc(-c5ccccc5)nc(-c5ccccc5)n4)cc3)cc21 ; N#Cc1ccc2c(c1)C1(CCCCC1)c1cc(-c3ccccc3-c3nc(-c4ccccc4)nc(-c4ccccc4)n3)ccc1-2 ; N#Cc1ccc2c(c1)C1(CCCCC1)c1cc(-c3ccccc3-c3nc(-c4ccccc4)nc(-c4ccc5ccccc5c4)n3)ccc1-2 ; N#Cc1ccc2c(c1)c1cc(-c3nc(-c4ccccc4)nc(-c4ccccc4)n3)ccc1n2-c1ccccc1]

### Evaluation Example 1

For each of the blue organic EL devices manufactured in Examples (Ex.) 1 to 12 and Comparative Examples (Comp. Ex.) 1 to 5, a driving voltage, a current efficiency at a current density of 10 mA/cm², and an emission peak were measured, and the results are shown in Table 1 below.

TABLE 1

| | Electron | Driving | | Current |
|---|---|---|---|---|
| | transport | voltage | EL peak | efficiency |
| Sample | layer | (V) | (nm) | (cd/A) |
| Ex. 1 | Compound 001 | 3.5 | 453 | 9.2 |
| Ex. 2 | Compound 003 | 3.5 | 454 | 8.8 |
| Ex. 3 | Compound 004 | 3.6 | 453 | 8.8 |
| Ex. 4 | Compound 013 | 3.7 | 452 | 8.9 |
| Ex. 5 | Compound 015 | 3.7 | 451 | 8.9 |
| Ex. 6 | Compound 016 | 3.8 | 454 | 8.5 |
| Ex. 7 | Compound 022 | 3.4 | 453 | 9.5 |
| Ex. 8 | Compound 040 | 3.4 | 455 | 9.3 |
| Ex. 9 | Compound 046 | 4.0 | 452 | 8.7 |
| Ex. 10 | Compound 067 | 3.9 | 453 | 8.8 |
| Ex. 11 | Compound 091 | 3.5 | 456 | 9.6 |
| Ex. 12 | Compound 112 | 3.4 | 453 | 9.7 |
| Comp. Ex. | Compound H | 4.9 | 455 | 7.6 |
| 1 | | | | |
| Comp. Ex. | Compound I | 5.0 | 455 | 7.5 |
| 2 | | | | |
| Comp. Ex. | Compound J | 4.9 | 456 | 7.4 |
| 3 | | | | |
| Comp. Ex. | Compound K | 4.8 | 455 | 8.0 |
| 4 | | | | |
| Comp. Ex. | Compound L | 5.5 | 453 | 6.2 |
| 5 | | | | |

As shown in Table 1, the blue organic electroluminescent devices of Examples 1 to 12 using the compounds of the present invention as electron transport layer materials exhibited excellent performance in terms of the driving voltage, light emission peak, and current efficiency, compared with the blue organic electroluminescent devices of Comparative Examples 1 to 4 using Compounds H to K as electron transport layer materials.

Specifically, the blue organic electroluminescent devices of Comparative Examples 1 to 3 using, as electron transport layer materials, compounds containing a phenylene linker between triazine and fluorene-based moieties (e.g., Compounds H to K), and the blue organic electroluminescent device of Comparative Example 5 using, as an electron transport layer material, a compound containing no linker by direct linkage of triazine and cyanocarbazole-based moieties (e.g., Compound L) exhibited poor performance in terms of the driving voltage, light emission peak, and current efficiency of devices.

However, the blue organic electroluminescent devices of Examples 1 to 12 contain, as an electron transport layer material, a compound containing a triazine moiety and a cyano fluorene-based moiety with excellent electron transport capability and stability, the moieties being linked through an azine-based linker (e.g., pyridine) that improves the electron flow, and thus can ensure low driving voltage, excellent light emission peak, and high current efficiency characteristics, compared with the devices of Comparative Examples 1 to 5.

1-14. (canceled)

15. A compound represented by Chemical Formula 1: 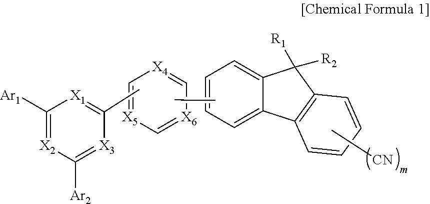
wherein in Chemical Formula 1,
X₁ to X₆ are the same as or different from each other and are each independently C(R₃) or N, provided that at least one of X₁ to X₃ and at least one of X₄ to X₆ are N;
Ar₁ and Ar₂ are the same as or different from each other and are each independently selected from the group consisting of a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ arylphosphine group, and a C₆-C₆₀ arylphosphine oxide group;
R₁ and R₂ are the same as or different from each other and are each independently a C₁-C₄₀ alkyl group, or an aliphatic hydrocarbon ring formed by bonding with any adjacent substituents;
R₃ is selected from the group consisting of a hydrogen, a deuterium (D), a halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ aryl phosphine group, a C₆-C₆₀ arylphosphine oxide group, and a C₆-C₆₀ arylamine group, or a plurality of R₃ are the same as or different from each other;
m is an integer of 1 or 2; and
the alkyl group, alkenyl group, alkynyl group, aryl group, heteroaryl group, aryloxy group, alkyloxy group, cycloalkyl group, heterocycloalkyl group, alkylsilyl group, arylsilyl group, alkylboron group, arylboron group, arylphosphine group, arylphosphine oxide group, and arylamine group of Ar₁ to Ar₂, and R₁ to R₃ may be each independently substituted with at least one substituent selected from the group consisting of a deuterium (D), a halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 ring atoms, a C₆ to Coo aryl group, a heteroaryl group having 5 to 60 ring atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to Coo arylphosphine oxide group, and a C₆ to Coo arylamine group, and wherein when the substituent is present in a plural number, they may be the same or different from each other.

16. The compound of claim 15, wherein the X₁ to X₃ containing ring is selected from the group of substituents represented by the following structural formulas: 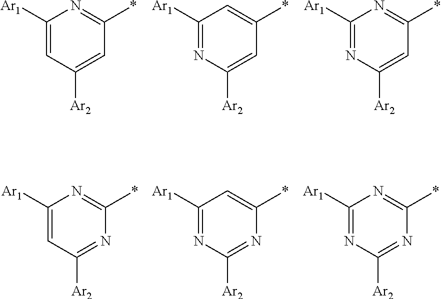
wherein in the formulas,
* indicates a site where a bond with Chemical Formula 1 is made, Ar₁ and Ar₂ are the same as defined in claim 15.

17. The compound of claim 15, wherein Ar₁ and Ar₂ are the same as or different from each other, and are each independently selected from the group consisting of a C₆ to C₆₀ aryl group and a heteroaryl group having 5 to 60 nuclear atoms.

18. The compound of claim 15, wherein Ar₁ and Ar₂ are the same as or different from each other, and are each independently selected from the following structural formulas: 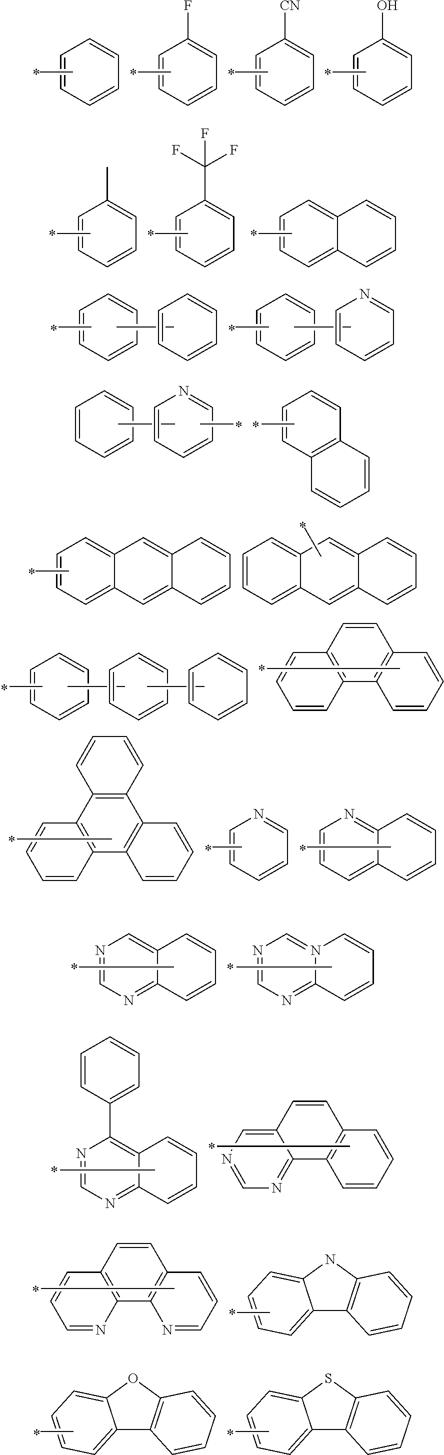 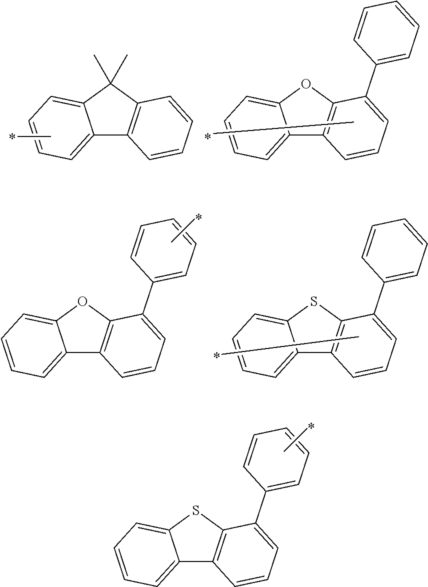
wherein in the formulas,
* indicates a site where a bond with Chemical Formula 1 is made.

19. The compound of claim 15, wherein the X₄ to X₆ containing ring is selected from the group of substituents represented by the following structural formulas: 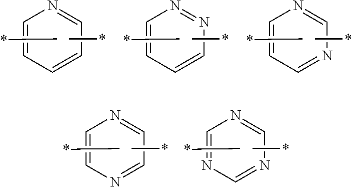
wherein in the formulas,
* indicates a site where a bond with Chemical Formula 1 is made.

20. The compound of claim 15, wherein the compound represented by Chemical Formula 1 is a compound represented by any one of Chemical Formula 2 to Chemical Formula 3 below: 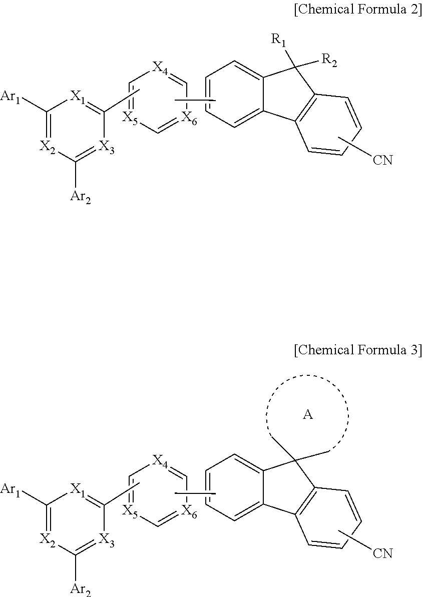
wherein in the formulas,
Ring A is an aliphatic hydrocarbon ring group having 5 to 10 carbon atoms, and
X₁ to X₆, Ar₁ to Ar₂, and R₁ to R₂ are the same as defined in claim 15.

21. The compound of claim 20, wherein the compound represented by Chemical Formula 3 is a compound represented by any one of Chemical Formula 3a to Chemical Formula 3d below: 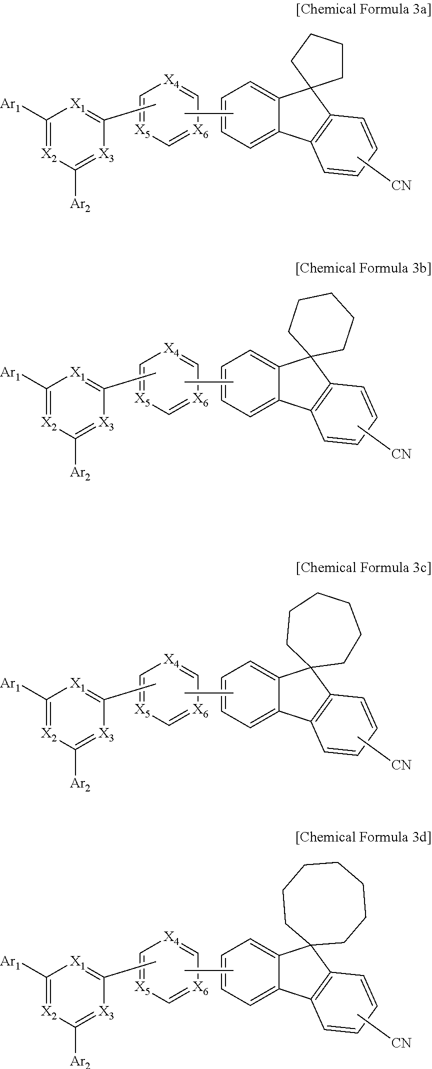
wherein in the formulas 3a to 3d,
X₁ to X₆ are the same as or different from each other and are each independently C(R₃) or N, provided that at least one of X₁ to X₃ and at least one of X₄ to X₆ are N;
Ar₁ and Ar₂ are the same as or different from each other and are each independently selected from the group consisting of a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-Coo aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ arylphosphine group, and a C₆-C₆₀ arylphosphine oxide group; and
the alkyl group, alkenyl group, alkynyl group, aryl group, heteroaryl group, aryloxy group, alkyloxy group, cycloalkyl group, heterocycloalkyl group, alkylsilyl group, arylsilyl group, alkylboron group, arylboron group, arylphosphine group, arylphosphine oxide group, and arylamine group of Ar₁ to Ar₂ may be each independently substituted with at least one substituent selected from the group consisting of a deuterium (D), a halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 ring atoms, a C₆ to Coo aryl group, a heteroaryl group having 5 to 60 ring atoms, a C₁ to C₄₀ alkyloxy group, a C₆ to Coo aryloxy group, a C₁ to C₄₀ alkylsilyl group, a C₆ to C₆₀ arylsilyl group, a C₁ to C₄₀ alkylboron group, a C₆ to Coo arylboron group, a C₆ to Coo arylphosphine group, a C₆ to Coo arylphosphine oxide group, and a C₆ to Coo arylamine group, and wherein when the substituent is present in a plural number, they may be the same or different from each other.

22. The compound of claim 15, wherein the compound represented by Chemical Formula 1 is a compound represented by any one of Chemical Formula 4 to Chemical Formula 7 below: 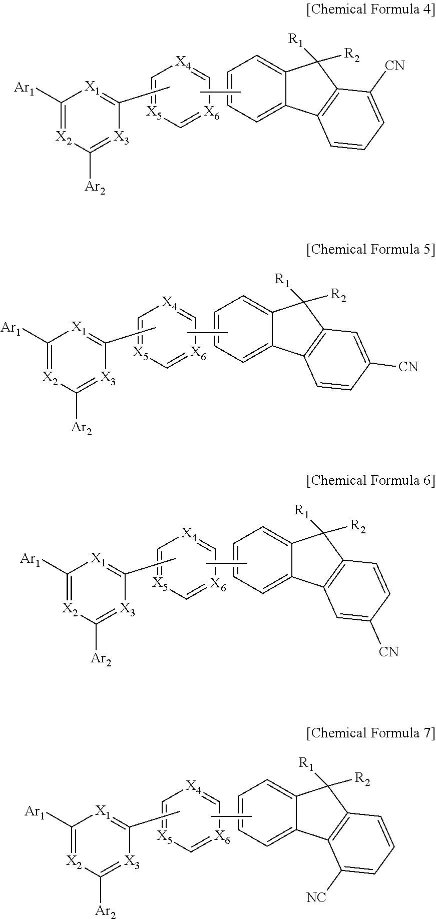
wherein in the formulas 4 to 7,
X₁ to X₆, Ar₁ to Ar₂, and R₁ to R₂ are the same as defined in claim 15.

23. The compound of claim 15, wherein the compound represented by Chemical Formula 1 is a compound represented by any one of Chemical Formula 8 to Chemical Formula 11 below: 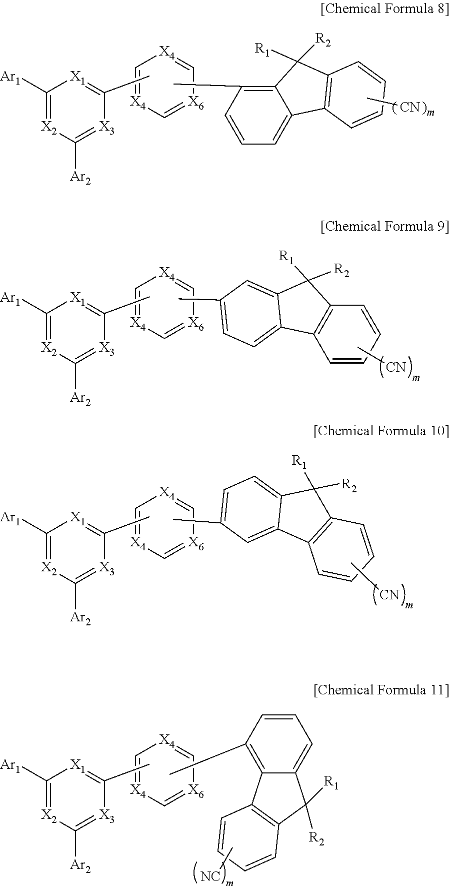
wherein in the formulas 4 to 7,
X₁ to X₆, Ar₁ to Ar₂, R₁ to R₂ and m are the same as defined in claim 15.

24. The compound of claim 15, wherein the compound represented by Chemical Formula 1 is a compound represented by any one of Chemical Formula 12 to Chemical Formula 14 below: 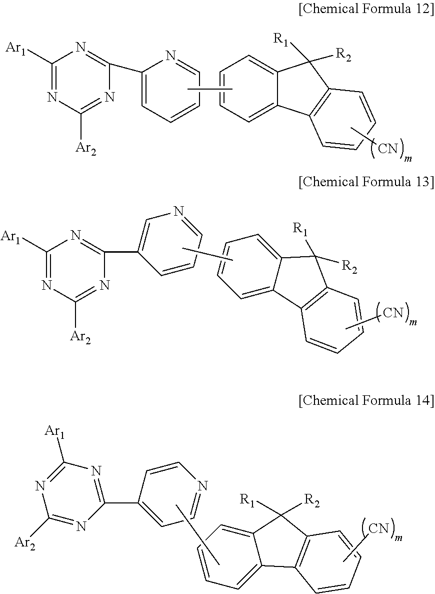
wherein in the formulas 12 to 14,
Ar₁ to Ar₂, R₁ to R₂ and m are the same as defined in claim 15.

25. The compound of claim 15, wherein the compound represented by Chemical Formula 1 is selected from compounds represented by any one of the following Chemical Formulas 1 to 135:

[chemical structure]

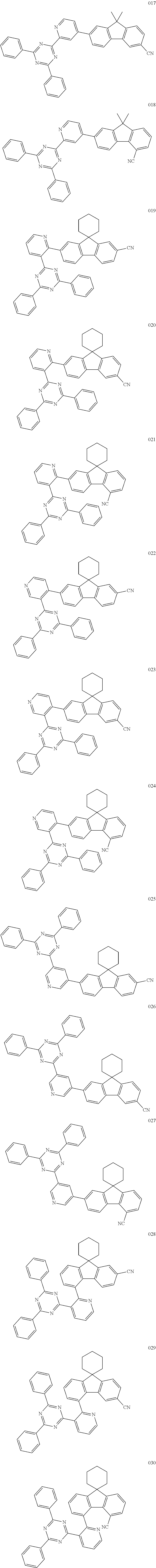

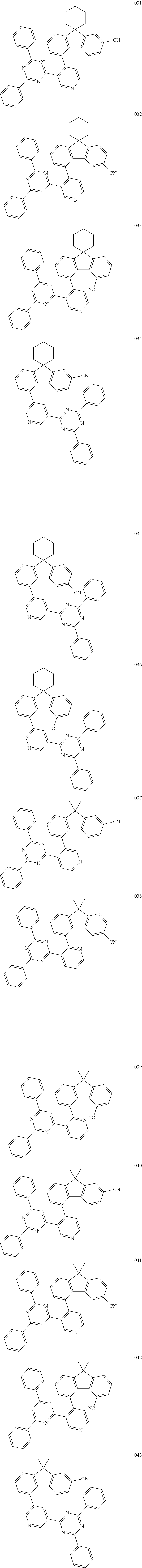

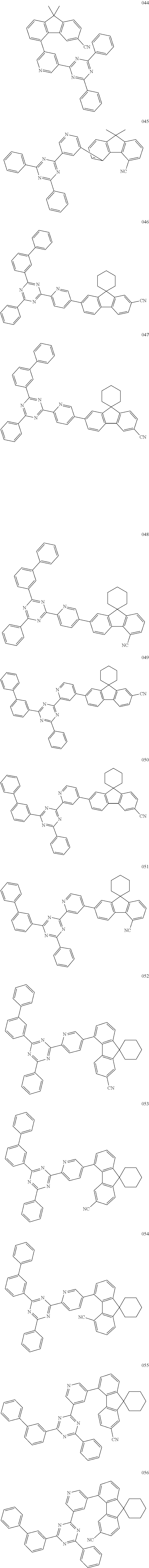

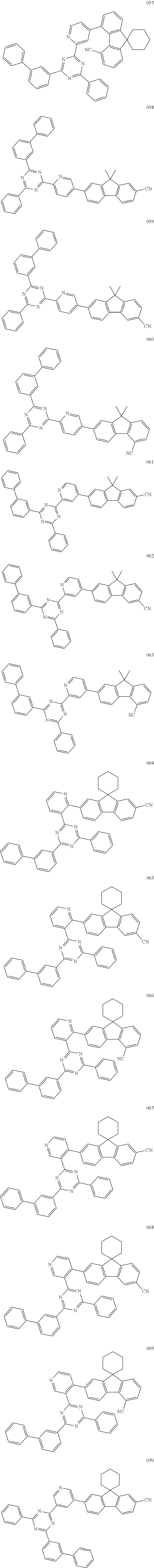

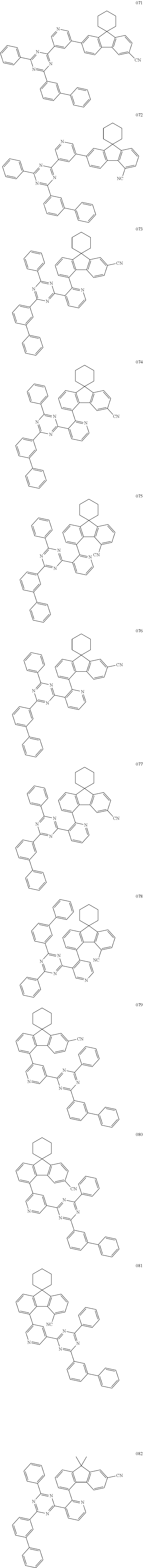

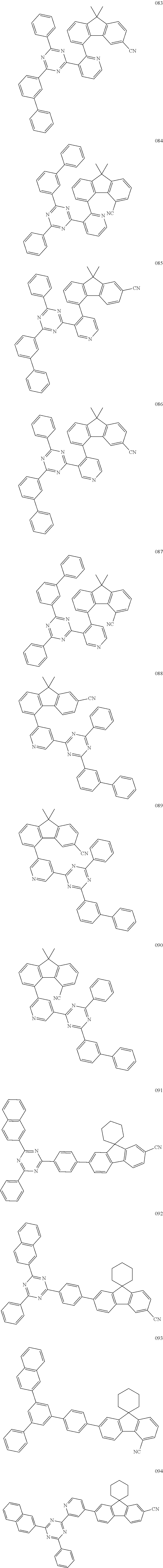

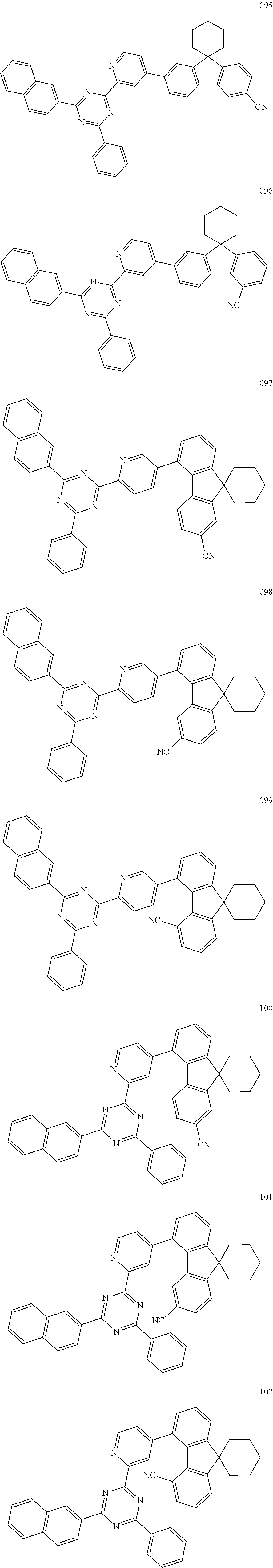

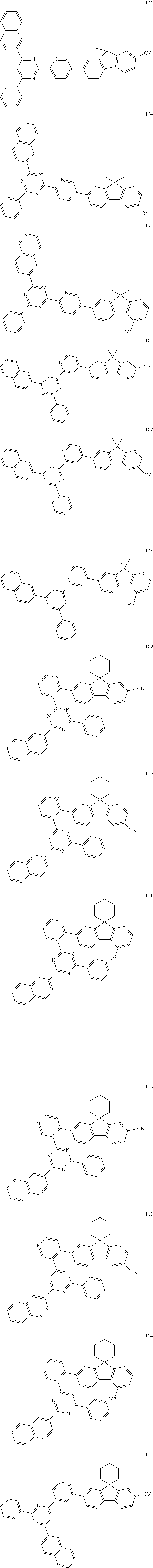

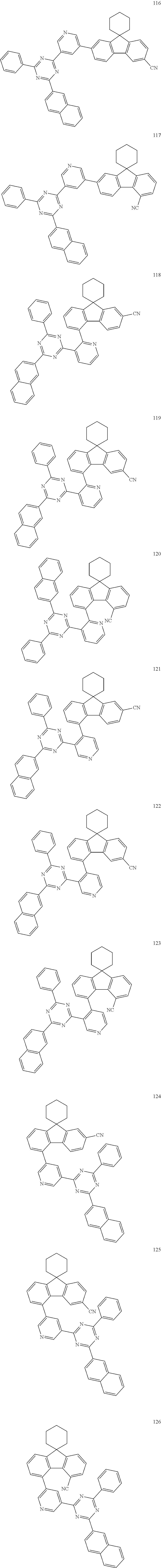

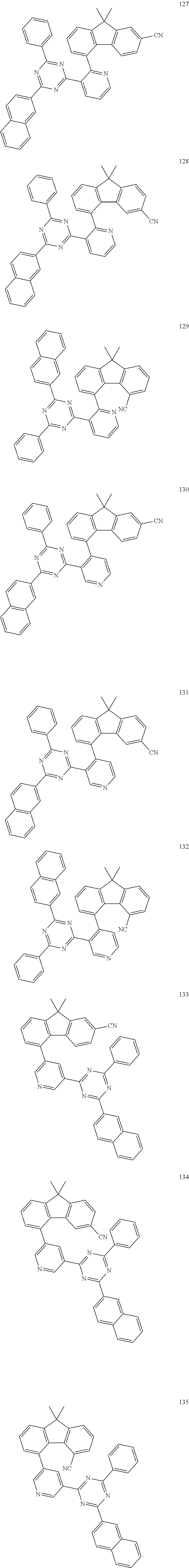

26. The compound of claim 15, wherein the compound represented by Chemical Formula 1 is used as a material of a light-emitting layer, an electron transport layer, or an auxiliary electron transport layer.

27. An organic electroluminescent device comprising:
an anode;
a cathode; and
one or more organic layers disposed between the anode and the cathode,
wherein at least one of the one or more organic layers comprises a compound represented by Chemical Formula 1: 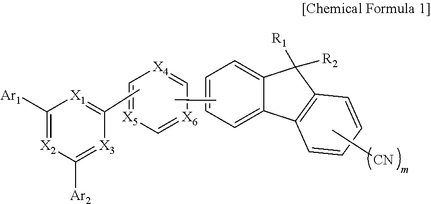
wherein in Chemical Formula 1,
X₁ to X₆ are the same as or different from each other and are each independently C(R₃) or N, provided that at least one of X₁ to X₃ and at least one of X₄ to X₆ are N;
Ar₁ and Ar₂ are the same as or different from each other and are each independently selected from the group consisting of a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ arylphosphine group, and a C₆-C₆₀ arylphosphine oxide group;
R₁ and R₂ are the same as or different from each other and are each independently a C₁-C₄₀ alkyl group, or an aliphatic hydrocarbon ring formed by bonding with any adjacent substituents;
R₃ is selected from the group consisting of a hydrogen, a deuterium (D), a halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ aryl phosphine group, a C₆-C₆₀ arylphosphine oxide group, and a C₆-C₆₀ arylamine group, or a plurality of R₃ are the same as or different from each other;
m is an integer of 1 or 2; and
the alkyl group, alkenyl group, alkynyl group, aryl group, heteroaryl group, aryloxy group, alkyloxy group, cycloalkyl group, heterocycloalkyl group, alkylsilyl group, arylsilyl group, alkylboron group, arylboron group, arylphosphine group, arylphosphine oxide group, and arylamine group of Ar₁ to Ar₂, and R₁ to R₃ may be each independently substituted with at least one substituent selected from the group consisting of a deuterium (D), a halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 ring atoms, a C₆ to Coo aryl group, a heteroaryl group having 5 to 60 ring atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to Coo arylphosphine oxide group, and a C₆ to C₆₀ arylamine group, and wherein when the substituent is present in a plural number, they may be the same or different from each other.

28. The organic electroluminescent device of claim 27, wherein the X₁ to X₃ containing ring is selected from the group of substituents represented by the following structural formulas: 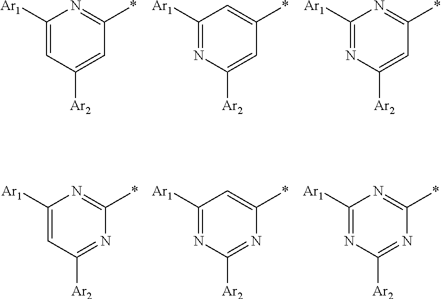
wherein in the formulas,
* indicates a site where a bond with Chemical Formula 1 is made,
Ar₁ and Ar₂ are the same as defined in claim 27.

29. The organic electroluminescent device of claim 27, wherein Ar₁ and Ar₂ are the same as or different from each other, and are each independently selected from the following structural formulas: 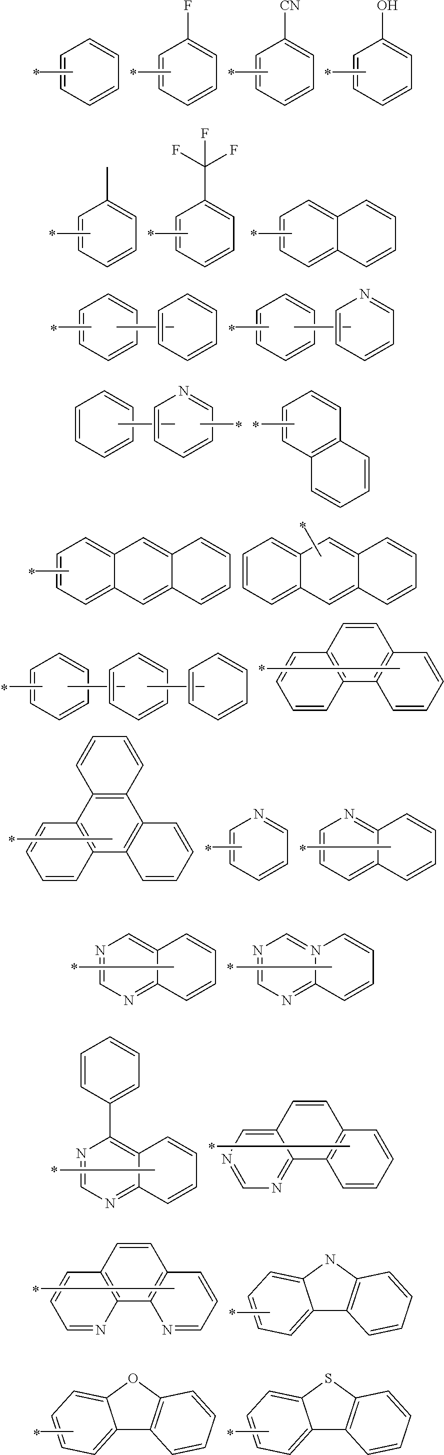 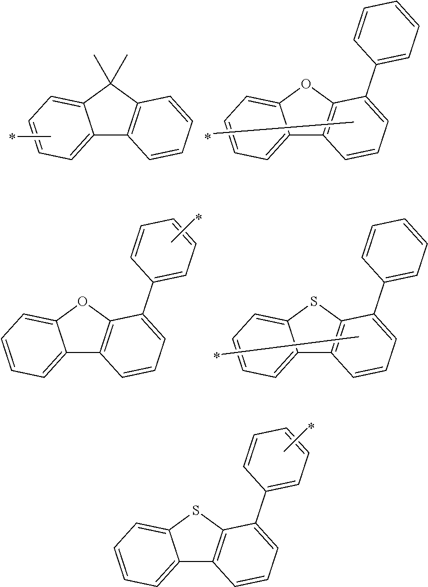
wherein in the formulas,
* indicates a site where a bond with Chemical Formula 1 is made.

30. The organic electroluminescent device of claim 27, wherein the X₄ to X₆ containing ring is selected from the group of substituents represented by the following structural formulas: 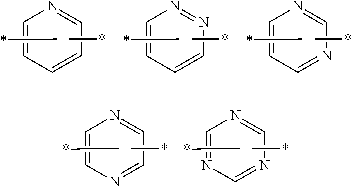
wherein in the formulas,
* indicates a site where a bond with Chemical Formula 1 is made.

31. The organic electroluminescent device of claim 27, wherein the compound represented by Chemical Formula 1 is a compound represented by any one of Chemical Formula 2 to Chemical Formula 3 below: 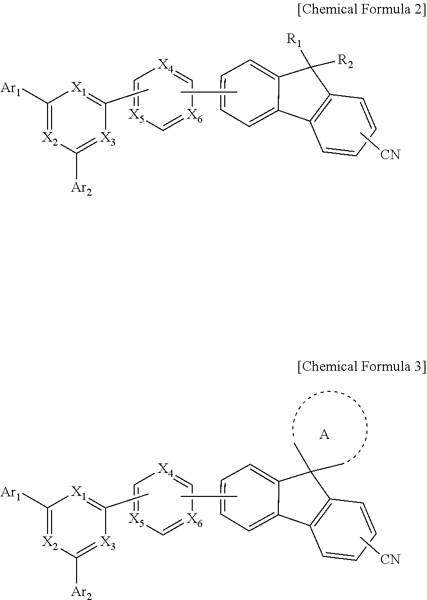
wherein in the formulas,
Ring A is an aliphatic hydrocarbon ring group having 5 to 10 carbon atoms, and
X₁ to X₆, Ar₁ to Ar₂, and R₁ to R₂ are the same as defined in claim 27.

32. The organic electroluminescent device of claim 27, wherein the compound represented by Chemical Formula 1 is a compound represented by any one of Chemical Formula 8 to Chemical Formula 11 below: 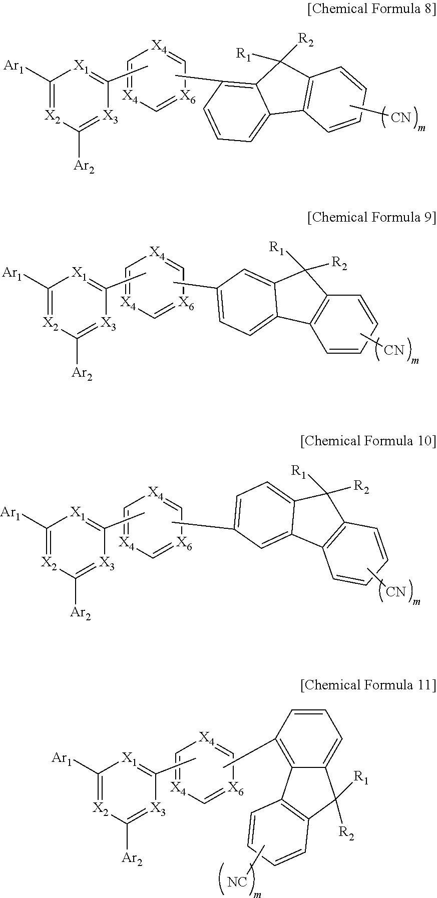
wherein in the formulas 4 to 7,
X₁ to X₆, Ar₁ to Ar₂, R₁ to R₂ and m are the same as defined in claim 27.

33. The organic electroluminescent device of claim 27, wherein the compound represented by Chemical Formula 1 is a compound represented by any one of Chemical Formula 12 to Chemical Formula 14 below: 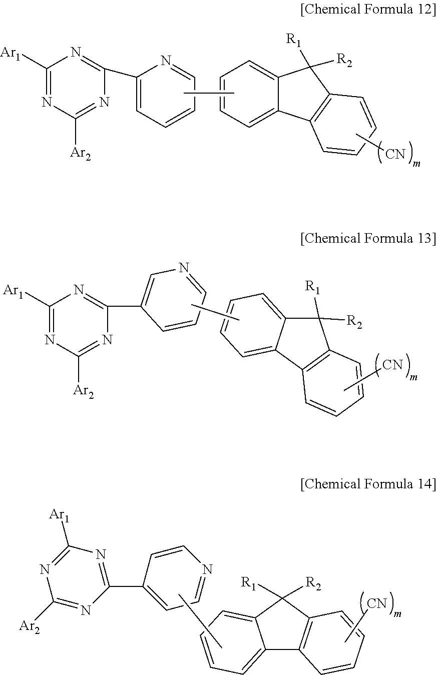
wherein in the formulas 12 to 14,
Ar₁ to Ar₂, R₁ to R₂ and m are the same as defined in claim 27.

34. The organic electroluminescent device of claim 27, wherein the compound is included as at least one of a light emitting layer, a light emitting auxiliary layer, an electron transport layer, and an auxiliary electron transport layer:
